SEQUENCE LISTING (33 sequences)

SEQ ID NO: 1 - DNA, 1971 bp, Homo sapiens
atgacaggagcaaagaggaaaaagaaaagcatgctttggagcaagatgcataccccccagtgtgaagacattatacagtggtgtagaaggcgactgcccattttggattgggcaccacattacaatctgaaagaaaacttgcttccagacactgtgtctgggataatgttggcagttcaacaggtgacccaaggattggcctttgctgttctctcatctgtgcacccagtgtttggtttatatgggtctctgtttcctgccataatttatgccatatttggaatgggacatcatgttgccacaggcacctttgccttgacatccttaatatcagccaacgccgtggaacggattgtccctcagaacatgcagaatctcaccacacagagtaacacaagcgtgctgggcttatccgactttgaaatgcaaaggatccacgttgctgcagcagtttccttcttgggaggtgtgattcaggtggccatgtttgtgctgcaactgggcagtgccacatttgtggtcacagagcctgtgatcagcgcaatgacaactggggctgccacccatgtggtgacttcacaagtcaaatatctcttgggaatgaaaatgccatatatatccggaccacttggattcttttatatttatgcatatgtttttgaaaacatcaagtctgtgcgactggaagcattgcttttatccttgctgagcattgtggtccttgttcttgttaaagagctgaatgaacagtttaaaaggaaaattaaagttgttcttcctgtagatttagttttgattattgctgcatcatttgcttgttattgcaccaatatggaaaacacatatggattagaagtagttggtcatattccacaaggaattccctcacctagagctcccccgatgaacatcctctctgcggtgatcactgaagctttcggagtggcacttgtaggctatgtggcctcactggctcttgctcaaggatctgccaaaaaattcaaatattcaattgatgacaaccaggaatttttggcccatggcctcagcaatatagtttcttcatttttcttctgcataccaagtgctgctgccatgggaaggacggctggcctgtacagcacaggagcgaagacacaggtggcttgtctaatatcttgcattttcgtccttatagtcatctatgcaataggacctttgctttactggctgcccatgtgtgtccttgcaagcattattgttgtgggactgaagggaatgctaatacagttccgagatttaaaaaaatattggaatgtggataaaatcgattggggaatatgggtcagtacatatgtatttacaatatgctttgctgccaatgtgggactgctgtttggtgttgtttgtaccatagctatagtgataggacgcttcccaagagcaatgactgtaagtataaaaaatatgaaagaaatggaatttaaagtgaagacagaaatggacagtgaaaccctgcagcaggtgaaaattatctcaataaacaacccgcttgttttcctgaatgcaaaaaaattttatactgatttaatgaacatgatccaaaaggaaaatgcctgtaatcagccacttgatgatatcagcaagtgtgaacaaaacacattgcttaattccctatccaatggcaactgcaatgaagaagcttcacagtcctgccctaatgagaagtgttatttaatcctggattgcagtggatttaccttttttgactattctggagtctccatgcttgttgaggtttacatggactgtaaaggcaggagtgtggatgtattgttagcccattgtacagcttccttgataaaagcaatgacgtattatggaaacctagactcagagaaaccaattttttttgaatcggtatctgctgcaataagtcatatccattcaaataagaatttgagcaaactcagtgaccacagtgaagtctga

SEQ ID NO: 2 - protein, 656 aa, Homo sapiens
MTGAKRKKRSVLWGKMHTPHREDIKQWCKRRLPILEWAPQYNLKENLLPDTVSGIMLAVQQVAQGLSFAMLSSVHPVFGLYGSLFPAIIYAIFGMGRHVATGTFALTSLISANAVERLVPQSSRNLTTQSNSSVLGLSEFELQRIGVAAAVSFLGGVIQLVMFVLQLGSATFLLTEPVISAMTTGAATHVVTSQVKYLLGIKMPYISGPLGFFYIYAYVFENIKSVQLEALLLSLLSIIVLVLVKELNEQFKRKIKVVLPVDLVLIIAASFACYCTNMENTYGLEVVGHIPNGIPPPRAPPMNILSAVLTEAFGVALVGYVASLALAQGSAKKFKYSVDDNQEFLAHGLSNVIPSFLFCIPSAAAMGRTAGLYSTGAKTQVACLISCIFVLIVIYAIGPLLYWLPMCVLASIIVVGLKGMLIQFRDLKKYWNVDKIDWGIWISTYIFTICFAANVGLLFGVICTIAIVLGRFPRAKTLSITDMKEMELKVKTEMHDETSQQIKIISINNPLVFLNAKKFSADLMKIILKESDSNQPLDDVSKCEQNTLLSSLSNGNCNEEASQPCSSEKCSLVLNCSGLTFFDYTGVSTLVELYLDCKSRSVDVFLANCTASLIKAMTYYGDLDTEKPIFFDSVPAAITIIQSNKNLSKASDHSEV

SEQ ID NO: 3 - DNA, 2289 bp, Mus musculus
tactacaggagggaatctgaaaatctgaaaaaatgacgggagcaaagaggaaaaagagaagcgtgctgtggggcaagatgcacaccccccaccgagaagacattaagcagtggtgtaagaggcgcctacctatcttggaatgggcaccgcagtacaatctgaaggaaaacctgcttccagacactgtgtctgggataatgttggcagttcaacaggtggcacaagggctgtccttcgctatgctttcatctgtacacccagtttttggtttatatggatctctgtttccagccatcatttatgctatatttggaatggggcgccatgttgccacaggcacctttgccttgacatccttgatatcagccaacgctgtggaacgactggtccctcagagcagcaggaacctcaccacacagagcaactcaagtgtgttgggcttatccgagtttgagctgcagagaatcggggttgcagcggctgtttctttcttgggtggagtgattcagctggtcatgtttgtgctgcagctgggcagtgccacattcctgcttacagagcctgtgatcagcgccatgaccacaggggccgccacccatgtcgtgacatcacaagtcaagtatctcttgggaatcaaaatgccatatatatccggaccactgggattcttttatatttatgcgtatgtctttgagaacatcaagtctgtccagctggaagcactgctcttatctttgctgagcatcattgtccttgttctcgttaaagagctgaatgaacagtttaaaagaaaaattaaagttgttcttcctgtcgacttagttttgatcattgccgcctcatttgcttgctattgtaccaacatggaaaacacatacggattagaagtagttgggcacattccaaacgggattcctcctccccgtgctcccccaatgaacatcctctctgcagtgctcactgaagctttcggagttgcacttgtaggctatgtggcctcgctggctctcgcacaaggatctgccaagaagttcaaatattcagttgatgacaaccaggaatttttggcccatggcctcagcaacgtgatcccttcatttctcttctgcatcccaagcgctgccgccatgggaaggacggctggtttgtacagcaccggcgcaaagacgcaggtggcttgtctaatatcttgcattttcgtccttattgtcatctatgcaataggacctctgctgtactggctgcccatgtgcgttcttgcaagtattattgttgtgggcctgaagggaatgttaatacagttccgggatttaaaaaaatactggaatgtggataaaatcgattggggcatatggatcagtacttacatttttacaatatgctttgctgccaatgtgggattgttgtttggtgttatctgcaccatagctatcgtgttaggacgtttcccaagggcaaagactttaagcataacagacatgaaagaaatggaattgaaggtgaagacagaaatgcatgatgaaacctcgcaacagataaaaatcatctcaataaacaatccacttgttttcctgaatgcaaagaaatttagtgctgatttgatgaaaataatcctaaaggaaagtgacagcaaccaaccacttgatgatgtcagcaagtgtgaacagaacaccttgctcagttccctgtccaacggcaactgcaatgaagaggcttcccagccctgctccagcgagaagtgttctttggtcctgaattgcagtggcttgaccttttttgactacacaggagtctccacgcttgttgagctctaccttgattgcaagagcaggagtgtggatgtgttcttagccaactgtactgcatccttgataaaagcaatgacatactatggagacctagacaccgaaaaaccaattttttttgactcggtacctgctgcgataactatcatccaatcaaataagaatttgagcaaggccagtgaccacagtgaagtctgagacccatgtgttgaagtgcacgcctcatctttagcagctgccccaaagaggccagatcctggcattttgcacacattttggtgtatatacagtgtatcactcagtatgaatgatatgacttggcaactgcatagccactggccaagacttgctgaccttctattttttctaattttccttaataaacagattcatttagttatttcatataggcatcaatatgcatttagctttagtttactgaacagtaaacatgcttttatttcttgcaaaaaaaaaaaaaaaa

SEQ ID NO: 4 - protein, 656 aa, Mus musculus; identical to SEQ ID NO: 2

SEQ ID NO: 5 - DNA, 4703 bp, Homo sapiens
atatgagccagcccaggccccgctacgtggtagacagagccgcatactcccttaccctcttcgacgatgagtttgagaagaaggaccggacatacccagtgggagagaaacttcgcaatgccttcagatgttcctcagccaagatcaaagctgtggtgtttgggctgctgcctgtgctctcctggctccccaagtacaagattaaagactacatcattcctgacctgctcggtggactcagcgggggatccatccaggtcccacaaggcatggcatttgctctgctggccaaccttcctgcagtcaatggcctctactcctccttcttccccctcctgacctacttcttcctggggggtgttcaccagatggtgccaggtacctttgccgttatcagcatcctggtgggtaacatctgtctgcagctggccccagagtcgaaattccaggtcttcaacaatgccaccaatgagagctatgtggacacagcagccatggaggctgagaggctgcacgtgtcagctacgctagcctgcctcaccgccatcatccagatgggtctgggcttcatgcagtttggctttgtggccatctacctctccgagtccttcatccggggcttcatgacggccgccggcctgcagatcctgatttcggtgctcaagtacatcttcggactgaccatcccctcctacacaggcccagggtccatcgtctttaccttcattgacatttgcaaaaacctcccccacaccaacatcgcctcgctcatcttcgctctcatcagcggtgccttcctggtgctggtgaaggagctcaatgctcgctacatgcacaagattcgcttccccatccctacagagatgattgtggtggtggtggcaacagctatctccgggggctgtaagatgcccaaaaagtatcacatgcagatcgtgggagaaatccaacgcgggttccccaccccggtgtcgcctgtggtctcacagtggaaggacatgataggcacagccttctccctagccatcgtgagctacgtcatcaacctggctatgggccggaccctggccaacaagcacggctacgacgtggattcgaaccaggagatgatcgctctcggctgcagcaacttctttggctccttctttaaaattcatgtcatttgctgtgcgctttctgtcactctggctgtggatggagctggaggaaaatcccaggtggccagcctgtgtgtgtctctggtggtgatgatcaccatgctggtcctggggatctatctgtatcctctccctaagtctgtgctaggagccctgatcgctgtcaatctcaagaactccctcaagcaactcaccgacccctactacctgtggaggaagagcaagctggactgttgcatctgggtagtgagcttcctctcctccttcttcctcagcctgccctatggtgtggcagtgggtgtcgccttctccgtcctggtcgtggtcttccagactcagtttcgaaatggctatgcactggcccaggtcatggacactgacatttatgtgaatcccaagacctataatagggcccaggatatccaggggattaaaatcatcacgtactgctcccctctctactttgccaactcagagatcttcaggcaaaaggtcatcgccaagacaggcatggacccccagaaagtattactagccaagcaaaaatacctcaagaagcaggagaagcggagaatgaggcccacacaacagaggaggtctctattcatgaaaaccaagactgtctccctgcaggagctgcagcaggactttgagaatgcgccccccaccgaccccaacaacaaccagaccccggctaacggcaccagcgtgtcctatatcaccttcagccctgacagctcctcacctgcccagagtgagccaccagcctccgctgaggcccccggcgagcccagtgacatgctggccagcgtcccacccttcgtcaccttccacaccctcatcctggacatgagtggagtcagcttcgtggacttgatgggcatcaaggccctggccaagctgagctccacctatgggaagatcggcgtgaaggtcttcttggtgaacatccatgcccaggtgtacaatgacattagccatggaggcgtctttgaggatgggagtctagaatgcaagcacgtctttcccagcatacatgacgcagtcctctttgcccaggcaaatgctagagacgtgaccccaggacacaacttccaaggggctccaggggatgctgagctctccttgtacgactcagaggaggacattcgcagctactgggacttagagcaggagatgttcgggagcatgtttcacgcagagaccctgaccgccctgtgagggctcagccagtcctcatgctgcctacagagtgcctggcacttgggacttccataaaggatgagcctggggtcacagggggtgtcgggcggaggaaagtgcatcccccagagcttgggttcctctctcctctccccctctctcctcccttccttccctccccgcatctccagagagagcctctcagcagcaggggggtgctacccttacgggagtgagagtctggtgagcccactcttcacccgtcaggccctggccgcaatggacaagcctcctgctcactccaccccacccacatctgccctgtccttggcagctgaaggacaccttgacttccagcttttacgagtgagccaaaaacagaaggacaagtacaactgtgctggcctgctgtacaagcttcaaaaagtgtcccagagcccgcacggctcggtgtcagatggtgtcaggctgtcacggacatagggataaacttggttaggactctggcttgccttccccagctgcctcaactctgtctctggcagctctgcacccagggaccatgtgctctccacacccaggagtctaggccttggtaactatgcgccccccctccatcatccccaaggctgcccaaaccaccactgctgtcagcaagcacatcagactctagcctggacagtggccaggaccgtcgagaccaccagagctacctccccggggacagcccactaaggttctgcctcagcctcctgaaacatcactgccctcagaggctgctcccttcccctggaggctggctagaaaccccaaagagggggatgggtagctggcagaatcatctggcatcctagtaatagataccagttattctgcacaaaacttttgggaattcctctttgcacccagagactcagaggggaagagggtgctagtaccaacacagggaaaacggatgggacctgggcccagacagtcccccttgaccccagggcccatcagggaaatgcctccctttggtaaatctgccttatccttctttacctggcaaagagccaatcatgttaactcttccttatcagcctgtggcccagagacacaatggggtccttctgtaggcaaaggtggaagtcctccagggatccgctacatcccctaactgcatgcagatgtggaaagaggctgatccagattgggtcttcctgcacaggaagactctttaacacccttaggacctcaggccatcttctcctatgaagatgaaaataggggttaagttttccatatgtacaaggaggtattgagaggaaccctactgttgacttgaaaataaataggttccatgtgtaagtgttttgtaaaatttcagtggaaatgcacagaaaatcttctggcctctcatcactgcttttctcaagcttcttcagcttaacaaccccttccctaacaggttgggctggcccagcctaggaaaacatccccatttctaacttcagccagacctgcgttgtgtgtctgtgtgttgagtgagctggtcagctaacaagtcttcttagagttaaaggagggggtgctggccaagagccaacacattcttggcccaggagcattgcttttctgtgaattcattatgccatctggctgccaatggaactcaaaacttggaaggcgaaggacaatgttatctgggattcaccgtgcacagcacccgaagtgccaaattccaggaggacaagagccttagccaatgacaactcactctcccctactccacctccttccaagtccagctcaggcccaggaggtgggagaaggtcacagagcctcaggaatttccaagtcagagtcccctttgaaccaagtatctagatcccctgaggacttgatgaagtgatccttaacccccaagtaatcattaacccccagaccagcctcagaactgaaggagattgttgacccagtgacctggagttgaggctcagggagagatctgccacatgtctgagggttgcagagcccgctgtggaggtaagattggaaacacatgaggcagagggaagacattgaagaaaacatctctgctggaatatttggaaaagaacactcttctggacctggttgaagcaggaaagatggaggcaaagtagtgaaataatccagaatttcaatgcttttgaatgttcttagtgatactgacctgtgataatataattcccagggaggactgggaaccttatctcttgagatatttgcataatttatttaatttaagcctcattctccttttgttcattttggtaataaactggatttgaattgtgaacaaaaaaaaaaaaaaaaaaa

SEQ ID NO: 6 - protein, 791 aa, Homo sapiens
MSQPRPRYVVDRAAYSLTLFDDEFEKKDRTYPVGEKLRNAFRCSSAKIKAVVFGLLPVLSWLPKYKIKDYIIPDLLGGLSGGSIQVPQGMAFALLANLPAVNGLYSSFFPLLTYFFLGGVHQMVPGTFAVISILVGNICLQLAPESKFQVFNNATNESYVDTAAMEAERLHVSATLACLTAIIQMGLGFMQFGFVAIYLSESFIRGFMTAAGLQILISVLKYIFGLTIPSYTGPGSIVFTFIDICKNLPHTNIASLIFALISGAFLVLVKELNARYMHKIRFPIPTEMIVVVVATAISGGCKMPKKYHMQIVGEIQRGFPTPVSPVVSQWKDMIGTAFSLAIVSYVINLAMGRTLANKHGYDVDSNQEMIALGCSNFFGSFFKIHVICCALSVTLAVDGAGGKSQVASLCVSLVVMITMLVLGIYLYPLPKSVLGALIAVNLKNSLKQLTDPYYLWRKSKLDCCIWVVSFLSSFFLSLPYGVAVGVAFSVLVVVFQTQFRNGYALAQVMDTDIYVNPKTYNRAQDIQGIKIITYCSPLYFANSEIFRQKVIAKTGMDPQKVLLAKQKYLKKQEKRRMRPTQQRRSLFMKTKTVSLQELQQDFENAPPTDPNNNQTPANGTSVSYITFSPDSSSPAQSEPPASAEAPGEPSDMLASVPPFVTFHTLILDMSGVSFVDLMGIKALAKLSSTYGKIGVKVFLVNIHAQVYNDISHGGVFEDGSLECKHVFPSIHDAVLFAQANARDVTPGHNFQGAPGDAELSLYDSEEDIRSYWDLEQEMFGSMFHAETLTAL

SEQ ID NO: 7 - DNA, 4526 bp, Homo sapiens
atatgagccagcccaggccccgctacgtggtagacagagccgcatactcccttaccctcttcgacgatgagtttgagaagaaggaccggacatacccagtgggagagaaacttcgcaatgccttcagatgttcctcagccaagatcaaagctgtggtgtttgggctgctgcctgtgctctcctggctccccaagtacaagattaaagactacatcattcctgacctgctcggtggactcagcgggggatccatccaggtcccacaaggcatggcatttgctctgctggccaaccttcctgcagtcaatggcctctactcctccttcttccccctcctgacctacttcttcctggggggtgttcaccagatggtgccaggtacctttgccgttatcagcatcctggtgggtaacatctgtctgcagctggccccagagtcgaaattccaggtcttcaacaatgccaccaatgagagctatgtggacacagcagccatggaggctgagaggctgcacgtgtcagctacgctagcctgcctcaccgccatcatccagatgggtctgggcttcatgcagtttggctttgtggccatctacctctccgagtccttcatccggggcttcatgacggccgccggcctgcagatcctgatttcggtgctcaagtacatcttcggactgaccatcccctcctacacaggcccagggtccatcgtctttaccttcattgacatttgcaaaaacctcccccacaccaacatcgcctcgctcatcttcgctctcatcagcggtgccttcctggtgctggtgaaggagctcaatgctcgctacatgcacaagattcgcttccccatccctacagagatgattgtggtggtggtggcaacagctatctccgggggctgtaagatgcccaaaaagtatcacatgcagatcgtgggagaaatccaacgcgggttccccaccccggtgtcgcctgtggtctcacagtggaaggacatgataggcacagccttctccctagccatcgtgagctacgtcatcaacctggctatgggccggaccctggccaacaagcacggctacgacgtggattcgaaccaggagatgatcgctctcggctgcagcaacttctttggctccttctttaaaattcatgtcatttgctgtgcgctttctgtcactctggctgtggatggagctggaggaaaatcccaggtggccagcctgtgtgtgtctctggtggtgatgatcaccatgctggtcctggggatctatctgtatcctctccctaagtctgtgctaggagccctgatcgctgtcaatctcaagaactccctcaagcaactcaccgacccctactacctgtggaggaagagcaagctggactgttgcatctgggtagtgagcttcctctcctccttcttcctcagcctgccctatggtgtggcagtgggtgtcgccttctccgtcctggtcgtggtcttccagactcagtttcgaaatggctatgcactggcccaggtcatggacactgacatttatgtgaatcccaagacctataatagggcccaggatatccaggggattaaaatcatcacgtactgctcccctctctactttgccaactcagagatcttcaggcaaaaggtcatcgccaagacaggcatggacccccagaaagtattactagccaagcaaaaatacctcaagaagcaggagaagcggagaatgaggcccacacaacagaggaggtctctattcatgaaaaccaagactgtctccctgcaggagctgcagcaggactttgagaatgcgccccccaccgaccccaacaacaaccagaccccggctaacggcaccagcgtgtcctatatcaccttcagccctgacagctcctcacctgcccagagtgagccaccagcctccgctgaggcccccggcgagcccagtgacatgctggccagcgtcccacccttcgtcaccttccacaccctcatcctggacatgagtggagtcagcttcgtggacttgatgggcatcaaggccctggccaagctgagctccacctatgggaagatcggcgtgaaggtcttcttggtgaacatccatgcccaggtgtacaatgacattagccatggaggcgtctttgaggatgggagtctagaatgcaagcacgtctttcccagcatacatgacgcagtcctctttgcccaggcaaatgctagagacgtgaccccaggacacaacttccaaggggctccaggggatgctgagctctccttgtacgactcagaggaggacattcgcagctactgggacttagagcaggagatgttcgggagcatgtttcacgcagagaccctgaccgccctagagagcctctcagcagcaggggggtgctacccttacgggagtgagagtctggtgagcccactcttcacccgtcaggccctggccgcaatggacaagcctcctgctcactccaccccacccacatctgccctgtccttggcagctgaaggacaccttgacttccagcttttacgagtgagccaaaaacagaaggacaagtacaactgtgctggcctgctgtacaagcttcaaaaagtgtcccagagcccgcacggctcggtgtcagatggtgtcaggctgtcacggacatagggataaacttggttaggactctggcttgccttccccagctgcctcaactctgtctctggcagctctgcacccagggaccatgtgctctccacacccaggagtctaggccttggtaactatgcgccccccctccatcatccccaaggctgcccaaaccaccactgctgtcagcaagcacatcagactctagcctggacagtggccaggaccgtcgagaccaccagagctacctccccggggacagcccactaaggttctgcctcagcctcctgaaacatcactgccctcagaggctgctcccttcccctggaggctggctagaaaccccaaagagggggatgggtagctggcagaatcatctggcatcctagtaatagataccagttattctgcacaaaacttttgggaattcctctttgcacccagagactcagaggggaagagggtgctagtaccaacacagggaaaacggatgggacctgggcccagacagtcccccttgaccccagggcccatcagggaaatgcctccctttggtaaatctgccttatccttctttacctggcaaagagccaatcatgttaactcttccttatcagcctgtggcccagagacacaatggggtccttctgtaggcaaaggtggaagtcctccagggatccgctacatcccctaactgcatgcagatgtggaaagaggctgatccagattgggtcttcctgcacaggaagactctttaacacccttaggacctcaggccatcttctcctatgaagatgaaaataggggttaagttttccatatgtacaaggaggtattgagaggaaccctactgttgacttgaaaataaataggttccatgtgtaagtgttttgtaaaatttcagtggaaatgcacagaaaatcttctggcctctcatcactgcttttctcaagcttcttcagcttaacaaccccttccctaacaggttgggctggcccagcctaggaaaacatccccatttctaacttcagccagacctgcgttgtgtgtctgtgtgttgagtgagctggtcagctaacaagtcttcttagagttaaaggagggggtgctggccaagagccaacacattcttggcccaggagcattgcttttctgtgaattcattatgccatctggctgccaatggaactcaaaacttggaaggcgaaggacaatgttatctgggattcaccgtgcacagcacccgaagtgccaaattccaggaggacaagagccttagccaatgacaactcactctcccctactccacctccttccaagtccagctcaggcccaggaggtgggagaaggtcacagagcctcaggaatttccaagtcagagtcccctttgaaccaagtatctagatcccctgaggacttgatgaagtgatccttaacccccaagtaatcattaacccccagaccagcctcagaactgaaggagattgttgacccagtgacctggagttgaggctcagggagagatctgccacatgtctgagggttgcagagcccgctgtggaggtaagattggaaacacatgaggcagagggaagacattgaagaaaacatctctgctggaatatttggaaaagaacactcttctggacctggttgaagcaggaaagatggaggcaaagtagtgaaataatccagaatttcaatgcttttgaatgttcttagtgatactgacctgtgataatataattcccagggaggactgggaaccttatctcttgagatatttgcataatttatttaatttaagcctcattctccttttgttcattttggtaataaactggatttgaattgtgaacaaaaaaaaaaaaaaaaaaa

SEQ ID NO: 8 - protein, 887 aa, Homo sapiens
MSQPRPRYVVDRAAYSLTLFDDEFEKKDRTYPVGEKLRNAFRCSSAKIKAVVFGLLPVLSWLPKYKIKDYIIPDLLGGLSGGSIQVPQGMAFALLANLPAVNGLYSSFFPLLTYFFLGGVHQMVPGTFAVISILVGNICLQLAPESKFQVFNNATNESYVDTAAMEAERLHVSATLACLTAIIQMGLGFMQFGFVAIYLSESFIRGFMTAAGLQILISVLKYIFGLTIPSYTGPGSIVFTFIDICKNLPHTNIASLIFALISGAFLVLVKELNARYMHKIRFPIPTEMIVVVVATAISGGCKMPKKYHMQIVGEIQRGFPTPVSPVVSQWKDMIGTAFSLAIVSYVINLAMGRTLANKHGYDVDSNQEMIALGCSNFFGSFFKIHVICCALSVTLAVDGAGGKSQVASLCVSLVVMITMLVLGIYLYPLPKSVLGALIAVNLKNSLKQLTDPYYLWRKSKLDCCIWVVSFLSSFFLSLPYGVAVGVAFSVLVVVFQTQFRNGYALAQVMDTDIYVNPKTYNRAQDIQGIKIITYCSPLYFANSEIFRQKVIAKTGMDPQKVLLAKQKYLKKQEKRRMRPTQQRRSLFMKTKTVSLQELQQDFENAPPTDPNNNQTPANGTSVSYITFSPDSSSPAQSEPPASAEAPGEPSDMLASVPPFVTFHTLILDMSGVSFVDLMGIKALAKLSSTYGKIGVKVFLVNIHAQVYNDISHGGVFEDGSLECKHVFPSIHDAVLFAQANARDVTPGHNFQGAPGDAELSLYDSEEDIRSYWDLEQEMFGSMFHAETLTALESLSAAGGCYPYGSESLVSPLFTRQALAAMDKPPAHSTPPTSALSLAAEGHLDFQLLRVSQKQKDKYNCAGLLYKLQKVSQSPHGSVSDGVRLSRT

SEQ ID NO: 9 - DNA, 3049 bp, Mus musculus
caaagcttgtcaatgtcccagacatgaaccagcccaggccccgctacgtggtagacagagctgcgtactccctctccctcttcgatgatgagtttgagaagaaggaccgtgcctacccggtgggagagaagcttcgcaacactttcaggtgctcctcagccaagttcaaagcctttgtgtttgggctgttgcctgtgctctcctggctccctaaatacaagatcaaagactacatcatccctgacttgctgggtggactcagtggagggtgtatccaggtgccacaaggcatggcatttgcattgctggccaaccttcctgctgtcaacggtctctactcctccttcttccccctcctgacctatttcttcttagggggtattcaccagatggtgccaggtacctttgccgttatcagcatcctggtgggtaacatctgtctgcagctggccccagagtcgaaattccagatcttcaacaatgtcaccaatgagacctacgtggacacagcagccatggaggcagaaagactgcatgtgtctgcgacacttgcctgcctgactgctgtcatccagatggccttgggcttcatgcagtttggctttgttgccatctacctctctgagtcctttatccggggcttcatgacagccgctggcctgcagattctcatctctgtgctcaagtacatctttggtctgaccattccctcctatacaggccctggctccatcgtctttaccttcattgacatttgcaaaaacctcccccacaccaacatcgcctcgctcatctttgccctggtcagcggtgtcttcctggtgctggtgaaggaactcaacgctcggtacatgcacaagatccacttccccatccctacagagatgattgtggtggtagttgcaaccgctatctctgggagctgcaagatgccaaaaaaataccacatgcagattgtgggggagatccgacaagggtttcccactccggtggcaccgatggtttcgcagtggaaggacatggtgggcacagccttctccctggcaattgtgggctatgtcatcaatctggctatgggccgcaccctggccagcaagcatggctatgatgtggattctaaccaggagatgattgccctgggctgtagcaacttcttcggctcctttttcaagatccacgtcatttgctgcgctctctcagtcacactggctgtggatggggctggagggaaatcccaggtggccagcttgtgtgtgtctctggtggtgatgatcaccatgctggtcctgggatcctatctgtaccctctccccaaggctgtgctaggtgccctgatcgctgtcaatctcaagaactccctgaagcagctcactgacccctactacctctggaggaagagtaagctagactgttgtgtctgggtggtgagcttcctctcctcgttcttcctgagcctgccctacggtgtcgcagtgggtgtagccttctccatcctggttgtgatcttccagacccagtttcgaaatggctccacactggcccaggtcatggacacggacatctatgtgaaccccaagacctacaacagggcccaggaaattgcgggagttaagattgtcacttactgctcccctctctactttgccaactcagagatcttcaggcaaaaagtcattgccaagacaggtatggacccccagaaagtattactcgccaagcagaaatatctccggaagcaggagaagaggacggcgatacccacacagcagagaaagtccctcttcatgaaaaccaagacggtctctctacaggaactccaacaggactttgaaagtgctccctctactgaccccaacaacaaccaagctcctgcggctgaggctcacatatcctacatcaccttcagtccagacgcctccacagctgctgcatgtgagctgcccgcctccactcggtccccccaggaggctagtgacactctagccagtgtgccgccctttgtcaccttccacactctcatccttgacatgagtggagtcagctttgtggatttgatgggcatcaaagctctcgccaagctaagctccacctatgagaagatcggagtccagatctttctggtaaacatccacgcccaggtgtataatgacatcagccacggaggtgtctttgaagatgggtgtgtacagcgcagtcacgtgttccccagcatccacgatgcagtcctctttgcccaagcaaatgctagagaagccccagaccgcaacttccatggggctccaggggatactgagttctccttgtatgactcagaagaggaaggtcccagctattgggacttagaacaggagatgttcgggaccatgtttcacacagagaccctgactgccctgtgaggacccatttggttcccatatttcctcctgagggcctggcatctggaatttccatggatgatacactcgggattgcaggagtgactgagagaagagtatgtctgccaagatgctagagcctctctcaactctttgttttctcttccacctttccctctctccctgtctccccgggagagagccatctcagcctctgcagctggatgagcaagctctgacaaggatgagactctggtgaacccatctcatcacctcccccttcccactcaagcctgggccaactataagctcatacctcccatcaacgcaagccccaccccatggcagctgacttccagcctttagagtgagctaagaacagaatgataagagcacctgtgctaagctggactgcagttcaaatgtatctgtgtcctgagtgattctgccttggaaggtttcaggctgtggtggaacacagagatgaatggggttacaatggtgaagcttgtccctccggttgctccttctagttgttagctttgtgcctcaaagacactgtactactcacaacccaggagtctctgtctttgtgcccatttccctctcattttcctaagactgcccaggcccccactgctgtccagcacttcagactctagtgtggacagtgg

SEQ ID NO: 10 - protein, 790 aa, Mus musculus
MNQPRPRYVVDRAAYSLSLFDDEFEKKDRAYPVGEKLRNTFRCSSAKFKAFVFGLLPVLSWLPKYKIKDYIIPDLLGGLSGGCIQVPQGMAFALLANLPAVNGLYSSFFPLLTYFFLGGIHQMVPGTFAVISILVGNICLQLAPESKFQIFNNVTNETYVDTAAMEAERLHVSATLACLTAVIQMALGFMQFGFVAIYLSESFIRGFMTAAGLQILISVLKYIFGLTIPSYTGPGSIVFTFIDICKNLPHTNIASLIFALVSGVFLVLVKELNARYMHKIHFPIPTEMIVVVVATAISGSCKMPKKYHMQIVGEIRQGFPTPVAPMVSQWKDMVGTAFSLAIVGYVINLAMGRTLASKHGYDVDSNQEMIALGCSNFFGSFFKIHVICCALSVTLAVDGAGGKSQVASLCVSLVVMITMLVLGSYLYPLPKAVLGALIAVNLKNSLKQLTDPYYLWRKSKLDCCVWVVSFLSSFFLSLPYGVAVGVAFSILVVIFQTQFRNGSTLAQVMDTDIYVNPKTYNRAQEIAGVKIVTYCSPLYFANSEIFRQKVIAKTGMDPQKVLLAKQKYLRKQEKRTAIPTQQRKSLFMKTKTVSLQELQQDFESAPSTDPNNNQAPAAEAHISYITFSPDASTAAACELPASTRSPQEASDTLASVPPFVTFHTLILDMSGVSFVDLMGIKALAKLSSTYEKIGVQIFLVNIHAQVYNDISHGGVFEDGCVQRSHVFPSIHDAVLFAQANAREAPDRNFHGAPGDTEFSLYDSEEEGPSYWDLEQEMFGTMFHTETLTAL

SEQ ID NO: 11 - protein, 8 aa, Mus musculus
GTSRHISV

SEQ ID NO: 12 - protein, 98 aa, Mus musculus
GDVMSGLVIGIILVPQAIAYSLLAGLQPIYSLYTSFFANLIYFLNGTSRHVNVGIFSLLCLMVGQVVDRELQLAGFDPSQDSLGPKNNDSTLNNSATT

SEQ ID NO: 13 - protein, 98 aa, Mus musculus
GDVMSGLIVGILLVPQSIAYSLLAGQEPIYGLYTSFFASIIYFLFGTSRHISVGIFGILCLMIGEVVDRELHKACPDTDATSSSIAVFSSGCVVVNHT

SEQ ID NO: 14 - protein, 91 aa, Mus musculus
SDIVSGISTGLVAVLQGLAFALLVNIPPAYGLYAAFFPVITYFFLGTSRHISVGPFPVLSMMVGVVVTRVVSDPNASSELSSSSTENDSFI

SEQ ID NO: 15 - protein, 97 aa, Mus musculus
SDIISGVSTGLVGTLQGMAYALLAAVPVQFGLYSAFFPILTYFVFGTSRHISVGPFPVVSLMVGSVVLSMAPDDHFLVPSGNGSALNSTTLDTGTRD

SEQ ID NO: 16 - protein, 91 aa, Mus musculus
GDLVSGISTGVLQLPQGLAFAMLAAVPPVFGLYSSFYPVIMYCFFGTSRHISIGPFAVISLMIGGVAVRLVPDDIVIPGGVNATNGTEARD

SEQ ID NO: 17 - protein, 85 aa, Mus musculus
GDLLSGLSVAIMQLPQGLAYALLAGLPPMFGLYSSFYPVFIYFLFGTSRHISVGTFAVMSVMVGSVTESLTADKAFVQGLNATAD

SEQ ID NO: 18 - protein, 93 aa, Mus musculus
PDTVSGIMLAVQQVAQGLSFAMLSSVHPVFGLYGSLFPAIIYAIFGMGRHVATGTFALTSLISANAVERLVPQSSRNLTTQSNSSVLGLSEFE

SEQ ID NO: 19 - protein, 97 aa, Mus musculus
GDLLAGLSVGLAQVPQGLILSLLTRQLIPPLNVTYAAFCSSVIYVIFGSCHQMSIGPFFLVSALMINVLKDRPFNNGHLILGTFVKDDFSVPTFYLS

SEQ ID NO: 20 - protein, 94 aa, Mus musculus
PDLLGGLSGGCIQVPQGMAFALLANLPAVNGLYSSFFPLLTYFFLGGIHQMVPGTFAVISILVGNICLQLAPESKFQIFNNVTNETYVDTAAME

SEQ ID NO: 21 - protein, 78 aa, Mus musculus
LDFIAGLSVGLTVIPQALAYAEVAGLPPQYGLYSAFMGCFVYFFLGTSRDVTLGPTAIMSLLVSFYTFREPAYAVLLA

SEQ ID NO: 22 - protein, 23 aa, Homo sapiens
GAAAAATGACAGGAGCAAAGAGG

SEQ ID NO: 23 - DNA, 23 bp, Homo sapiens
acatagcctacaagtgccactcc

SEQ ID NO: 24 - DNA, 32 bp, Homo sapiens
tagacagagccgcatactcccttaccctcttc

SEQ ID NO: 25 - DNA, 31 bp, Homo sapiens
gatgtgcttgctgacagcagtggtggtttgg

SEQ ID NO: 26 - DNA, 33 bp, Mus musculus
caaagcttgtcaatgtcccagacatgaaccagc

SEQ ID NO: 27 - DNA, 36 bp, Mus musculus
ccactgtccacactagagtctgaagtgctggacagc

SEQ ID NO: 28 - DNA, 22 bp, Homo sapiens
tgaggacttgatgaagtgatcc

SEQ ID NO: 29 - DNA, 22 bp, Homo sapiens
atgaacaaaaggagaatgaggc

SEQ ID NO: 30 - DNA, 21 bp, Homo sapiens
aaagatggaggcaaagtagtg

SEQ ID NO: 31 - protein, 18 aa, Mus musculus
KRSVLWGKMHTPHREDIK

SEQ ID NO: 32 - protein, 17 aa, Mus musculus
QELQQDFESAPSTDPNN

SEQ ID NO: 33 - protein, 20 aa, Mus musculus
KQKYLRKQEKRTAIPTQQRK

(19) United States
(12) Patent Application Publication
(10) Pub. No.: US 2005/0176945 A1
(43) Pub. Date: Aug. 11, 2005

(54) Cloning and characterization of slc26a7 and slc26a9 anion exchangers

(76) Inventors: David B Mount, Jr., Boston, MA (US); Michael F. Romero, Cleveland, OH (US)

Correspondence Address: JENKINS, WILSON & TAYLOR, P. A., 3100 TOWER BLVD, SUITE 1400, DURHAM, NC, 27707, US

(21) Appl. No.: 10/505,316

(22) PCT Filed: Feb. 28, 2003
(86) PCT No.: PCT/US03/06220

Related U.S. Application Data
(60) Provisional application No. 60/360,287, filed on Feb. 28, 2002.

(51) Int. Cl. C12Q 1/68; C07H 21/04; C07K 14/705; C07K 16/28

(52) U.S. Cl. 536/23.5; 435/6; 435/69.1; 435/320.1; 435/325; 530/350; 530/388.22

(57) ABSTRACT

Isolated nucleic acids encoding SLC26A7 and SLC26A9 anion transporter polypeptides, recombinantly expressed SLC26A7 and SLC26A9 anion transporter polypeptides, heterologous expression systems for recombinant expression of SLC26A7 and SLC26A9 anion transporter polypeptides, assay methods employing the same, and methods for modulation of anion transport activity.

### CROSS REFERENCE TO RELATED APPLICATIONS

This application is based on and claims priority to U.S. Provisional Application Ser. No. 60/360,287, filed Feb. 28, 2002, and entitled CLONING AND CHARACTERIZATION OF SLC26A7 and SLC26A9 ANION EXCHANGERS, herein incorporated by reference in its entirety.

### GRANT STATEMENT

This work was supported by grants RO1 DK57708, PO1 DK038226, RO1 DK56218, and T32 DK07569-12 from the U.S. National Institute of Health. Thus, the U.S. government has certain rights in the invention.

### FIELD OF THE INVENTION

The present invention generally relates to anion transporter polypeptides and anion transport activity mediated by the same. More particularly, the present invention provides isolated nucleic acids encoding SLC26 anion transporter polypeptides, isolated and functional SLC26 anion transporter polypeptides, a heterologous expression system for recombinant expression of SLC26 anion transporter polypeptides, methods for identifying modulators of an anion transporter, and uses thereof.

Table of Abbreviations

| | |
|---|---|
| AE | anion exchanger |
| ATCC | American Type Culture Collection |
| BAC | bacterial artificial chromosome |
| BLAST | basic alignment and search tool |
| CF | cystic fibrosis |
| CFTR | cystic fibrosis transmembrane |
| | conductance regulator |
| cM | centimorgan |
| CMV | cytomegalovirus |
| cRNA | complementary RNA |
| CpG | unmethylated cytosine-guanine |
| | dinucleotides |
| DIDS | 4,4′-diisothiocyanatostilbene-2,2′-disulfonic |
| | acid |
| DTST | diastrophic dysplasia sulphate transporter; |
| | SLC26A2 |
| EGFP | enhanced green fluorescent protein |
| EST | expressed sequence tag |
| Fab | antigen-binding antibody fragment |
| FCS | Fluorescence Correlation Spectroscopy |
| Fv | antigen-binding antibody fragment |
| GAPDH | glyceraldehyde-3-phosphate |
| | dehydrogenase |
| GFP | green fluorescent protein |
| HEPES | 4-(2-hydroxyethyl)-1- |
| | piperazineethanesulfonic acid |
| HLA | human leukocyte antigen |
| HTGS | high throughput genomic sequences |
| HUGO | Human Genome Organization |
| I.M.A.G.E. | Integrated Molecular Analysis of |
| | Genomes and their Expression |
| | database |
| LA-PCR | long and accurate PCR |
| LDB | location database |
| MGD | Mouse Genome Database |
| MHC | major histocompatibility complex |
| NMDG | N-methyl-D-glucamine |
| ORF | open reading frame |
| OSM | osmolality |
| PAC | P-1 derived artificial chromosome |
| pCMV-SLC26 | construct encoding SLC26 under the |
| | control of a CMV promoter |
| PCR | polymerase chain reaction |
| PFU | plaque-forming unit |
| pHᵢ | intracellular pH |
| PKA | phosphokinase A |
| PKC | phosphokinase C |
| RACE | rapid amplification of cDNA ends |
| RH | radiation hybrid |
| RT-PCR | reverse transcription - polymerase chain |
| | reaction |
| Sat-1 | sulphate anion transporter-1 (SLC26A1) |
| SDS | sodium dodecyl sulphate |
| SELDI-TOF | Surface-Enhanced Laser |
| | Desorption/Ionization Time-Of-flight |
| | Spectroscopy |
| SLC26 | solute carrier 26 protein family |
| Sp1 | pregnancy-“specific” beta 1-glycoprotein; |
| | Cys₂-His₂ zinc finger transcription |
| | factor |
| SPR | surface plasmon resonance |
| STAS | sulfate transporter and anti-sigma domain |
| STS | sequence-tagged site |
| TESS | Transcription Element Search Software |
| UTR | untranslated region |
| Vₘ | membrane voltage |

### BACKGROUND OF THE INVENTION

Anion exchange at the plasma membrane is primarily mediated by the products of two structurally distinct gene families: (1) the AE (anion exchanger) genes, which form a subset of the bicarbonate transporter SLC4 superfamily (Romero et al., 2000; Tsuganezawa et al., 2001); and (2) the SLC26 or sulphate permease gene family (Everett & Green, 1999). Members of the SLC26 gene family have been identified by expression cloning (Bissig et al., 1994), subtractive cDNA cloning (Zheng et al., 2000), and positional cloning of human disease genes (Everett & Green, 1999).

The SLC26 gene family has been highly conserved during evolution, and homologues have been identified in bacteria, yeast, plants, and animals. See Everett & Green (1999) Hum Mol Genet 8:1883-1891 and Kere et al. (1999) Am J Physiol 276:G7-G13. Four mammalian SLC26 genes have been described (SLC26A1, SLC26A2, SLC26A3, and SLC26A4). The Drosophila genome contains at least nine family members, suggesting that additional mammalian paralogues also exist.

Physiological roles for individual family members include transepithelial salt transport (Everett & Green, 1999; Scott & Karniski, 2000a), thryoidal iodide transport (Scott et al., 1999), development and function of the inner ear (Everett & Green, 1999; Zheng et al., 2000), sulphation of extracellular matrix (Satoh et al., 1998), and renal excretion of bicarbonate (Royaux et al., 2001) and oxalate (Karniski et al., 1998). The various substrates transported by the SLC26 anion exchangers include sulphate (SO₄²⁻), chloride (Cl⁻), iodide (I⁻), formate, oxalate, hydroxyl ion (OH⁻), and bicarbonate (HCO₃⁻) (Bissig et al., 1994; Karniski et al., 1998; Satoh et al., 1998; Moseley et al., 1999a; Scott & Karniski, 2000a; Soleimani et al., 2001).

The multiple physiological roles of SLC26 transporters are supported by diverse anion transport properties. Despite a capacity for versatile anion exchange, SLC26 anion transporters display distinct patterns of anion specificity and cis-inhibition. For example, SLC26A4, also known as pendrin, can transport chloride, hydroxyl ion, bicarbonate, iodide, and formate, but neither oxalate nor sulphate (Scott et al., 1999; Scott & Karniski, 2000a; Royaux et al., 2001; Soleimani et al., 2001).

Thus, there exists a long-felt need in the art to identify and functionally characterize SLC26 anion transporters as pharmaceutical targets for diseases and disorders related to abnormal anion transport activity.

To meet this need, the present invention provides novel SLC26A7 and SLC26A9 anion transporter polypeptides. The present invention also provides methods for identifying and using modulators of anion transport via SLC26A7 and SLC26A9.

### SUMMARY OF INVENTION

The present invention provides isolated SLC26A7 and SLC26A9 polypeptides, and SLC26A7 and SLC26A9 nucleic acids. The polypeptides and nucleic acids are useful in the detection methods and assays disclosed herein. The present invention further provides antibodies that specifically recognize a SLC26A7 polypeptide or a SLC26A9 polypeptide.

A SLC26A7 polypeptide can comprise: (a) a polypeptide of SEQ ID NO:2 or 4; (b) a polypeptide substantially identical to SEQ ID NO:2 or 4; (c) a polypeptide encoded by a nucleic acid molecule of SEQ ID NO:1 or 3; or (d) a polypeptide encoded by a nucleic acid molecule substantially identical to SEQ ID NO:1 or 3.

A SLC26A7 polypeptide can also comprise a polypeptide encoded by an isolated nucleic acid molecule selected from the group consisting of: (a) an isolated nucleic acid molecule encoding a polypeptide of SEQ ID NO:2 or 4; (b) an isolated nucleic acid molecule of SEQ ID NO:1 or 3; (c) an isolated nucleic acid molecule which hybridizes to a nucleic acid of SEQ ID NO:1 or 3 under wash stringency conditions represented by a wash solution having less than about 200 mM salt concentration and a wash temperature of greater than about 45° C., and which encodes a SLC26A7 polypeptide; and (d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A7 polypeptide encoded by the isolated nucleic acid of one of (a), (b), and (c) above.

In a preferred embodiment of the invention, a SLC26A7 polypeptide comprises a functional SLC26A7 polypeptide. The functional property is preferably chloride transport.

A SLC26A7 nucleic acid molecule of the invention preferably comprises a nucleic acid molecule encoding a SLC26A7 polypeptide. A SLC26A7 nucleic acid molecule can comprise: (a) a nucleotide sequence of SEQ ID NO:1 or 3; or (b) a nucleotide sequence substantially identical to SEQ ID NO:1 or 3. A SLC26A7 nucleic acid can also comprise a nucleic acid selected from the group consisting of: (a) an isolated nucleic acid molecule encoding a polypeptide of SEQ ID NO:2 or 4; (b) an isolated nucleic acid molecule of SEQ ID NO:1 or 3; (c) an isolated nucleic acid molecule which hybridizes to a nucleic acid sequence of SEQ ID NO:1 or 3 under wash stringency conditions represented by a wash solution having less than about 200 mM salt concentration and a wash temperature of greater than about 45° C., and which encodes a SLC26A7 polypeptide; and
(d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A7 polypeptide encoded by the isolated nucleic acid of one of (a), (b), and (c) above.

A SLC26A9 polypeptide can comprise: (a) a polypeptide of SEQ ID NO:6, 8, or 10; (b) a polypeptide substantially identical to SEQ ID NO:6, 8, or 10; (c) a polypeptide encoded by a nucleic acid molecule of SEQ ID NO:5, 7, or 9; or (d) a polypeptide encoded by a nucleic acid molecule substantially identical to SEQ ID NO:5, 7, or 9.

A SLC26A9 polypeptide can also comprise a polypeptide encoded by an isolated nucleic acid molecule selected from the group consisting of: (a) an isolated nucleic acid molecule encoding a polypeptide of SEQ ID NO:6, 8, or 10; (b) an isolated nucleic acid molecule of SEQ ID NO:5, 7, or 9; (c) an isolated nucleic acid molecule which hybridizes to a nucleic acid of SEQ ID NO:5, 7, or 9 under wash stringency conditions represented by a wash solution having less than about 200 mM salt concentration and a wash temperature of greater than about 45° C., and which encodes a SLC26A9 polypeptide; and (d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A9 polypeptide encoded by the isolated nucleic acid of one of (a), (b), and (c) above.

In a preferred embodiment of the invention, a SLC26A9 polypeptide comprises a functional SLC26A9 polypeptide. The functional property is preferably chloride transport, bicarbonate ion transport, or a combination thereof.

A SLC26A9 nucleic acid molecule of the invention preferably comprises a nucleic acid molecule encoding a SLC26A9 polypeptide. A SLC26A9 nucleic acid molecule can comprise: (a) a nucleotide sequence of SEQ ID NO:5, 7, or 9; or (b) a nucleotide sequence substantially identical to SEQ ID NO:5, 7, or 9. A SLC26A9 nucleic acid can also comprise a nucleic acid selected from the group consisting of: (a) an isolated nucleic acid molecule encoding a polypeptide of SEQ ID NO:6, 8, or 10; (b) an isolated nucleic acid molecule of SEQ ID NO:5, 7, or 9; (c) an isolated nucleic acid molecule which hybridizes to a nucleic acid sequence of SEQ ID NO:5, 7, or 9 under wash stringency conditions represented by a wash solution having less than about 200 mM salt concentration and a wash temperature of greater than about 45° C., and which encodes a SLC26A9 polypeptide; and (d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A9 polypeptide encoded by the isolated nucleic acid of one of (a), (b), and (c) above.

The present invention further provides methods for detecting a SLC26A7 or SLC26A9 nucleic acid, the method comprising: (a) procuring a biological sample having nucleic acid material; (b) hybridizing the nucleic acid molecule of any one of odd-numbered SEQ ID NOs:1-9 under stringent hybridization conditions to the biological sample of (a), thereby forming a duplex structure between the nucleic acid of any one of odd-numbered SEQ ID NOs:1-9 and a nucleic acid within the biological sample; and (c) detecting the duplex structure of (b), whereby a SLC26A7 or SLC26A9 nucleic acid molecule is detected.

The present invention further provides antibodies that specifically recognize a SLC26A7 or SLC26A9 polypeptide, and methods for producing the same. A representative embodiment of the method comprises: (a) recombinantly or synthetically producing a SLC26A7 polypeptide; (b) formulating the polypeptide of (a) whereby it is an effective immunogen; (c) administering to an animal the formulation of (b) to generate an immune response in the animal comprising production of antibodies, wherein antibodies are present in the blood serum of the animal; and (d) collecting the blood serum from the animal of (c) comprising antibodies that specifically recognize a SLC26A7 polypeptide.

Also provided is a method for detecting a level of a SLC26A7 or SLC26A9 polypeptide. In a representative embodiment, the method comprises (a) obtaining a biological sample having peptidic material; (b) detecting a SLC26A7 polypeptide in the biological sample of (a) by immunochemical reaction with the antibody of the present invention, whereby an amount of SLC26A7 or SLC26A9 polypeptide in a sample is determined.

Also provided are systems for recombinant expression of a SLC26 polypeptide. A recombinant expression system can comprise: (a) a SLC26A7 or SLC26A9 polypeptide of the invention (e.g., representative embodiments set forth as SEQ ID NOs:2, 4, 6, and 8); and (b) a host cell expressing the SLC26A7 or SLC26A9 polypeptide. A host cell can comprise any suitable cell. A preferred host cell comprises a mammalian cell, more preferably a human cell.

Using the disclosed system for recombinant expression of a SLC26A7 or SLC26A9 polypeptide, the present invention further provides a method for identifying modulators of anion transport. Also provided are modulators of anion transport that are identified by the disclosed methods.

In a preferred embodiment of the invention a method for identifying a modulator of anion transport comprises: (a) providing a recombinant expression system whereby a SLC26A7 or SLC26A9 polypeptide is expressed in a host cell, (b) providing a test substance to the system of (a); (c) assaying a level or quality of SLC26A7 or SLC26A9 function in the presence of the test substance; (d) comparing the level or quality of SLC26A7 or SLC26A9 function in the presence of the test substance with a control level or quality of SLC26A7 or SLC26A9 function; and (e) identifying a test substance as an anion transport modulator by determining a level or quality of SLC26A7 or SLC26A9 function in the presence of the test substance as significantly changed when compared to a control level or quality of SLC26A7 or SLC26A9 function.

In another embodiment of the invention, a method for identifying a modulator of anion transport comprises: (a) exposing a SLC26A7 or SLC26A9 polypeptide to one or more test substances; (b) assaying binding of a test substance to the isolated SLC26A7 or SLC26A9 polypeptide; and (c) selecting a candidate substance that demonstrates specific binding to the SLC26A7 or SLC26A9 polypeptide.

The present invention further provides methods for modulating anion transport activity in a subject. Preferably, the subject is a mammalian subject, and more preferably a human subject. Also preferably, the anion transport activity that is altered in a subject comprises an activity of a SLC26A7 or SLC26A9 polypeptide.

In one embodiment of the present invention, a method for modulating anion transport activity in a subject comprises: (a) preparing a composition comprising a SLC26A7 or SLC26A9 modulator identified according to the disclosed methods, and a pharmaceutically acceptable carrier; (b) administering an effective dose of the pharmaceutical composition to a subject, whereby anion transport activity in the subject is altered.

Accordingly, it is an object of the present invention to provide novel SLC26 nucleic acids and polypeptides, methods for detecting a SLC26 nucleic acid, heterologous expression systems whereby a SLC26 polypeptide is expressed, methods and assays employing a heterologous SLC26 expression system, and methods for modulating and detecting a SLC26 polypeptide. This object is achieved in whole or in part by the present invention.

An object of the invention having been stated above, other objects and advantages of the present invention will become apparent to those skilled in the art after a study of the following description of the invention, Figures, and non-limiting Examples.

### BRIEF DESCRIPTION OF THE DRAWINGS

FIG. 1 is an alignment of a conserved SLC26 domain encompassing the Prosite “sulfate transport” signature sequence (Bucher & Bairoch, 1994; Hofmann et al., 1999) (http://www.expasy.ch/prosite/) in mouse SLC26A1 (SEQ ID NO:12), mouse SLC26A2 (SEQ ID NO:13), mouse SLC26A3 (SEQ ID NO:14), mouse SLC26A4 (SEQ ID NO:15), mouse SLC26A5 (SEQ ID NO:16), mouse SLC26A6 (SEQ ID NO:17), mouse SLC26A7 (SEQ ID NO:18), mouse SLC26A8 (SEQ ID NO:19), mouse SLC26A9 (SEQ ID NO:20), and mouse SLC26A1 (SEQ ID NO:21). The 22-residue Prosite motif is underlined in sequences that conform to the consensus (SLC26A1, SLC26A2, SLC26A3, and SLC26A11). Shading, similar residues, conservative substitutions, and weakly similar residues; asterisks (*), invariant residues.

FIGS. 2A-2C depict chloride and sulphate transport activity of SLC26A7 and SLC26A9.

FIG. 2A is a bar graph that depicts ³⁵SO₄²⁻ uptake (pmol/oocyte/hr) in oocytes expressing SLC26A2, SLC26A7, or SLC26A9. Control cells (H₂O) were injected with water instead of SLC26 cRNA. Open bars, extracellular pH 7.4; Solid bars, extracellular pH 6.0; hr, hour.

FIG. 2B is a bar graph depicting the effect of extracellular Cl⁻ on ³⁵SO₄²⁻ uptake (pmol/oocyte/hr). Oocytes expressing SLC26A2, SLC26A7, or SLC26A9, or control oocytes (H₂O) were incubated in medium containing ³⁵SO₄²⁻ for one hour. (−), Cl⁻-free medium; (+), 25 mM Cl⁻ added to the medium; hr, hour.

FIG. 2C is a bar graph that presents ³⁶Cl⁻ uptake (pmol/oocyte/h) in oocytes expressing SLC26A2, SLC26A7, or SLC26A9. Control cells (H₂O) were injected with water instead of SLC26 cRNA. Open bars, extracellular pH 7.4; solid bars, extracellular pH 6.0; h, hour; asterisk (*), a statistically significant difference (p<0.05) when compared to water-injected control oocytes.

FIGS. 3A-3C present a functional characterization of chloride uptake via SLC26A7 and SLC26A9.

FIG. 3A is a bar graph that depicts ³⁶Cl⁻ uptake (pmol/oocyte/h) as a function of extracellular osmolality and pH in SLC26A7-injected oocytes (A7) and water injected control oocytes (H₂O). Oocytes were incubated during the pre-uptake period and uptake period at varying osmolaltiy at either pH 7.4 (open bars), pH 6.0 (black bars), or at pH 6.0 in the presence of 1 mM DIDS (grey bars). ISO, isotonic osmolality, 210 mOsm/kg; HYPO, hypotonic osmolality, 120 mOsm/kg; HYPER, hypertonic osmolality, 300 mOsm/kg; h, hour.

FIG. 3B is a bar graph that depicts DIDS inhibition of ³⁶Cl⁻ uptake (pmol/oocyte/h) in SLC26A9injected oocytes (Slc26a9) and water-injected control oocytes (H₂O). Oocytes were incubated during the pre-uptake period and uptake period at either pH 7.4 (open bars), pH 6.0 (black bars), or at pH 6.0 in the presence of 1 mM DIDS (grey bars). H, hour.

FIG. 3C is a bar graph that depicts a lack of cis-inhibition of Cl⁻-Cl⁻ exchange in oocytes expressing SLC26A9 or in control oocytes (H₂O). Oocytes were incubated in medium containing ³⁶Cl⁻ for one hour in the absence (control) or presence of 25 mM of the indicated anions.

FIGS. 4A-4B depict oxalate and formate transport mediated by SLC26A6, SLC26A7, and SLC26A9.

FIG. 4A is a bar graph showing oxalate uptake (pmol/oocyte/hr) in oocytes expressing SLC26A6 (A6), SLC26A7 (A7), or SLC26A9 (A9). Control oocytes (H₂O) were injected with water instead of SLC26 cRNA. Hr, hour.

FIG. 4B is a bar graph showing formate uptake (pmol/oocyte/hr) in oocytes expressing SLC26A6 (A6), SLC26A7 (A7), or SLC26A9 (A9). Control oocytes (H₂O) were injected with water instead of SLC26 cRNA. Hr, hour.

FIGS. 5A-5B present a functional characterization of SLC26A9 using ion-selective microelectrodes as described in Example 8.

FIG. 6 depicts Western blotting of oocyte lysates containing the indicated SLC26 proteins, using a 1:300 titre of a C-terminal Slc26a9-specific antibody; only the core 90 kDa and glycosylated 120 kDa Slc26a9 proteins are detected by the antibody.

FIG. 7 depicts Western blotting of oocyte lysates containing the indicated SLC26 proteins, using a 1:300 titre of an N-terminal Slc26a7-specific antibody; the core 70 kDa and glycosylated 90 kDa Slc26a7 proteins are detected by the antibody only in the Slc26a7 lane.

### BRIEF DESCRIPTION OF SEQUENCES IN THE SEQUENCE LISTING

Odd-numbered SEQ ID NOs:1-9 are nucleotide sequences described in Table 1. Even-numbered SEQ ID NOs:2-10 are protein sequences encoded by the immediately preceding nucleotide sequence, e.g., SEQ ID NO:2 is the protein encoded by the nucleotide sequence of SEQ ID NO:1, SEQ ID NO:4 is the protein encoded by the nucleotide sequence of SEQ ID NO:3, etc.

SEQ ID NO:11 is a SLC26 conserved domain.

SEQ ID NOs:12-21 are the SLC26 sequences indicated in Table 1, each sequence encompassing the Prosite “sulphate transport” signature sequence (Bucher & Bairoch, 1994; Hofmann et al., 1999) (http://www.expasy.ch/prosite/).

SEQ ID NOs:22-30 are primers.

SEQ ID NO:31 is an N-terminal Slc26a7 peptide that corresponds to residues 8-25 of the predicted protein.

SEQ ID NOs:32 and 33 are C-terminal Slc26a9 peptides that correspond to residues 596-612 and 565-584 of the protein, respectively.

TABLE 1
Sequence Listing Summary

| SEQ ID NO. | Description |
|---|---|
| 1-2 | human SLC26A7 |
| 3-4 | mouse SLC26A7 |
| 5-6 | human SLC26A9a |
| 7-8 | human SLC26A9b |
| 9-10 | mouse SLC26A9 |
| 11 | SLC26 conserved domain |
| 12 | mouse SLC26A1 sulphate transport motif |
| 13 | mouse SLC26A2 sulphate transport motif |
| 14 | mouse SLC26A3 sulphate transport motif |
| 15 | mouse SLC26A4 sulphate transport motif |
| 16 | mouse SLC26A5 sulphate transport motif |
| 17 | mouse SLC26A6 sulphate transport motif |
| 18 | mouse SLC26A7 sulphate transport motif |
| 19 | mouse SLC26A8 sulphate transport motif |
| 20 | mouse SLC26A9 sulphate transport motif |
| 21 | mouse SLC26A11 sulphate transport motif |
| 22-30 | primers |
| 31 | N-terminal Slc26a7 peptide |
| 32-33 | C-terminal Slc26a9 peptides |

### DETAILED DESCRIPTION OF THE INVENTION

I. Definitions

While the following terms are believed to be well understood by one of ordinary skill in the art, the following definitions are set forth to facilitate explanation of the invention.

The terms “a,” “an,” and “the” are used in accordance with long-standing convention to refer to one or more.

The term “about”, as used herein when referring to a measurable value such as a percentage of sequence identity (e.g., when comparing nucleotide and amino acid sequences as described herein below), a nucleotide or protein length, an uptake amount, a pH value, etc. is meant to encompass variations of ±20% or ±10%, more preferably ±5%, even more preferably ±1%, and still more preferably ±0.1% from the specified amount, as such variations are appropriate to perform a disclosed method or otherwise carry out the present invention.

II. SLC26 Nucleic Acids and Polypeptides

The present invention provides novel SLC26 nucleic acids and novel SLC26 polypeptides, including functional SLC26 polypeptides. The term “SLC26A” and terms including “SLC26” (e.g., SLC26A7 and SLC26A9) refer generally to isolated SLC26 nucleic acids, isolated polypeptides encoded by SLC26 nucleic acids, and activities thereof. SLC26 nucleic acids and polypeptides can be derived from any organism.

The term “isolated”, as used in the context of a nucleic acid or polypeptide, indicates that the nucleic acid or polypeptide exists apart from its native environment and is not a product of nature. An isolated nucleic acid or polypeptide can exist in a purified form or can exist in a non-native environment such as a transgenic host cell.

The terms “SLC26” and terms including “SLC26” also refer to polypeptides comprising Na⁺-independent anion transporters that transport SO₄²⁻, Cl⁻, formate, and/or oxalate, and to nucleic acids encoding the same.

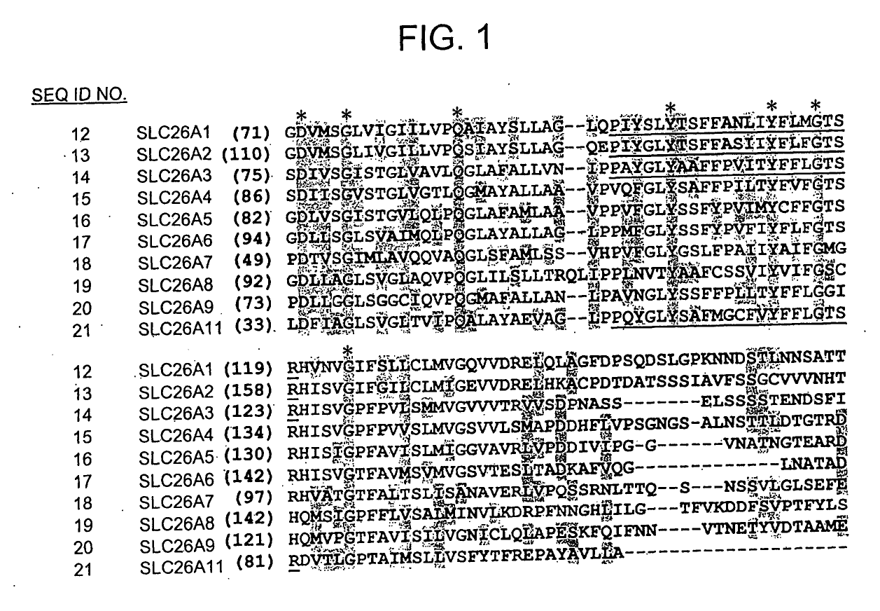

A region within the central hydrophobic core of SLC26 polypeptides includes a 22-residue “sulphate transport” consensus signature, Prosite motif PS01130 (Bucher & Bairoch, 1994; Hofmann et al., 1999) (http://www.expasv.ch/prosite/), which was initially defined by comparison of the first mammalian family members with homologues in lower organisms. An alignment of this region is presented in FIG. 1. SLC26A6 functions as a sulphate transporter, despite its lack of a consensus “sulphate transport” sequence, and thus the functional significance of this sequence motif is unclear. Within this region, many of the SLC26 proteins also share the sequence -GTSRHISV- (SEQ ID NO:11), whereas mouse SLC26A7 and mouse SLC26A9 depart from this consensus. The Prosite sulphate transport region also contains a total of seven invariant residues, which likely play a role in anion transport (FIG. 1).

There is a second cluster of invariant residues at the C-terminal end of the hydrophobic core, in a conserved area defined by Saier et al (Saier et al., 1999). This region includes the triplet -NQE-, residues 417-419 of mouse SLC26A6, which is conservatively variable only in SLC26A7 (-NQD-). Three invariant residues in this section, E419, N425, and L483 in mouse SLC26A2, have been shown to have functional significance in SHST1, a SLC26 homologue from the plant S. hamata (Khurana et al., 2000). Moreover, two of these invariant residues are mutated (N425D and L483P) in patients with a severe defect in human SLC26A2, causing achondrogenesis type 1B and/or atelosteogenesis type 2. The SLC26A2 N425D mutant has further been shown to be non-functional in Xenopus oocytes (Karniski, 1989).

The C-terminal cytoplasmic domain of SLC26 proteins encompasses the STAS LSulphate Transporter and Anti-Sigma) domain, recently defined by the homology between the SLC26 proteins and bacterial anti-sigma factor antagonists (Aravind & Koonin, 2000). Structural features of this domain have been predicted from the NMR analysis of the anti-sigma factor SPOIIAA (Aravind & Koonin, 2000), and include a characteristic α-helical handle. There is also a highly conserved loop interspersed between a α-pleated sheet and α-helix, just upstream of the α-helical handle. This loop and α-pleated sheet have been proposed to play a role in nucleotide binding and hydrolysis, in analogy to the known biochemistry of the anti-sigma factor antagonists (Aravind & Koonin, 2000). The loop is highly conserved in SLC26 proteins and contains two invariant residues, D660 and L667 of mouse SLC26A2.

The STAS domain also contains a highly variable loop just proximal to the β-pleated sheet and putative nucleotide binding loop (Aravind & Koonin, 2000). This variable loop is the site of significant insertions in SLC26 proteins. The largest known insertion comprises 150 amino acids in the case of human SLC26A7. Interestingly, no such insertion is present in bacterial SLC26 homologues, and this loop is the shortest in SLC26A9, which is arguably the most primeval of the mammalian SLC26 paralogs.

The present invention provides novel SLC26A7 and SLC26A9 polypeptides, SLC26A7 and SLC26A9 nucleic acids, and a SLC26A9 promoter. Representative SLC26A7 nucleic acids of the present invention are set forth as SEQ ID NOs:1 and 3, which encode SLC26A7 polypeptides set forth as SEQ ID NOs:2 and 4, respectively. Representative SLC26A9 nucleic acids of the present invention are set forth as SEQ ID NOs:5, 7, and 9, which encode SLC26A9 polypeptides set forth as SEQ ID NOs:6, 8, and 10, respectively.

As disclosed further herein below, the present invention also provides a system for functional expression of a SLC26A7 or SLC26A9 polypeptide. The system employs a recombinant SLC26 nucleic acid, including any one of odd-numbered SEQ ID NOs:1-9.

II.A. SLC26 Nucleic Acids

The terms “nucleic acid molecule” and “nucleic acid” each refer to deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms “nucleic acid molecule” or “nucleic acid” can also be used in place of “gene,” “cDNA,” “mRNA,” or “cRNA.” Nucleic acids can be synthesized, or can be derived from any biological source, including any organism. Representative methods for cloning a full-length SLC26 cDNA are described in Examples 1-2.

The terms “SLC26” and terms including “SLC26” (e.g., SLC26A7 and SLC26A9) are used herein to refer to nucleic acids that encode a SLC26 polypeptide. Thus, the term “SLC26” refers to isolated nucleic acids of the present invention comprising: (a) a nucleotide sequence comprising the nucleotide sequence of any one of odd-numbered SEQ ID NOs:1-9; or (b) a nucleotide sequence substantially identical to any one of odd-numbered SEQ ID NOs:1-9.

The term “substantially identical”, as used herein to describe a degree of similarity between nucleotide sequences, refers to two or more sequences that have at least about least 60%, preferably at least about 70%, more preferably at least about 80%, more preferably about 90% to about 99%, still more preferably about 95% to about 99%, and most preferably about 99% nucleotide identity, when compared and aligned for maximum correspondence, as measured using one of the following sequence comparison algorithms or by visual inspection. Preferably, the substantial identity exists in nucleotide sequences of at least about 100 residues, more preferably in nucleotide sequences of at least about 150 residues, and most preferably in nucleotide sequences comprising a full length coding sequence. The term “full length” is used herein to refer to a complete open reading frame encoding a functional SLC26 polypeptide, as described further herein below. Methods for determining percent identity between two polypeptides are defined herein below under the heading “Nucleotide and Amino Acid Sequence Comparisons”.

In one aspect, substantially identical sequences can be polymorphic sequences. The term “polymorphic” refers to the occurrence of two or more genetically determined alternative sequences or alleles in a population. An allelic difference can be as small as one base pair.

In another aspect, substantially identical sequences can comprise mutagenized sequences, including sequences comprising silent mutations. A mutation can comprise one or more residue changes, a deletion of residues, or an insertion of additional residues.

Another indication that two nucleotide sequences are substantially identical is that the two molecules hybridize specifically to or hybridize substantially to each other under stringent conditions. In the context of nucleic acid hybridization, two nucleic acid sequences being compared can be designated a “probe” and a “target.” A “probe” is a reference nucleic acid molecule, and a “target” is a test nucleic acid molecule, often found within a heterogeneous population of nucleic acid molecules. A “target sequence” is synonymous with a “test sequence.”

A preferred nucleotide sequence employed for hybridization studies or assays includes probe sequences that are complementary to or mimic at least an about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of any one of odd-numbered SEQ ID NOs:1-9. Such fragments can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization. “Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes, part I chapter 2, Elsevier, New York, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds (1989) Molecular Cloning: A Laboratory Manual, Cold Spring Harbor Laboratory Press, Cold Spring Harbor, N.Y. for a description of SSC buffer. Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1 M Na⁺ ion, typically about 0.01 to 1 M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to identify nucleotide sequences that are substantially identical to reference nucleotide sequences of the present invention: a probe nucleotide sequence preferably hybridizes to a target nucleotide sequence in 7% sodium dodecyl sulphate (SDS), 0.5M NaPO₄, 1 mM EDTA at 50° C. followed by washing in 2×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.5×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further under the heading “SLC26 Polypeptides” herein below. Nucleic acid molecules that do not hybridize to each other under stringent conditions are still substantially identical if the corresponding proteins are substantially identical. This can occur, for example, when two nucleotide sequences comprise conservatively substituted variants as permitted by the genetic code.

The term “conservatively substituted variants” refers to nucleic acid sequences having degenerate codon substitutions wherein the third position of one or more selected (or all) codons is substituted with mixed-base and/or deoxyinosine residues. See Batzer et al. (1991) Nucleic Acids Res 19:5081; Ohtsuka et al. (1985) J Biol Chem 260:2605-2608; and Rossolini et al. (1994) Mol Cell Probes 8:91-98.

The term “SLC26” also encompasses nucleic acids comprising subsequences and elongated sequences of a SLC26 nucleic acid, including nucleic acids complementary to a SLC26 nucleic acid, SLC26 RNA molecules, and nucleic acids complementary to SLC26 RNAs (cRNAs).

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences,” as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison methods set forth below, or is defined as being capable of hybridizing to the nucleic acid segment in question under relatively stringent conditions such as those described herein. A particular example of a complementary nucleic acid segment is an antisense oligonucleotide.

The present invention also provides chimeric genes comprising the disclosed SLC26 nucleic acids and recombinant SLC26 nucleic acids. Thus, also included are constructs and vectors comprising SLC26 nucleic acids.

The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “chimeric gene,” as used herein, refers to a promoter region operatively linked to a SLC26 sequence, including a SLC26 cDNA, a SLC26 nucleic acid encoding an antisense RNA molecule, a SLC26 nucleic acid encoding an RNA molecule having tertiary structure (e.g., a hairpin structure) or a SLC26 nucleic acid encoding a double-stranded RNA molecule. The term “chimeric gene” also refers to a SLC26 promoter region operatively linked to a heterologous sequence.

The term “operatively linked”, as used herein, refers to a functional combination between a promoter region and a nucleotide sequence such that the transcription of the nucleotide sequence is controlled and regulated by the promoter region. Techniques for operatively linking a promoter region to a nucleotide sequence are known in the art.

The term “recombinant” generally refers to an isolated nucleic acid that is replicable in a non-native environment. Thus, a recombinant nucleic acid can comprise a non-replicable nucleic acid in combination with additional nucleic acids, for example vector nucleic acids, that enable its replication in a host cell.

The term “vector” is used herein to refer to a nucleic acid molecule having nucleotide sequences that enable its replication in a host cell. A vector can also include nucleotide sequences to permit ligation of nucleotide sequences within the vector, wherein such nucleotide sequences are also replicated in a host cell. Representative vectors include plasmids, cosmids, and viral vectors. A vector can also mediate recombinant production of a SLC26 polypeptide, as described further herein below.

The term “construct”, as used herein to describe a type of construct comprising an expression construct, refers to a vector further comprising a nucleotide sequence operatively inserted with the vector, such that the nucleotide sequence is recombinantly expressed.

The terms “recombinantly expressed” or “recombinantly produced” are used interchangeably to refer generally to the process by which a polypeptide encoded by a recombinant nucleic acid is produced.

Thus, preferably recombinant SLC26 nucleic acids comprise heterologous nucleic acids. The term “heterologous nucleic acids” refers to a sequence that originates from a source foreign to an intended host cell or, if from the same source, is modified from its original form. A heterologous nucleic acid in a host cell can comprise a nucleic acid that is endogenous to the particular host cell but has been modified, for example by mutagenesis or by isolation from native cis-regulatory sequences. A heterologous nucleic acid also includes non-naturally occurring multiple copies of a native nucleotide sequence. A heterologous nucleic acid can also comprise a nucleic acid that is incorporated into a host cell's nucleic acids at a position wherein such nucleic acids are not ordinarily found.

Nucleic acids of the present invention can be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions are also known in the art. See e.g., Sambrook et al. (eds.) (1989) Molecular Cloning: A Laboratory Manual. Cold Spring Harbor Laboratory Press, Cold Spring Harbor, N.Y.; Silhavy et al. (1984) Experiments with Gene Fusions. Cold Spring Harbor Laboratory Press, Cold Spring Harbor, N.Y.; Glover & Hames (1995) DNA Cloning: A Practical Approach, 2nd ed. IRL Press at Oxford University Press, Oxford/New York; Ausubel (ed.) (1995) Short Protocols in Molecular Biology, 3rd ed. Wiley, New York.

II.B. SLC26 Polypeptides

The present invention provides novel SLC26A7 polypeptides and SLC26A9 polypeptides. Representative embodiments are set forth as even-numbered SEQ ID NOs:2-10. Preferably, an isolated SLC26 polypeptide of the present invention comprises a recombinantly expressed SLC26 polypeptide. Also preferably, isolated SLC26 polypeptides comprise functional SLC26 polypeptides.

Thus, novel SLC26 polypeptides useful in the methods of the present invention comprise: (a) a polypeptide encoded by a nucleic acid of any one of odd-numbered SEQ ID NOs:1-9; (b) a polypeptide encoded by a nucleic acid substantially identical to any one of odd-numbered SEQ ID NOs:1-9; (c) a polypeptide comprising an amino acid sequence of any one of even-numbered SEQ ID NOs:2-10; or (d) a polypeptide substantially identical to any one of even-numbered SEQ ID NOs:2-10.

The term “substantially identical”, as used herein to describe a level of similarity between SLC26 and a protein substantially identical to a SLC26 protein, refers to a sequence that is at least about 35% identical to any of even-numbered SEQ ID NOs:2-10, when compared over the full length of a SLC26 protein. Preferably, a protein substantially identical to a SLC26 protein comprises an amino acid sequence that is at least about 35% to about 45% identical to any one of even-numbered SEQ ID NOs:2-10, more preferably at least about 45% to about 55% identical to any one of even-numbered SEQ ID NOs:2-10, even more preferably at least about 55% to about 65% identical to any one of even-numbered SEQ ID NOs:2-10, still more preferably preferably at least about 65% to about 75% identical to any one of even-numbered SEQ ID NOs:2-10, still more preferably preferably at least about 75% to about 85% identical to any one of even-numbered SEQ ID NOs:2-10, still more preferably preferably at least about 85% to about 95% identical to any one of even-numbered SEQ ID NOs:2-10, and still more preferably at least about 95% to about 99% identical to any one of even-numbered SEQ ID NOs:2-10 when compared over the full length of a SLC26 polypeptide. The term “full length” refers to a functional SLC26 polypeptide, as described further herein below. Methods for determining percent identity between two polypeptides are also defined herein below under the heading “Nucleotide and Amino Acid Sequence Comparisons”.

The term “substantially identical,” when used to describe polypeptides, also encompasses two or more polypeptides sharing a conserved three-dimensional structure. Computational methods can be used to compare structural representations, and structural models can be generated and easily tuned to identify similarities around important active sites or ligand binding sites. See Saqi et al. (1999) Bioinformatics 15:521-522; Barton (1998) Acta Crystallogr D Biol Crystallogr 54:1139-1146; Henikoff et al. (2000) Electrophoresis 21:1700-1706; and Huang et al. (2000) Pac Symp Biocomput:230-241.

Substantially identical proteins also include proteins comprising amino acids that are functionally equivalent to amino acids of any one of even-numbered SEQ ID NOs:2-10. The term “functionally equivalent” in the context of amino acids is known in the art and is based on the relative similarity of the amino acid side-chain substituents. See Henikoff & Henikoff (2000) Adv Protein Chem 54:73-97. Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al., 1982). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those which are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0 μl); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within ±0.5 of the original value are even more particularly preferred.

The term “substantially identical” also encompasses polypeptides that are biologically functional equivalents of a SLC26 polypeptide. The term “functional” includes an activity of an SLC26 polypeptide in transporting anions across a membrane. Preferably, such transport shows a magnitude and anion selectivity that is substantially similar to that of a cognate SLC26 polypeptide in vivo. Preferably, the term “functional” also refers to similar kinetics of activation and inactivation of anion transport activity. Representative methods for assessing anion transport activity are described herein below.

The present invention also provides functional fragments of a SLC26 polypeptide. Such functional portion need not comprise all or substantially all of the amino acid sequence of a native SLC26 gene product.

The present invention also includes functional polypeptide sequences that are longer sequences than that of a native SLC26 polypeptide. For example, one or more amino acids can be added to the N-terminus or C-terminus of a SLC26 polypeptide. Such additional amino acids can be employed in a variety of applications, including but not limited to purification applications. Methods of preparing elongated proteins are known in the art.

II.C. Nucleotide and Amino Acid Sequence Comparisons

The terms “identical” or “percent identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain biological function of a SLC26 nucleic acid or a SLC26 polypeptide.

For comparison of two or more sequences, typically one sequence acts as a reference sequence to which one or more test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, for example, by the local homology algorithm of Smith & Waterman (1981) Adv Appl Math 2:82-489, by the homology alignment algorithm of Needleman & Wunsch (1970) J Mol Biol 48:443-453, by the search for similarity method of Pearson & Lipman (1988) Proc Natl Acad Sci USA 85:2444-2448, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel (ed.) (1995) Short Protocols in Molecular Biology, 3rd ed. Wiley, New York.

A preferred algorithm for determining percent sequence identity and sequence similarity is the BLAST algorithm, which is described in Altschul et al. (1990) J Mol Biol 215:403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff (1992) Proc Natl Acad Sci USA 89:10915-10919.

In addition to calculating percent sequence identity, the BLAST algorithm also performs a statistical analysis of the similarity between two sequences. See e.g., Karlin & Altschul (1993) Proc Natl Acad Sci USA 90:5873-5877. One measure of similarity provided by the BLAST algorithm is the smallest sum probability (P(N)), which provides an indication of the probability by which a match between two nucleotide or amino acid sequences that would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

III. Methods for Detecting a SLC26 Nucleic Acid In another aspect of the invention, a method is provided for detecting a nucleic acid molecule that encodes a SLC26 polypeptide. Such methods can be used to detect SLC26 gene variants or altered gene expression. For example, detection of a change in SLC26 sequence or expression can be used for diagnosis of SLC26-related diseases, disorders, and drug interactions. Preferably, the nucleic acids used for this method comprise sequences set forth as any one of odd-numbered SEQ ID NOs:1-9.

Sequences detected by methods of the invention can detected, subcloned, sequenced, and further evaluated by any measure well known in the art using any method usually applied to the detection of a specific DNA sequence. Thus, the nucleic acids of the present invention can be used to clone genes and genomic DNA comprising the disclosed sequences. Alternatively, the nucleic acids of the present invention can be used to clone genes and genomic DNA of related sequences. Using the nucleic acid sequences disclosed herein, such methods are known to one skilled in the art. See e.g., Sambrook et al., eds (1989) Molecular Cloning, Cold Spring Harbor Laboratory Press, Cold Spring Harbor, N.Y. Representative methods are also disclosed in Examples 1-5.

In one embodiment of the invention, levels of a SLC26 nucleic acid molecule are measured by, for example, using an RT-PCR assay. See Chiang (1998) J Chromatogr A 806:209-218, and references cited therein.

In another embodiment of the invention, genetic assays based on nucleic acid molecules of the present invention can be used to screen for genetic variants, for example by allele-specific oligonucleotide (ASO) probe analysis (Conner et al., 1983), oligonucleotide ligation assays (OLAs) (Nickerson et al., 1990), single-strand conformation polymorphism (SSCP) analysis (Orita et al., 1989), SSCP/heteroduplex analysis, enzyme mismatch cleavage, direct sequence analysis of amplified exons (Kestila et al., 1998; Yuan et al., 1999), allele-specific hybridization (Stoneking et al., 1991), and restriction analysis of amplified genomic DNA containing the specific mutation. Automated methods can also be applied to large-scale characterization of single nucleotide polymorphisms (Wang et al., 1998; Brookes, 1999). Preferred detection methods are non-electrophoretic, including, for example, the TAQMAN™ allelic discrimination assay, PCR-OLA, molecular beacons, padlock probes, and well fluorescence. See Landegren et al. (1998) Genome Res 8:769-776 and references cited therein.

IV. System for Recombinant Expression of a SLC26 Polypeptide

The present invention further provides a system for expression of a recombinant SLC26 polypeptide of the present invention. Such a system can be used for subsequent purification and/or characterization of a SLC26 polypeptide. For example, a purified SLC26A7 or SLC26A9 polypeptide can be used as an immunogen for the production of an SLC26 antibody, described further herein below.

A system for recombinant expression of a SLC26 polypeptide can also be used for the identification of modulators of anion transport. In one embodiment of the invention, a method is provided for identification of SLC26 modulators, as described herein below. Alternatively, the disclosed SLC26 polypeptides can be used as a control anion transporter when testing any other molecule for anion transport activity. For example, the present invention discloses that SLC26A7 and SLC26A9 are chloride transporters, and thus a system for recombinant SLC26A7 or SLC26A9 expression can be used as a positive control in an assay to determine chloride transport of a test polypeptide. Such test polypeptides can include candidates for any one of a variety of hereditary and acquired disease such as cystic fibrosis, nephrolithiasis, and cholera.

The term “expression system” refers to a host cell comprising a heterologous nucleic acid and the polypeptide encoded by the heterologous nucleic acid. For example, a heterologous expression system can comprise a host cell transfected with a construct comprising a recombinant SLC26 nucleic acid, a host cell transfected with SLC26 cRNA, or a cell line produced by introduction of heterologous nucleic acids into a host cell genome.

A system for recombinant expression of a SLC26 polypeptide can comprise: (a) a recombinantly expressed SLC26 polypeptide; and (b) a host cell comprising the recombinantly expressed SLC26 polypeptide. For example, a SLC26 cRNA can be transcribed in vitro and then introduced into a host cell, whereby a SLC26 polypeptide is expressed. In a preferred embodiment of the invention, SLC26 cRNA is provided to a host cell by direct injection of a solution comprising the SLC26 cRNA, as described in Example 6. The system can further comprise a plurality of different SLC26 polypeptides.

A system for recombinant expression of a SLC26 polypeptide can also comprise: (a) a construct comprising a vector and a nucleic acid molecule encoding a SLC26 polypeptide operatively linked to a heterologous promoter; and (b) a host cell comprising the construct of (a), whereby the host cell expresses a SLC26 polypeptide. The system can further comprise constructs encoding a plurality of different SLC26 polypeptides. Additionally, a single construct itself can encode a plurality of different SLC26 polypeptides.

Isolated polypeptides and recombinantly produced polypeptides can be purified and characterized using a variety of standard techniques that are known to the skilled artisan. See e.g., Schröder & Lübke (1965) The Peptides. Academic Press, New York; Schneider & Eberle (1993) Peptides, 1992: Proceedings of the Twenty-Second European Peptide Symposium, Sep. 13-19, 1992, Interlaken, Switzerland. Escom, Leiden; Bodanszky (1993) Principles of Peptide Synthesis, 2nd rev. ed. Springer-Verlag, Berlin; New York; Ausubel (ed.) (1995) Short Protocols in Molecular Biology, 3rd ed. Wiley, New York.

Preferably, a recombinantly expressed SLC26 polypeptide comprises a functional anion transporter. Thus, a recombinantly expressed SLC26 polypeptide preferably displays transport of Cl⁻, SO₄²⁻, oxalate, and/or formate across a lipid bilayer or membrane. Also preferably, a recombinant SLC26 polypeptide shows ion selectivity similar to a native SLC26 polypeptide. Representative methods for determining SLC26 function are described herein below.

IV.A. Expression Constructs

A construct for expression of a SLC26 polypeptide includes a vector and a SLC26 nucleotide sequence, wherein the SLC26 nucleotide sequence is operatively linked to a promoter sequence. A construct for recombinant SLC26 expression can also comprise transcription termination signals and sequences required for proper translation of the nucleotide sequence. Preparation of an expression construct, including addition of translation and termination signal sequences, is known to one skilled in the art.

Recombinant production of a SLC26 polypeptide can be directed using a constitutive promoter or an inducible promoter. Representative promoters that can be used in accordance with the present invention include Simian virus 40 early promoter, a long terminal repeat promoter from retrovirus, an actin promoter, a heat shock promoter, and a metallothien protein.

Suitable vectors that can be used to express a SLC26 polypeptide include but are not limited to viruses such as vaccinia virus or adenovirus, baculovirus vectors, yeast vectors, bacteriophage vectors (e.g., lambda phage), plasmid and cosmid DNA vectors, transposon-mediated transformation vectors, and derivatives thereof.

Constructs are introduced into a host cell using a transfection method compatible with the vector employed. Standard transfection methods include electroporation, DEAE-Dextran transfection, calcium phosphate precipitation, liposome-mediated transfection, transposon-mediated transformation, infection using a retrovirus, particle-mediated gene transfer, hyper-velocity gene transfer, and combinations thereof.

IV.B. Host Cells

The term “host cell”, as used herein, refers to a cell into which a heterologous nucleic acid molecule can be introduced. Any suitable host cell can be used, including but not limited to eukaryotic hosts such as mammalian cells (e.g., HeLa cells, CV-1 cells, COS cells), amphibian cells (e.g., Xenopus oocytes), insect cells (e.g., Sf9 cells), as well as prokaryotic hosts such as E. coli and Bacillus subtilis. Preferred host cells are amphibian cells such as Xenopus oocytes. Also preferably, a host cell substantially lacks a SLC26 polypeptide.

A host cell strain can be chosen which modulates the expression of the recombinant sequence, or modifies and processes the gene product in the specific fashion desired. For example, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system can be used to produce a non-glycosylated core protein product, and expression in yeast will produce a glycosylated product.

The present invention further encompasses recombinant expression of a SLC26 polypeptide in a stable cell line. Methods for generating a stable cell line following transformation of a heterologous construct into a host cell are known in the art. See e.g., Joyner (1993) Gene Targeting: A Practical Approach. Oxford University Press, Oxford/New York. Thus, transformed cells, tissues, or non-human organisms are understood to encompass not only the end product of a transformation process, but also transgenic progeny or propagated forms thereof.

The present invention further encompasses cryopreservation of cells expressing a recombinant SLC26 polypeptide as disclosed herein. Thus, transiently transfected cells and cells of a stable cell line expressing SLC26 can be frozen and stored for later use. Frozen cells can be readily transported for use at a remote location.

Cryopreservation media generally consists of a base medium, cryopreservative, and a protein source. The cryopreservative and protein protect the cells from the stress of the freeze-thaw process. For serum-containing medium, a typical cryopreservation medium is prepared as complete medium containing 10% glycerol; complete medium containing 10% DMSO (dimethylsulfoxide), or 50% cell-conditioned medium with 50% fresh medium with 10% glycerol or 10% DMSO. For serum-free medium, typical cryopreservation formulations include 50% cell-conditioned serum free medium with 50% fresh serum-free medium containing 7.5% DMSO; or fresh serum-free medium containing 7.5% DMSO and 10% cell culture grade DMSO. Preferably, a cell suspension comprising about 10⁶ to about 10⁷ cells per ml is mixed with cryopreservation medium.

Cells are combined with cryopreservation medium in a vial or other container suitable for frozen storage, for example NUNC® CRYOTUBES™ (available from Applied Scientific of South San Francisco, Calif.). Cells can also be aliquotted to wells of a multi-well plate, for example a 96-well plate designed for high-throughput assays, and frozen in plated format.

Cells are preferably cooled from room temperature to a storage temperature at a rate of about −1° C. per minute. The cooling rate can be controlled, for example, by placing vials containing cells in an insulated water-filled reservoir having about 1 liter liquid capacity, and placing such cube in a −70° C. mechanical freezer. Alternatively, the rate of cell cooling can be controlled at about −1° C. per minute by submersing vials in a volume of liquid refrigerant such as an aliphatic alcohol, the volume of liquid refrigerant being more than fifteen times the total volume of cell culture to be frozen, and placing the submersed culture vials in a conventional freezer at a temperature below about −70° C. Commercial devices for freezing cells are also available, for example, the Planer Mini-Freezer R202/200R (Planer Products Ltd. of Great Britain) and the BF-5 Biological Freezer (Union Carbide Corporation of Danbury, Conn., United States of America). Preferably, frozen cells are stored at or below about −70° C. to about −80° C., and more preferably at or below about −130° C.

To obtain the best possible cell survival, thawing of the cells must be performed as quickly as possible. Once a vial or other reservoir containing frozen cells is removed from storage, it should be placed directly into a 37° C. water bath and gently shaken until it is completely thawed. If cells are particularly sensitive to cryopreservatives, the cells are centrifuged to remove cryopreservative prior to further growth.

Additional methods for preparation and handling of frozen cells can be found in Freshney (1987) Culture of Animal Cells: A Manual of Basic Technique, 2nd ed. A. R. Liss, New York and in U.S. Pat. Nos. 6,176,089; 6,140,123; 5,629,145; and 4,455,842; among other places.

V. Transgenic Animals

The present invention also provides a transgenic animal comprising a disruption of SLC26A7 or SLC26A9 gene expression. Altered gene expression can include expression of an altered level or mutated variant of a SLC26A7 or SLC26A9 gene. The present invention provides nucleic acids encoding SLC26A7 and SLC26A9 that can be used to prepare constructs for generating a transgenic animal. Also provided is genomic localization data useful for preparation of constructs targeted to the SLC26A7 or SLC26A9 locus.

In one embodiment of the present invention, the transgenic animal can comprise a mouse with targeted modification of the mouse SLC26A7 or SLC26A9 locus and can further comprise mice strains with complete or partial functional inactivation of the SLC26A7 or SLC26A9 genes in all somatic cells.

In an alternative embodiment, a transgenic animal in accordance with the present invention is prepared using anti-sense or ribozyme SLC26A7 or SLC26A9 constructs, driven by a universal or tissue-specific promoter, to reduce levels of SLC26 gene expression in somatic cells, thus achieving a “knock-down” phenotype. The present invention also provides the generation of murine strains with conditional or inducible inactivation of SLC26A7, SLC26A9, or a combination thereof. Such murine strains can also comprise additional synthetic or naturally occurring mutations, for example a mutation in any other SLC26 gene.

The present invention also provides mice strains with specific “knocked-in” modifications in the SLC26A7 and SLC26A9 genes, for example to create an over-expression or dominant negative phenotype. Thus, “knocked-in” modifications include the expression of both wild type and mutated forms of a nucleic acid encoding a SLC26A7 or SLC26A9 polypeptide.

Techniques for the preparation of transgenic animals are known in the art. Exemplary techniques are described in U.S. Pat. No. 5,489,742 (transgenic rats); U.S. Pat. Nos. 4,736,866, 5,550,316, 5,614,396, 5,625,125 and 5,648,061 (transgenic mice); U.S. Pat. No. 5,573,933 (transgenic pigs); 5,162,215 (transgenic avian species) and U.S. Pat. No. 5,741,957 (transgenic bovine species), the entire contents of each of which are herein incorporated by reference.

For example, a transgenic animal of the present invention can comprises a mouse with targeted modification of the mouse SLC26A7 or SLC26A9 gene. Mice strains with complete or partial functional inactivation of the SLC26A7 or SLC26A9 genes in all somatic cells are generated using standard techniques of site-specific recombination in murine embryonic stem cells. See Capecchi, M. R. (1989) Science 244(4910): 1288-92; Thomas, K. R., and Capecchi, M. R. (1990) Nature 346(6287): 847-50; Delpire, E., et al. (1999) Nat Genet 22(2): 192-5.

VI. SLC26 Antibodies

In another aspect of the invention, a method is provided for producing an antibody that specifically binds a SLC26 polypeptide. According to the method, a full-length recombinant SLC26 polypeptide, or fragment thereof, is formulated so that it can be used as an effective immunogen, and used to immunize an animal so as to generate an immune response in the animal. The immune response is characterized by the production of antibodies that can be collected from the blood serum of the animal. The present invention also provides antibodies produced by methods that employ the novel SLC26 polypeptides disclosed herein, including any one of even-numbered SEQ ID NOs:2-10.

The term “antibody” refers to an immunoglobulin protein, or functional portion thereof, including a polyclonal antibody, a monoclonal antibody, a chimeric antibody, a hybrid antibody, a single chain antibody, a mutagenized antibody, a humanized antibody, and antibody fragments that comprise an antigen binding site (e.g., Fab and Fv antibody fragments). In a preferred embodiment of the invention, a SLC26 antibody comprises a monoclonal antibody. Thus, the present invention also encompasses antibodies and cell lines that produce monoclonal antibodies as described herein.

The term “specifically binds”, when used to describe binding of an antibody to a SLC26 polypeptide, refers to binding to a SLC26 polypeptide in a heterogeneous mixture of other polypeptides.

The phrases “substantially lack binding” or “substantially no binding”, as used herein to describe binding of an antibody to a control polypeptide or sample, refers to a level of binding that encompasses non-specific or background binding, but does not include specific binding.

Techniques for preparing and characterizing antibodies are known in the art. See e.g., Harlow & Lane (1988) Antibodies: A Laboratory Manual, Cold Spring Harbor Laboratory Press, Cold Spring Harbor, N.Y. and U.S. Pat. Nos. 4,196,265; 4,946,778; 5,091,513; 5,132,405; 5,260,203; 5,677,427; 5,892,019; 5,985,279; 6,054,561.

SLC26 antibodies prepared as disclosed herein can be used in methods known in the art relating to the localization and activity of SLC26 polypeptides, e.g., for cloning of nucleic acids encoding a SLC26 polypeptide, immunopurification of a SLC26 polypeptide, imaging a SLC26 polypeptide in a biological sample, and measuring levels of a SLC26 polypeptide in appropriate biological samples. To perform such methods, an antibody of the present invention can further comprise a detectable label, including but not limited to a radioactive label, a fluorescent label, an epitope label, and a label that can be detected in vivo. Methods for selection of a label suitable for a particular detection technique, and methods for conjugating to or otherwise associating a detectable label with an antibody are known to one skilled in the art.

VII. SLC26 Modulators

The present invention further discloses assays to identify modulators of SLC26 activity. An assay can employ a system for expression of a SLC26 polypeptide, as disclosed herein above, or an isolated SLC26 polypeptide produced in such a system. The present invention also provides modulators of anion transport activity identified using the disclosed methods.

The term “modulate” means an increase, decrease, or other alteration of any or all chemical and biological activities or properties of a SLC26 polypeptide. Thus, the method for identifying modulators involves assaying a level or quality of SLC26 function.

A method for identifying a modulator of anion transport can comprise: (a) providing a recombinant expression system whereby a SLC26 polypeptide is expressed in a host cell, and wherein the SLC26 polypeptide comprises a SLC26A7 polypeptide or a SLC26A9 polypeptide; (b) providing a test substance to the system of (a); (c) assaying a level or quality of SLC26 function in the presence of the test substance; (d) comparing the level or quality of SLC26 function in the presence of the test substance with a control level or quality of SLC26 function; and (e) identifying a test substance as an anion transport modulator by determining a level or quality of SLC26 function in the presence of the test substance as significantly changed when compared to a control level or quality of SLC26 function.

In one embodiment of the invention, assaying SLC26 function comprises determining a level of SLC26 gene expression.

In another embodiment of the invention, assaying SLC26 function comprises assaying binding activity of a recombinantly expressed SLC26 polypeptide. For example, a SLC26 activity can comprise an amount or a strength of binding of a modulator to a SLC26 polypeptide.

In still another embodiment of the invention, assaying SLC26 function can comprise assaying an active conformation of a SLC26 polypeptide.

In a preferred embodiment of the invention, assaying SLC26 function comprises assaying anion transport activity of a recombinantly expressed SLC26 polypeptide. A representative level of SLC26 activity can thus comprise an amount of anion transport or a peak level of anion transport, measurable as described in Example 7. A representative quality of SLC26 activity can comprise, for example, anion selectivity of a SLC26 polypeptide, pH sensitivity of anion transport, and pharmacological sensitivity of a SLC26 polypeptide. The electrophysiological behavior of SLC26A6 and other SLC26 polypeptides also provides a signature for transport activity.

A control level or quality of SLC26 activity refers to a level or quality of wild type SLC26 activity. Preferably, a system for recombinant expression of a SLC26 polypeptide comprises any one of even-numbered SEQ ID NOs:2-10. When evaluating the modulating capacity of a test substance, a control level or quality of SLC26 activity comprises a level or quality of activity in the absence of a test substance.

The term “significantly changed”, as used herein to refer to an altered level or activity of a SLC26 polypeptide, refers to a quantified change in a measurable quality that is larger than the margin of error inherent in the measurement technique, preferably an increase or decrease by about 2-fold or greater relative to a control measurement, more preferably an increase or decrease by about 5-fold or greater, and most preferably an increase or decrease by about 10-fold or greater.

Modulators identified by the disclosed methods can comprise agonists and antagonists. As used herein, the term “agonist” means a substance that activates, synergizes, or potentiates the biological activity of a SLC26 polypeptide. As used herein, the term “antagonist” refers to a substance that blocks or mitigates the biological activity of a SLC26 polypeptide. A modulator can also comprise a ligand or a substance that specifically binds to a SLC26 polypeptide. Activity and binding assays for the determination of a SLC26 modulator can be performed in vitro or in vivo.

In one embodiment of the invention, such assays are useful for the identification of SLC26 modulators that can be developed for the treatment and/or diagnosis of SLC26-related disorders, as described further herein below under the heading “Therapeutic Applications.”

In another embodiment of the invention, assays using a recombinant SLC26 polypeptide can be performed for the purpose of prescreening bioactive agents, wherein an interaction between the agent and SLC26 is undesirable. Thus, drugs intended for administration to a subject for the treatment of a non-SLC26-related disorder can be tested for SLC26 modulating activity that can result in undesirable side effects. The disclosed assays and methods enable pre-screening of bioactive agents under development to identify deleterious effects of anion transport.

In still another embodiment of the invention, an assay disclosed herein can be used to characterize a mutant SLC26 polypeptide, for example a mutant polypeptide that is linked to a disorder of anion transport. Recombinant expression of mutated SLC26 polypeptides will permit further analysis of disorder-related SLC26 anion transporters.

In accordance with the present invention there is also provided a rapid and high throughput screening method that relies on the methods described herein. This screening method comprises separately contacting a SLC26 polypeptide with a plurality of test substances. In such a screening method the plurality of target substances preferably comprises more than about 10⁴ samples, or more preferably comprises more than about 10⁵ samples, and still more preferably more than about 10⁶ samples.

VII.A. Test Substances

A potential modulator assayed using the methods of the present invention comprises a candidate substance. As used herein, the terms “candidate substance” and “test substance” are used interchangeably, and each refers to a substance that is suspected to interact with a SLC26 polypeptide, including any synthetic, recombinant, or natural product or composition. A test substance suspected to interact with a polypeptide can be evaluated for such an interaction using the methods disclosed herein.

Representative test substances include but are not limited to peptides, oligomers, nucleic acids (e.g., aptamers), small molecules (e.g., chemical compounds), antibodies or fragments thereof, nucleic acid-protein fusions, any other affinity agent, and combinations thereof. A test substance can additionally comprise a carbohydrate, a vitamin or derivative thereof, a hormone, a neurotransmitter, a virus or receptor binding domain thereof, an opsin or rhodopsin, an odorant, a phermone, a toxin, a growth factor, a platelet activation factor, a neuroactive peptide, or a neurohormone. A candidate substance to be tested can be a purified molecule, a homogenous sample, or a mixture of molecules or compounds.

The term “small molecule” as used herein refers to a compound, for example an organic compound, with a molecular weight of less than about 1,000 daltons, more preferably less than about 750 daltons, still more preferably less than about 600 daltons, and still more preferably less than about 500 daltons. A small molecule also preferably has a computed log octanol-water partition coefficient in the range of about −4 to about +14, more preferably in the range of about −2 to about +7.5.

Test substances can be obtained or prepared as a library. As used herein, the term “library” means a collection of molecules. A library can contain a few or a large number of different molecules, varying from about ten molecules to several billion molecules or more. A molecule can comprise a naturally occurring molecule, a recombinant molecule, or a synthetic molecule. A plurality of test substances in a library can be assayed simultaneously. Optionally, test substances derived from different libraries can be pooled for simultaneous evaluation.

Representative libraries include but are not limited to a peptide library (U.S. Pat. Nos. 6,156,511, 6,107,059, 5,922,545, and 5,223,409), an oligomer library (U.S. Pat. Nos. 5,650,489 and 5,858,670), an aptamer library (U.S. Pat. Nos. 6,180,348 and 5,756,291), a small molecule library (U.S. Pat. Nos. 6,168,912 and 5,738,996), a library of antibodies or antibody fragments (U.S. Pat. Nos. 6,174,708, 6,057,098, 5,922,254, 5,840,479, 5,780,225, 5,702,892, and 5,667,988), a library of nucleic acid-protein fusions (U.S. Pat. No. 6,214,553), and a library of any other affinity agent that can potentially bind to a SLC26 polypeptide (e.g., U.S. Pat. Nos. 5,948,635, 5,747,334, and 5,498,538).

A library can comprise a random collection of molecules. Alternatively, a library can comprise a collection of molecules having a bias for a particular sequence, structure, or conformation. See e.g., U.S. Pat. Nos. 5,264,563 and 5,824,483. Methods for preparing libraries containing diverse populations of various types of molecules are known in the art, for example as described in U.S. Patents cited herein above. Numerous libraries are also commercially available.

VII.B. Binding Assays

In another embodiment, a method for identifying of a SLC26 modulator comprises determining specific binding of a test substance to a SLC26 polypeptide. The term “binding” refers to an affinity between two molecules. Preferably, specific binding also encompasses a quality or state of mutual action such that an activity of one protein or compound on another protein is inhibitory (in the case of an antagonist) or enhancing (in the case of an agonist).

The phrase “specifically (or selectively) binds”, when referring to the binding capacity of a candidate modulator, refers to a binding reaction which is determinative of the presence of the protein in a heterogeneous population of proteins and other biological materials. The binding of a modulator to a SLC26 polypeptide can be considered specific if the binding affinity is about 1×10⁴M⁻¹ to about 1×10⁶M⁻¹ or greater. The phrase “specifically binds” also refers to saturable binding. To demonstrate saturable binding of a test substance to a SLC26 polypeptide, Scatchard analysis can be carried out as described, for example, by Mak et al. (1989) J Biol Chem 264:21613-21618.

The phases “substantially lack binding” or “substantially no binding”, as used herein to describe binding of a modulator to a control polypeptide or sample, refers to a level of binding that encompasses non-specific or background binding, but does not include specific binding.

Several techniques can be used to detect interactions between a SLC26 polypeptide and a test substance without employing a known competitive modulator. Representative methods include, but are not limited to, Fluorescence Correlation Spectroscopy, Surface-Enhanced Laser Desorption/Ionization Time-Of-flight Spectroscopy, and Biacore technology, each technique described herein below. These methods are amenable to automated, high-throughput screening.

Fluorescence Correlation Spectroscopy. Fluorescence Correlation Spectroscopy (FCS) measures the average diffusion rate of a fluorescent molecule within a small sample volume (Tallgren, 1980). The sample size can be as low as 10³ fluorescent molecules and the sample volume as low as the cytoplasm of a single bacterium. The diffusion rate is a function of the mass of the molecule and decreases as the mass increases. FCS can therefore be applied to polypeptide-ligand interaction analysis by measuring the change in mass and therefore in diffusion rate of a molecule upon binding. In a typical experiment, the target to be analyzed (e.g., a SLC26 polypeptide) is expressed as a recombinant polypeptide with a sequence tag, such as a poly-histidine sequence, inserted at the N-terminus or C-terminus. The expression is mediated in a host cell, such as E. coli, yeast, Xenopus oocytes, or mammalian cells. The polypeptide is purified using chromatographic methods. For example, the poly-histidine tag can be used to bind the expressed polypeptide to a metal chelate column such as Ni²⁺ chelated on iminodiacetic acid agarose. The polypeptide is then labeled with a fluorescent tag such as carboxytetramethylrhodamine or BODIPY™ reagent (available from Molecular Probes of Eugene, Oreg.). The polypeptide is then exposed in solution to the potential ligand, and its diffusion rate is determined by FCS using instrumentation available from Carl Zeiss, Inc. (Thornwood, N.Y.). Ligand binding is determined by changes in the diffusion rate of the polypeptide.

Surface-Enhanced Laser Desorption/Ionization. Surface-Enhanced Laser Desorption/Ionization (SELDI) was developed by Hutchens & Yip (1993) Rapid Commun Mass Spectrom 7:576-580. When coupled to a time-of-flight mass spectrometer (TOF), SELDI provides a technique to rapidly analyze molecules retained on a chip. It can be applied to ligand-protein interaction analysis by covalently binding the target protein, or portion thereof, on the chip and analyzing by mass spectrometry the small molecules that bind to this protein (Worrall et al., 1998). In a typical experiment, a target polypeptide (e.g., a SLC26 polypeptide) is recombinantly expressed and purified. The target polypeptide is bound to a SELDI chip either by utilizing a poly-histidine tag or by other interaction such as ion exchange or hydrophobic interaction. A chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind a target polypeptide are identified by the stringency of the wash needed to elute them.

Biacore. Biacore relies on changes in the refractive index at the surface layer upon binding of a ligand to a target polypeptide (e.g., a SLC26 polypeptide) immobilized on the layer. In this system, a collection of small ligands is injected sequentially in a 2-5 microliter cell, wherein the target polypeptide is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein (Liedberg et al., 1983; Malmquist, 1993). In a typical experiment, a target protein is recombinantly expressed, purified, and bound to a Biacore chip. Binding can be facilitated by utilizing a poly-histidine tag or by other interaction such as ion exchange or hydrophobic interaction. A chip thus prepared is then exposed to one or more potential ligands via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction. See also Homola et al. (1999) Sensors and Actuators 54:3-15 and references therein.

VII.C. Conformational Assay

The present invention also provides a method for identifying a SLC26 modulator that relies on a conformational change of a SLC26 polypeptide when bound by or otherwise interacting with a SLC26 modulator.

Application of circular dichroism to solutions of macromolecules reveals the conformational states of these macromolecules. The technique can distinguish random coil, alpha helix, and beta chain conformational states.

To identify modulators of a SLC26 polypeptide, circular dichroism analysis can be performed using a recombinantly expressed SLC26 polypeptide. A SLC26 polypeptide is purified, for example by ion exchange and size exclusion chromatography, and mixed with a test substance. The mixture is subjected to circular dichroism. The conformation of a SLC26 polypeptide in the presence of a test substance is compared to a conformation of a SLC26 polypeptide in the absence of a test substance. A change in conformational state of a SLC26 polypeptide in the presence of a test substance can thus be used to identify a SLC26 modulator. Representative methods are described in U.S. Pat. Nos. 5,776,859 and 5,780,242.

VII.D. Anion Transport Assays

In a preferred embodiment of the invention, a method for identifying a SLC26 modulator employs a functional SLC26 polypeptide. Novel SLC26 polypeptides disclosed herein include any of even-numbered SEQ ID NOs:2-10. Representative methods for determining anion transport activity of a functional SLC26 modulator include measuring anion flux and determining electrogenic transport, each described briefly herein below.

In accordance with the method, cells expressing SLC26 can be provided in the form of a kit useful for performing an assay of SLC26 function. Thus, cells can be frozen as described herein above and transported while frozen to others for performance of an assay. For example, in one embodiment of the invention, a test kit is provided for detecting a SLC26 modulator, the kit comprising: (a) frozen cells transfected with DNA encoding a full-length SLC26 polypeptide; and (b) a medium for growing the cells.

Preferably, a cell used in such an assay comprises a cell that is substantially devoid of native SLC26 and polypeptides substantially similar to SLC26. A preferred cell comprises a vertebrate cell, for example a Xenopus oocyte. In one embodiment of the invention, a cell used in the assay comprises a stable cell line that recombinantly expresses SLC26. Alternatively, a cell used in the assay can transiently express a SLC26 polypeptide as described in Example 6.

The term “substantially devoid of”, as used herein to describe a host cell or a control cell, refers to a quality of having a level of native SLC26A, a level of a polypeptide substantially similar to SLC26A, or a level of activity thereof, comprising a background level. The term “background level” encompasses non-specific measurements of expression or activity that are typically detected in a cell free of SLC26 and free of polypeptides substantially similar to a SLC26 polypeptide.

Also preferably, all assays employing cells expressing recombinant SLC26 additionally employ control cells that are substantially devoid of native SLC26 and polypeptides substantially similar to a SLC26 polypeptide. When using transiently transfected cells, a control cell can comprise, for example, an untransfected host cell. When using a stable cell line expressing a SLC26 polypeptide, a control cell can comprise, for example, a parent cell line used to derive the SLC26-expressing cell line.

Assays of SLC26 activity that employ transiently transfected cells preferably include a marker that distinguishes transfected cells from non-transfected cells. The term “marker” refers to any detectable molecule that can be used to distinguish a cell that recombinantly expresses SLC26 from a cell that does not recombinantly express a SLC26 polypeptide. Preferably, a marker is encoded by or otherwise associated with a construct for SLC26 expression, such that cells are simultaneously transfected with a nucleic acid molecule encoding SLC26 and the marker. Representative detectable molecules that are useful as markers include but are not limited to a heterologous nucleic acid, a polypeptide encoded by a transfected construct (e.g., an enzyme or a fluorescent polypeptide), a binding protein, and an antigen.

A marker comprising a heterologous nucleic acid includes nucleic acids encoding a SLC26 polypeptide. Alternatively, any suitable method can be used to detect the encoded SLC26 polypeptide, as described herein below.

Examples of enzymes that are useful as markers include phosphatases (such as acid or alkaline phosphatase), β-galactosidase, urease, glucose oxidase, carbonic anhydrase, acetylcholinesterase, glucoamylase, maleate dehydrogenase, glucose-6-phosphate dehydrogenase, β-glucosidase, proteases, pyruvate decarboxylase, esterases, luciferase, alcohol dehydrogenase, or peroxidases (such as horseradish peroxidase).

A marker comprising an enzyme can be detected based on activity of the enzyme. Thus, a substrate is be added to catalyze a reaction the end product of which is detectable, for example using spectrophotometer, a luminometer, or a fluorimeter. Substrates for reaction by the above-mentioned enzymes, and that produce a detectable reaction product, are known to one of skill in the art.

A preferred marker comprises an encoded polypeptide that can be detected in the absence of an added substrate. Representative polypeptides that can be detected directly include GFP and EGFP. Common research equipment has been developed to perform high-throughput detection of fluorescence, for example GFP or EGFP fluorescence, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), and Genomic Solutions Inc. (Ann Arbor, Mich., United States of America). Most of the commercial systems use some form of scanning technology with photomultiplier tube detection.

Anion Flux Assay. A candidate substance can be tested for its ability to modulate a SLC26 polypeptide by determining anion flux across a membrane or lipid bilayer. Anion levels can be determined by any suitable approach. For example, an anion can be detected using a radiolabeled anion as described in Example 7.

Anion flux can also be measured using any of a variety of indicator compounds. Preferably, an indicator compound comprises a compound that can be detected in a high-throughput capacity. Representative fluorescent indicators useful for detecting halides (e.g., chloride) include quinolium-type Cl⁻ indicators (Verkman, 1990; Mansoura et al., 1999), cell-permeable indicators (Biwersi & Verkman, 1991), ratiometric indicators (Biwersi & Verkman, 1991), and long wavelength indicators (Biwersi et al., 1994; Jayaraman et al., 1999). An indicator can also comprise a recombinant protein. For example, the yellow fluorescent protein mutant, YFP-H148Q, produces fluorescence that is decreased upon halide binding (Jayaraman et al., 2000; Galietta et al., 2001). Such indicators are compatible with high-throughput assay formats and can be detected using, for example, an instrument for fluorescent detection as noted herein above.

Anion flux in a population of cultured cells can also be measured based on changes in a degree of light scattering that is correlated with cell size. See e.g., Krick et al. (1998) Pflugers Arch 435:415-421.

An anion flux assay can also comprise a competitive assay design. For example, the method can comprise: (a) providing an expression system, whereby a functional SLC26 polypeptide is expressed; (b) adding a SLC26 activator to the expression system, whereby anion transport is elicited; (c) adding a test substance to the expression system; and (d) observing a suppression of the anion transport in the presence of the SLC26 activator and the test substance, whereby an inhibitor of SLC26 is determined. Optionally, the persistent activator and test substance can be provided to the functional expression simultaneously. Similarly, an assay for determining a SLC26 activator can comprise steps (a)-(d) above with the exception that an enhancement of conductance is observed in the presence of the persistent activator and the test substance.

Electrogenic Transport Assay. Anion transport via a SLC26 polypeptide of the present invention can further be determined to be electrogenic by monitoring changes in intracellular pH (pHᵢ₎and membrane voltage (Vₘ) during transport. Representative methods are described by Romero et al. (1998) Am J Physiol 274:F425-432 and Romero et al. (2000) J Biol Chem 275:24552-24559. See also Example 8.

Briefly, an oocyte is visualized with a dissecting microscope and held on a nylon mesh in a chamber having a volume of about 250 μl. The oocyte is continuously superfused with a saline solution (3 ml/minute to 5 ml/minute) that is delivered through TYGON® tubing (Worchester, Mass., United States of America). Solutions can be switched using a daisy-chain system of computer-actuated five-way valves with zero dead space. Solution changes in the chamber typically occur within 15 seconds to about 20 seconds. Membrane voltage (Vₘ) and intracellular pH (pHᵢ) of X. laevis oocytes are measured simultaneously using microelectrodes, as described by Romero et al. (1997) Nature 387:409-413.

Vₘ electrodes can be pulled from borosilicate fiber-capillary glass (Warner Instruments of West Haven, Conn., United States of America). Electrodes are backfilled with 3M KCl and typically have a resistance of about 3 MΩ) to 5 MΩ. The pH electrodes can be pulled in a similar manner, and are silanized by exposing them to 40 μl of bis-di-(methylamino)-dimethylsilane (Fluka Chemical of Ronkonkoma, N.Y., United States of America) for 5 minutes to 10 minutes. Silanized electrodes are deposited in an enclosed container at 2000C, and then baked overnight. The pH micropipettes are cooled under vacuum, and their tips are filled with hydrogen ionophore I-cocktail B (Fluka Chemical of Ronkonkoma, N.Y., United States of America). The pH micropipettes are then backfilled with a buffer containing 0.04M KH₂PO₄, 0.023M NaOH, and 0.015M NaCl (pH 7.0). Representative pH microelectrodes have slopes ranging from about −54 mV/pH unit to −59 mV/pH unit.

The Vₘ and pHᵢ electrodes are connected to high-impedance electrometers as described by Davis et al. (1992) Am J Physiol 263:C246-256 and Siebens & Boron (1989) Am J Physiol 256:F354-365. The voltage due to pH can be obtained by electronically subtracting the signals from the pH and Vₘ electrodes. Vₘ can be obtained by subtracting the signals from the Vₘ electrode and an external reference (calomel) electrode.

In accordance with the methods of the present invention, electrogenic transport can be detected using any suitable method. For example, pH can also be assayed by detecting the presence of a fluorescence dye, for example BCECF (available from Photon Technology International, Inc. of Lawrenceville, N.J., United States of America).

Vesicle Transport Assays. Once a SLC26 modulator has been identified, its effectiveness in modulating anion transport activity can further be tested in isolated membrane vesicles, including brush border membrane vesicles derived from kidney and gut. Modulators can also be tested for activity in cultured grafts, for example intact renal proximal tubules. Methods for preparing membrane vesicles and exografts are known in the art, and representative protocols are described by Pritchard & Miller (1993) Physiol Rev 73:765-796; Miller et al. (1996) Am J Physiol 271:F508-520; Masereeuw et al. (1996) Am J Physiol 271:F1173-1182; Masereeuw et al. (1999) J Pharmacol Exp Ther 289:1104-1111; Hagenbuch et al. (1985) Pflugers Arch 405:202-208; Kuo & Aronson (1988) J Biol Chem 263:9710-9717; and Pritchard & Renfro (1983) Proc Natl Acad Sci USA 80:2603-2607.

VII.E. Rational Design

The knowledge of the structure a native SLC26 polypeptide provides an approach for rational design of modulators and diagnostic agents. In brief, the structure of a SLC26 polypeptide can be determined by X-ray crystallography and/or by computational algorithms that generate three-dimensional representations. See Saqi et al. (1999) Bioinformatics 15:521-522; Huang et al. (2000) Pac Symp Biocomput:230-241; and PCT International Publication No. WO 99/26966. Alternatively, a working model of a SLC26 polypeptide structure can be derived by homology modeling (Maalouf et al., 1998). Computer models can further predict binding of a protein structure to various substrate molecules that can be synthesized and tested using the assays described herein above. Additional compound design techniques are described in U.S. Pat. Nos. 5,834,228 and 5,872,011.

In general, a SLC26 polypeptide is a membrane protein, and can be purified in soluble form using detergents or other suitable amphiphilic molecules. The resulting SLC26 polypeptide is in sufficient purity and concentration for crystallization. The purified SLC26 polypeptide preferably runs as a single band under reducing or non-reducing polyacrylamide gel electrophoresis (PAGE). The purified SLC26 polypeptide can be crystallized under varying conditions of at least one of the following: pH, buffer type, buffer concentration, salt type, polymer type, polymer concentration, other precipitating ligands, and concentration of purified SLC26. Methods for generating a crystalline polypeptide are known in the art and can be reasonably adapted for determination of a SLC26 polypeptide as disclosed herein. See e.g., Deisenhofer et al. (1984) J Mol Biol 180:385-398; Weiss et al. (1990) FEBS Lett 267:268-272; or the methods provided in a commercial kit, such as the CRYSTAL SCREEN™ kit (available from Hampton Research of Riverside, Calif., United States of America).

A crystallized SLC26 polypeptide can be tested for functional activity and differently sized and shaped crystals are further tested for suitability in X-ray diffraction. Generally, larger crystals provide better crystallography than smaller crystals, and thicker crystals provide better crystallography than thinner crystals. Preferably, SLC26 crystals range in size from 0.1-1.5 mm. These crystals diffract X-rays to at least 10 Å resolution, such as 1.5-10.0 Å or any range of value therein, such as 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 or 3, with 3.5 Å or less being preferred for the highest resolution.

VIII. Methods for Detecting a SLC26 Polypeptide

The present invention further provides methods for detecting a SLC26 polypeptide. The disclosed methods can be used for determining altered levels of SLC26 expression that are associated with SLC26A-related disorders and disease states.

In one embodiment of the invention, the method involves performing an immunochemical reaction with an antibody that specifically recognizes a SLC26 polypeptide, wherein the antibody was prepared according to a method of the present invention for producing such an antibody. Thus, the method comprises: (a) obtaining a biological sample comprising peptidic material; (b) contacting the biological sample with an antibody that specifically binds a SLC26 polypeptide and that was produced according to the disclosed methods, wherein the antibody comprises a detectable label; and (c) detecting the detectable label, whereby a SLC26 polypeptide in a sample is detected.

Techniques for detecting such antibody-antigen conjugates or complexes are known in the art and include but are not limited to centrifugation, affinity chromatography and other immunochemical methods. See e.g., Manson (1992) Immunochemical Protocols. Humana Press, Totowa, N.J., United States of America; Ishikawa (1999) Ultrasensitive and Rapid Enzyme Immunoassay. Elsevier, Amsterdam/New York, United States of America; Law (1996) Immunoassay: A Practical Guide. Taylor & Francis, London/Bristol, Pa., United States of America; Chan (1996) Immunoassay Automation: An Updated Guide to Systems. Academic Press, San Diego; Liddell & Weeks (1995) Antibody Technology. Bios Scientific Publishers, Oxford, United Kingdom; Masseyeff et al. (1993) Methods of Immunological Analysis. VCH Verlagsgesellschaft/VCH Publishers, Weinheim, Federal Republic of Germany/New York, United States of America; Walker & Rapley (1993) Molecular and Antibody Probes in Diagnosis. Wiley, Chichester, N.Y.; Wyckoff et al. (1985) Diffraction Methods for Biological Macromolecules. Academic Press, Orlando, Fla., United States of America; and references cited therein.

In another embodiment of the invention, a modulator that shows specific binding to a SLC26 polypeptide is used to detect a SLC26 anion transporter. Analogous to detection of a SLC26 polypeptide using an antibody, the method comprises: (a) obtaining a biological sample comprising peptidic material; (b) contacting the biological sample with a modulator of a SLC26 polypeptide, wherein the modulator comprises a detectable label; and (c) detecting the detectable label, whereby a SLC26 polypeptide in a sample is detected. Any suitable detectable label can be used, for example a fluorophore or epitope label.

IX. Therapeutic Applications

The present invention provides methods for identification of modulators of anion transport activity of SLC26A7 and SLC26A9. Alternatively, a construct encoding a recombinant SLC26A7 or SLC26A9 polypeptide can be used to replace diminished or lost SLC26 function. The modulators and constructs of the invention are useful for regulation of anion transport in a subject, for example to remedy dysfunctional anion transport associated with sulphate homeostasis, sulphation, oxalate homeostasis, transepithelial salt transport, bicarbonate transport, and physiological pH regulation.

The term “subject” as used herein includes any vertebrate species, preferably warm-blooded vertebrates such as mammals and birds. More particularly, the methods of the present invention are contemplated for the treatment of tumors in mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants and livestock (such as cattle, oxen, sheep, giraffes, deer, goats, bison, and camels), and horses. Also contemplated is the treatment of birds, including those kinds of birds that are endangered or kept in zoos, as well as fowl, and more particularly domesticated fowl or poultry, such as turkeys, chickens, ducks, geese, guinea fowl, and the like, as they are also of economical importance to humans.

SLC26A7-Related Disorders. Functional characterization of SLC26A7, as disclosed herein, indicates that it can mediate Cl⁻—Cl⁻ exchange. The chloride transport properties of SLC26A7 point to its role as a swelling-activated chloride transporter. Northern blot analysis revealed renal SLC26A7 expression, particularly in renal papilla (Example 5). Swelling-activated efflux of chloride and other osmolytes has been reported in a number of cells, including those of the inner medullary collecting duct (Boese et al., 1996), which presumably express SLC26A7. In hepatocytes, chloride transport is required for cell volume regulation and acidification of intracellular organelles. In response to cell swelling, hepatocyts increase their chloride conductance by as much as 30-fold to 100-fold (Jackson et al., 1996; Meng & Weinman, 1996). Both the magnitude of this effect and its physiological role are particularly significant in hepatocytes. Unlike most other cell types, hepatocyte function requires cycles of swelling and shrinkage. Hepatocytes take up large quantities of amino acids and other nutrients and consequently swell in response to meals (Wang & Wondergem, 1993). The ability of hepatocytes to regulate volume depends on a rapid increase in chloride conductance.

The molecular identity of the swelling activated osmolyte/anion channel or transporter is still unclear (Kirk & Strange, 1998; Strange, 1998). However, recent studies suggest that anion exchangers are involved (Sanchez-Olea et al., 1995; Fievet et al., 1998). In this regard, SLC26A7 and/or other SLC26 exchangers could function in volume-sensitive osmolyte transport in the kidney and other tissues.

SLC26A9-Related Disorders. The regulation airway surface liquid is an important part of lung defense mechanisms and can contribute to airway disease (Noone et al., 1994). Recent studies also suggest that the composition of airway surface liquid is regulated by active ion transport systems (Tarran et al., 2001).

In particular, Cl⁻—HCO₃₋ exchange via SLC26A9, as disclosed herein, is potentially relevant to the HCO₃ excretion that is directed by CFTR, a chloride channel whose dysfunction results in cystic fibrosis. The Cl⁻ base exchange properties of SLC26A9 suggest that it may encode the apical CFTR-dependent bicarbonate transporter in lung (Lee et al., 1998). A major controversy in cystic fibrosis research is whether CFTR itself encodes a bicarbonate-permeable channel, or whether it regulates the activity of a separate bicarbonate transporter/channel (Ulrich, 2000). The fact that bicarbonate transport mediated by SLC26A9 is electrogenic, as disclosed herein, suggests that it could encode a CFTR-dependent electrogenic bicarbonate pathway in pulmonary epithelial cells.

Studies of cystic fibrosis pancreatic cell lines have shown that expression of wild type CFTR can elicit elevated levels of Cl⁻—HCO₃ exchange (Elgavish & Meezan, 1992; Greeley et al., 2001). In addition, the inability of CFTR mutants to regulate Cl⁻—HCO₃⁻ exchange is correlated with the pancreatic insufficiency (Choi et al., 2001). The presence of a type I PDZ interaction motif at the extreme C-terminus of SLC26A9 suggests that it can collaborate with CFTR and other transporters at the apical membrane of pulmonary epithelial cells (Wang et al., 2000; Ahn et al., 2001).

SLC26A9 shows lung-specific expression, including expression in the Calu-3 pulmonary cell line, a model for pulmonary submucosal gland serous epithelial cells (Lee et al., 1998). Submucosal gland serous epithelial cells are a particular prominent site of CFTR expression, and are thought to play a major role in pulmonary fluid, Cl⁻, and HCO₃⁻ bicarbonate excretion (Ballard et al., 1999).

The present invention provides that SLC26A9 can mediate Cl⁻-base (Cl⁻—OH and/or Cl⁻—HCO₃⁻) exchange and is expressed in cells relevant to CF disease. Thus, modulators of SLC26A9 can be used to activate Cl⁻—HCO₃⁻-exchange in CF patients.

SLC26A9 is also a positional candidate for pseudohypoaldosteronism type II (Gordon's syndrome), an autosomal hypertensive disorder with linkage to this region of chromosome 1 (Mansfield, 1997). PHA-II is characterized by hyperkalaemia despite normal renal glomerular filtration, hypertension and correction of physiologic abnormalities by thiazide diuretics. Mild hyperchloremia, metabolic acidosis, and suppressed plasma renin activity can also be manifest. Clinical studies of PHAII pathogenesis indicate an abnormality in renal ion transport and have invoked a so-called “chloride-shunt”, whereby chloride transport in the distal nephron is selectively upregulated. Analysis of linkage in eight PHAII families showing autosomal dominant transmission demonstrates locus heterogeneity of this trait, with a multilocus lod score of 8.1 for linkage of PHAII to chromosomes 1q31-q42, a region that includes SLC26A9. SLC26A9 transcripts can be amplified by RT-PCR from human kidney RNA, which is consistent with a role for SLC26A9 in regulating renal salt excretion.

X. Therapeutic Compositions and Methods

In accordance with the methods of the present invention, a composition that is administered to alter anion transport activity in a subject comprises: (a) an effective amount of a SLC26 modulator; and (b) a pharmaceutically acceptable carrier. A SLC26 modulator can comprise any one of the types of test substances described herein above. A SLC26 modulator can also comprise a pH modifier.

The present invention also provides methods for modulating anion transport activity in a subject via administration of a gene therapy construct comprising an SLC26 polypeptide. Such a construct can be prepared as described herein above, further comprising a carrier suitable for administration to a subject.

X.A. pH Modifiers

In one embodiment of the invention, a method is provided for modulating SLC26 anion transport by administering a modulator of a SLC26 polypeptide to the subject, wherein the modulator comprises a pH modifier.

The term “pH modifier” refers to any substance that can be used to regulate the pH of an in situ environment. An effective amount of a pH modifier comprises an amount sufficient to alter a pH to a level sufficient for activation of a SLC26 polypeptide. An effective amount of a pH modifier effective to achieve the desired in vivo pH modification will depend on the acidity or basicity (pKa or pKb) of the compound used, the pH of the carrier (e.g., a polymer composition) used when in vivo, and the in vivo environment's physiologic pH.

Representative pH modifiers include acidic compounds or anhydrous precursors thereof, or chemically protected acids. For example, a pH modifier can comprise at least one member selected from the group consisting of: amino acids; carboxylic acids and salts thereof; di-acids and salts thereof; poly-acids and salts thereof; esters that are easily hydrolyzable in vivo; lactones that are easily hydrolyzable in vivo; organic carbonates; enolic compounds; acidic phenols; polyphenolic compounds; aromatic alcohols; ammonium compounds or salts thereof; boron-containing compounds; sulfonic acids and salts thereof; sulfinic acids and salts thereof; phosphorus-containing compounds; acid halides; chloroformates; acid gases; acid anhydrides; inorganic acids and salts thereof; and polymers having functional groups of at least one of the preceding members. A pH modifier of this invention can also comprise at least one member selected from the group consisting of: glycine; alanine; proline; lysine; glutaric acid; D-galacturonic acid; succinic acid; lactic acid; glycolic acid; poly(acrylic acid); sodium acetate; diglycolic anhydride; succinic anhydride; citraconic anhydride; maleic anhydride; lactide; diethyl oxalate; Meldrum's acid; diethyl carbonate; dipropyl carbonate; diethyl pyrocarbonate; diallyl pyrocarbonate; di-tert-butyl dicarbonate; ascorbic acid; catechin; ammonium chloride; D-glucosamine hydrochloride; 4-hydroxy-ephedrine hydrochloride; boric acid; nitric acid; hydrochloric acid; sulfuric acid; ethanesulfonic acid; and p-toluenesulfonic acid; 2-aminoethylphosphoric acid; methylphosphonic acid; dimethylphosphinic acid; methyl chloroformate; sulfur dioxide; and carbon dioxide.

A pH modifier can be prepared in a microcapsule, such that the pH modifier diffuses through the microcapsule or is released by bioerosion of the microcapsule. The microcapsule may be formulated so that the pH modifier is released from the microcapsule continuously over a period of time. Microencapsulation of the pH modifier can be achieved by many known microencapsulation techniques, as described further herein below under the heading “Carriers.”

X.B. Carriers

Any suitable carrier that facilitates preparation and/or administration of a SLC26 modulator can be used. The carrier can be a viral vector or a non-viral vector. Suitable viral vectors include adenoviruses, adeno-associated viruses (AAVs), retroviruses, pseudotyped retroviruses, herpes viruses, vaccinia viruses, Semiliki forest virus, and baculoviruses.

Suitable non-viral vectors that can be used to deliver a SLC26 polypeptide or a SLC26 modulator include but are not limited to a plasmid, a nanosphere (Manome et al., 1994; Saltzman & Fung, 1997), a peptide (U.S. Pat. Nos. 6,127,339 and 5,574,172), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al., 1997) and U.S. Pat. Nos. 4,551,482, 5,714,166, 5,510,103, 5,490,840, and 5,855,900), and a polysome (U.S. Pat. No. 5,922,545).

Where appropriate, two or more types of carriers can be used together. For example, a plasmid vector can be used in conjunction with liposomes.

A carrier can be selected to effect sustained bioavailability of a SLC26 modulator to a site in need of treatment. The term “sustained bioavailability” encompasses factors including but not limited to prolonged release of a SLC26 modulator from a carrier, metabolic stability of a SLC26 modulator, systemic transport of a composition comprising a SLC26 modulator, and effective dose of a SLC26 modulator.

Representative compositions for sustained bioavailability can include but are not limited to polymer matrices, including swelling and biodegradable polymer matrices, (U.S. Pat. Nos. 6,335,035; 6,312,713; 6,296,842; 6,287,587; 6,267,981; 6,262,127; and 6,221,958), polymer-coated microparticles (U.S. Pat. Nos. 6,120,787 and 6,090,925) a polyol:oil suspension (U.S. Pat. No. 6,245,740), porous particles (U.S. Pat. No. 6,238,705), latex/wax coated granules (U.S. Pat. No. 6,238,704), chitosan microcapsules, and microsphere emulsions (U.S. Pat. No. 6,190,700).

Microcapsules. Microencapsulation can be carried out by dissolving a coating polymer in a volatile solvent, e.g., methylene chloride, to a polymer concentration of about 6% by weight; adding a pH modifying compound (selected to be acidic or basic according to the pH level to be achieved in situ) in particulate form to the coating polymer/solvent solution under agitation, to yield a pH modifier concentration of 2% to 10% by weight; adding the resulting polymer dispersion to a methylene chloride solution containing a phase inducer, such as silicone oil, under agitation; allowing the mixture to equilibrate for about 20 minutes; further adding the mixture slowly to a non-solvent, such as heptane, under rapid agitation; allowing the more volatile solvent to evaporate under agitation; removing the agitator; separating the solids from the silicone oil and heptane; and washing and drying the microcapsules. The size of the microcapsules will range from about 0.001 to about 1000 microns. See e.g., U.S. Pat. No. 6,061,581.

A microencapsulating coating polymer is preferably biodegradable and/or can permit diffusion of the encapsulated modulator (e.g., a pH modifier). A microencapsulating coating also preferably has low inherent moisture content. Biodegradation preferably occurs at rates greater than or similar to the rate of degradation of the base polymer.

Examples of coating materials that can be used to microencapsulate a SLC26 modulator, for example a pH modifier, include but are not limited to polyesters, such as polyglycolic acid, polylactic acid, copolymers of polyglycolic acid and polylactic acid, polycaprolactone, poly-β-hydroxybutyrate, copolymers of ε-caprolactone and δ-valerolactone, copolymers of α-caprolactone and DL-dilactide, and polyester hydrogels; polyvinylpyrrolidone; polyamides; gelatin; albumin; proteins; collagen; poly(orthoesters); poly(anhydrides); poly(alkyl-2-cyanoacrylates); poly(dihydropyrans); poly(acetals); poly(phosphazenes); poly(urethanes); poly(dioxinones); cellulose; and starches.

Viral Gene Therapy Vectors. Viral vectors of the invention are preferably disabled, e.g. replication-deficient. That is, they lack one or more functional genes required for their replication, which prevents their uncontrolled replication in vivo and avoids undesirable side effects of viral infection. Preferably, all of the viral genome is removed except for the minimum genomic elements required to package the viral genome incorporating the therapeutic gene into the viral coat or capsid. For example, it is desirable to delete all the viral genome except: (a) the Long Terminal Repeats (LTRs) or Invented Terminal Repeats (ITRs); and (b) a packaging signal. In the case of adenoviruses, deletions are typically made in the E1 region and optionally in one or more of the E2, E3 and/or E4 regions. Other viral vectors can be similarly deleted of genes required for replication. Deletion of sequences can be achieved by a recombinant approach, for example, involving digestion with appropriate restriction enzymes, followed by re-ligation. Replication-competent self-limiting or self-destructing viral vectors can also be used.

Nucleic acid constructs of the invention can be incorporated into viral genomes by any suitable approach known in the art. Typically, such incorporation is performed by ligating the construct into an appropriate restriction site in the genome of the virus. Viral genomes can then be packaged into viral coats or capsids using any suitable procedure. In particular, any suitable packaging cell line can be used to generate viral vectors of the invention. These packaging lines complement the replication-deficient viral genomes of the invention, as they include, for example by incorporation into their genomes, the genes that have been deleted from the replication-deficient genome. Thus, the use of packaging lines allows viral vectors of the invention to be generated in culture.

Suitable packaging lines for retroviruses include derivatives of PA317 cells, ψ-2 cells, CRE cells, CRIP cells, E-86-GP cells, and 293GP cells. Line 293 cells are preferred for use with adenoviruses and adeno-associated viruses.

Plasmid Gene Therapy Vectors. A SLC26 modulator or SLC26 polypeptide can also be encoded by a plasmid. Advantages of a plasmid carrier include low toxicity and easy large-scale production. A polymer-coated plasmid can be delivered using electroporation as described by Fewell et al. (2001) Mol Ther 3:574-583. Alternatively, a plasmid can be combined with an additional carrier, for example a cationic polyamine, a dendrimer, or a lipid, that facilitates delivery. See e.g., Baher et al. (1999) Anticancer Res 19:2917-2924; Maruyama-Tabata et al. (2000) Gene Ther 7:53-60; and Tam et al. (2000) Gene Ther 7:1867-1874.

Liposomes. A composition of the invention can also be delivered using a liposome. Liposomes can be prepared by any of a variety of techniques that are known in the art. See e.g.,—(1997). Current Protocols in Human Genetics on CD-ROM. John Wiley & Sons, New York; Lasic & Martin (1995) STEALTH® Liposomes. CRC Press, Boca Raton, Fla., United States of America; Janoff (1999) Liposomes: Rational Design. M. Dekker, New York; Gregoriadis (1993) Liposome Technology, 2nd ed. CRC Press, Boca Raton, Fla., United States of America; Betageri et al. (1993) Liposome Drug Delivery Systems. Technomic Pub., Lancaster; Pa., United States of America.; and U.S. Pat. Nos. 4,235,871; 4,551,482; 6,197,333; and 6,132,766. Temperature-sensitive liposomes can also be used, for example THERMOSOMES™ as disclosed in U.S. Pat. No. No. 6,200,598. Entrapment of a SLC26 modulator or a SLC26 polypeptide within liposomes of the present invention can be carried out using any conventional method in the art. In preparing liposome compositions, stabilizers such as antioxidants and other additives can be used.

Other lipid carriers can also be used in accordance with the claimed invention, such as lipid microparticles, micelles, lipid suspensions, and lipid emulsions. See e.g., Labat-Moleur et al. (1996) Gene Therapy 3:1010-1017; and U.S. Pat. Nos. 5,011,634; 6,056,938; 6,217,886; 5,948,767; and 6,210,707.

X.B. Targeting Ligands

As desired, a composition of the invention can include one or more ligands having affinity for a specific cellular marker to thereby enhance delivery of a SLC26 modulator or a SLC26 polypeptide to a site in need of treatment in a subject. Ligands include antibodies, cell surface markers, peptides, and the like, which act to home the therapeutic composition to particular cells.

The terms “targeting” and “homing”, as used herein to describe the in vivo activity of a ligand following administration to a subject, each refer to the preferential movement and/or accumulation of a ligand in a target tissue (e.g., a tumor) as compared with a control tissue.

The term “target tissue” as used herein refers to an intended site for accumulation of a ligand following administration to a subject. For example, the methods of the present invention employ a target tissue comprising a tumor. The term “control tissue” as used herein refers to a site suspected to substantially lack binding and/or accumulation of an administered ligand.

The terms “selective targeting” of “selective homing” as used herein each refer to a preferential localization of a ligand that results in an amount of ligand in a target tissue that is about 2-fold greater than an amount of ligand in a control tissue, more preferably an amount that is about 5-fold or greater, and most preferably an amount that is about 10-fold or greater. The terms “selective targeting” and “selective homing” also refer to binding or accumulation of a ligand in a target tissue concomitant with an absence of targeting to a control tissue, preferably the absence of targeting to all control tissues.

The terms “targeting ligand” and “targeting molecule” as used herein each refer to a ligand that displays targeting activity. Preferably, a targeting ligand displays selective targeting. Representative targeting ligands include peptides and antibodies.

The term “peptide” encompasses any of a variety of forms of peptide derivatives, that include amides, conjugates with proteins, cyclized peptides, polymerized peptides, conservatively substituted variants, analogs, fragments, peptoids, chemically modified peptides, and peptide mimetics. Representative peptide ligands that show tumor-binding activity include, for example, those described in U.S. Pat. Nos. 6,180,084 and 6,296,832.

The term “antibody” indicates an immunoglobulin protein, or functional portion thereof, including a polyclonal antibody, a monoclonal antibody, a chimeric antibody, a hybrid antibody, a single chain antibody (e.g., a single chain antibody represented in a phage library), a mutagenized antibody, a humanized antibody, and antibody fragments that comprise an antigen binding site (e.g., Fab and Fv antibody fragments). See U.S. Pat. Nos. 5,111,867; 5,632,991; 5,849,877; 5,948,647; 6,054,561 and PCT International Publication No. WO 98/10795.

Antibodies, peptides, or other ligands can be coupled to drugs (e.g., a SLC26 modulator or a gene therapy construct comprising a SLC26 polypeptide) or drug carriers using methods known in the art, including but not limited to carbodiimide conjugation, esterification, sodium periodate oxidation followed by reductive alkylation, and glutaraldehyde crosslinking. See e.g., Bauminger & Wilchek (1980) Methods Enzymol 70:151-159;

Goldman et al. (1997) Cancer Res 57:1447-1451; Kirpotin et al. (1997) Biochemistry 36:66-75; —(1997). Current Protocols in Human Genetics on CD-ROM. John Wiley & Sons, New York; Neri et al. (1997) Nat Biotechnol 15:1271-1275; Park et al. (1997) Cancer Lett 118:153-160; and Pasqualini et al. (1997) Nat Biotechnol 15:542-546; U.S. Pat. No. 6,071,890; and European Patent No. 0 439 095. Alternatively, pseudotyping of a retrovirus can be used to target a virus towards a particular cell (Marin et al., 1997).

X.C. Formulation

Suitable formulations for administration of a composition of the invention to a subject include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some preferred ingredients are sodium dodecyl sulphate (SDS), for example in the range of 0.1 to 10 mg/ml, preferably about 2.0 mg/ml; and/or mannitol or another sugar, for example in the range of 10 to 100 mg/ml, preferably about 30 mg/ml; phosphate-buffered saline (PBS), and any other formulation agents conventional in the art.

The therapeutic regimens and compositions of the invention can be used with additional adjuvants or biological response modifiers including, but not limited to, the cytokines interferon alpha (IFN-α), interferon gamma (IFN-γ), interleukin 2 (IL2), interleukin 4 (IL4), interleukin 6 (IL6), tumor necrosis factor (TNF), or other cytokine affecting immune cells.

X.D. Dose and Administration

A composition of the present invention can be administered to a subject systemically, parenterally, or orally. The term “parenteral” as used herein includes intravenous injection, intra-muscular injection, intra-arterial injection, and infusion techniques. For delivery of compositions to pulmonary pathways, compositions can be administered as an aerosol or coarse spray. A delivery method is selected based on considerations such as the type of the type of carrier or vector, therapeutic efficacy of the composition, and the condition to be treated.

Preferably, an effective amount of a composition of the invention is administered to a subject. For example, an “effective amount” is an amount of a composition sufficient to modulate SLC26 anion transport activity.

Actual dosage levels of active ingredients in a therapeutic composition of the invention can be varied so as to administer an amount of the composition that is effective to achieve the desired therapeutic response for a particular subject. The selected dosage level will depend upon a variety of factors including the activity of the therapeutic composition, formulation, the route of administration, combination with other drugs or treatments, the disease or disorder to be treated, and the physical condition and prior medical history of the subject being treated. Determination and adjustment of an effective amount or dose, as well as evaluation of when and how to make such adjustments, are known to those of ordinary skill in the art of medicine.

For local administration of viral vectors, previous clinical studies have demonstrated that up to 10¹³ pfu (plaque forming units) of virus can be injected with minimal toxicity. In human patients, 1×10⁹-1×10¹³ pfu are routinely used. See Habib et al. (1999) Hum Gene Ther 10:2019-2034. To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose is also modified to maximize the desired modulation of anion transporter activity.

For soluble formulations of a composition of the present invention, conventional methods of extrapolating human dosage are based on doses administered to a murine animal model can be carried out using the conversion factor for converting the mouse dosage to human dosage: Dose Human per kg=Dose Mouse per kg×12 (Freireich et al., 1966). Drug doses are also given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al. (1966) Cancer Chemother Rep 50:219-244. Briefly, to express a mg/kg dose in any given species as the equivalent mg/m² dose, the dose is multiplied by the appropriate km factor. In adult humans, 100 mg/kg is equivalent to 100 mg/kg×37 kg/m²=3700 mg/m².

For additional guidance regarding dose, see Berkow et al. (1997) The Merck Manual of Medical Information, Home ed. Merck Research Laboratories, Whitehouse Station, N.J., United States of America; Goodman et al. (1996) Goodman & Gilman's the Pharmacological Basis of Therapeutics, 9th ed. McGraw-Hill Health Professions Division, New York; Ebadi (1998) CRC Desk Reference of Clinical Pharmacology. CRC Press, Boca Raton, Fla., United States of America; Katzung (2001) Basic & Clinical Pharmacology, 8th ed. Lange Medical Books/McGraw-Hill Medical Pub. Division, New York; Remington et al. (1975) Remington's Pharmaceutical Sciences, 15th ed. Mack Pub. Co., Easton, Pa.; Speight et al. (1997) Avery's Drug Treatment: A Guide to the Properties. Choice, Therapeutic Use and Economic Value of Drugs in Disease Management, 4th ed. Adis International, Auckland/Philadelphia, United States of America; Duch et al. (1998) Toxicol Lett 100-101:255-263.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Cloning of Mouse and Human SLC26A7

Human SLC26A7 exons were initially identified in draft sequences of the BAC clone RP11-353D5 (Genbank AC017061) by performing tBLASTn searches of the HTGS database (available at http://www.ncbi.nim.nih.gov/HTGSi/) using SLC26A 1, SLC26A2, SLC26A3, and SLC26A4 as query sequences. A BLASTn search of human ESTs using the extracted exon contig yielded two human ESTs. The corresponding I.M.A.G.E. clones were obtained from Research Genetics, Inc. (Huntsville, Ala., United States of America) and sequenced on both strands using fluorescent dye terminator chemistry (available from Applied Biosystems of Foster City, Calif., United States of America). The RP11-353D5 contig did not contain 5′ exons from the SLC26A7 gene. The 5′ exons were identified using GENSCAN (Burge & Karlin, 1997; Burge & Karlin, 1998; available at http://bioweb.pasteur.fr/seqanal/interfaces/genscan.html) on a 500,000 base pair contig containing the entire gene (Celera of Rockville, Md., United States of America).

The 5′ end of human SLC26A7cDNA was then cloned from reverse transcribed human kidney RNA (Clontech of Palo Alto, Calif., United States of America) using a sense primer in coding exon 1 (SEQ ID NO:22) and an anti-sense primer within coding exon 8 (SEQ ID NO:23). PCR conditions were optimized using Taq2000 and the OptiPrime buffer system (Stratagene of La Jolla, Calif., United States of America) as described Mount et al. (1999) J Biol Chem 274:16355-16362. Amplified PCR fragments were subcloned into the pCR2.1 vector (Invitrogen Corporation of Carlsbad, Calif.).

A BLASTn search of mouse ESTs using human SLC26A7cDNA as a query sequence yielded a full-length mouse SLC26A7 EST clone (I.M.A.G.E. clone 4020760), which was sequenced in entirety. The Mouse Genome Database (MGD, available at http://www.informatics.jax.org/mgihome/) was used as a resource for the analysis of murine genomic contigs.

Analysis of nucleotide and amino acid sequences was performed using VECTOR NTI® 6.0 software (InforMax, Inc. of Bethesda, Md., United States of America), GRAIL® software (Lockheed Martin Energy Research Corporation of Oak Ridge, Tenn., United States of America) (Roberts, 1991; Xu et al., 1994; Uberbacher et al., 1996; available at http://compbio.ornl.gov/Grail-1.3), Phosphobase (Kreegipuu et al., 1999; available at http://www.cbs.dtu.dk/databases/PhosphoBase/), TESS (Schug & Overton, 1997; available at http://www.cbil.upenn.edu/cqi-bin/tess), Matinspector (Quandt et al., 1995; available from Genomatix Software GMBH of Munich Germany and at http://transfac.gbf.de/cgi-bin/amtSearch/matsearch.pl), Prosite (Bucher & Bairoch, 1994; Hofmann et al., 1999; available at http://www.expasy.ch/prosite/), Scansite (Songyang et al., 1993; Songyang et al., 1997; Yaffe et al., 1997; available at (http://cansite.bidmc.harvard.edu/cantley85.html) and PFAM (Bateman et al., 2000; available at http://www.sanger.ac.uk/Software/Pfam/).

The predicted mouse and human SLC26A7 proteins are both 656 amino acids in length, and exhibit 88% identity. The SLC26A7 sequences encode two domains associated with the SLC26 gene family, a central “sulphate transporter family” domain (PFAM Domain No. PF00916) between residues 162 to 472 (FIG. 1) and a C-terminal “sulphate transporter and anti-sigma factor” domain (STAS domain, PFAM Domain No. PF01740) (Aravind & Koonin, 2000) between residues 493-637. The central core is also moderately homologous to the xanthine/uracil permease domain (PFAM Domain No. PF00860), with an E value of 0.037 (<0.05 is significant). The SLC26A7 proteins also contain a predicted type I PDZ interaction motif (Songyang et al., 1997), S-E-V near the C-terminus of each protein.

### Example 2

### Cloning of Mouse and Human SLC26A9

Human SLC26A9 exons were initially identified in draft sequences of BAC clones RP11-196022 and RP11-37015. Whereas the first coding exon was identified in these contigs using GENSCAN (Burge & Karlin, 1997; Burge & Karlin, 1998; available at http://bioweb.pasteur.fr/seqanal/interfaces/genscan.html), neither gene annotation programs nor homology to other SLC26 proteins were useful for identification of the last coding exon. Therefore, 3′ genomic contigs were used to search human ESTs for potential 3′-UTR ESTs. One such EST cDNA, I.M.A.G.E. clone 2915384, was sequenced in its entirety. This cDNA includes nucleotides 2502 to 4526 of the final human SLC26A9 cDNA sequence.

The open reading frame was then cloned from human lung, using LA TAQ™ DNA polymerase (TaKaRa of Verivers, Belgium) and primers (SEQ ID NOs:24-25) according to the following conditions: 30 cycles of denaturation at 98° C. for 30 seconds and amplification/extension at 68° C. for 6 minutes. Amplified PCR products were subcloned into the pCR2.1 vector (Invitrogen Corporation of Carlsbad, Calif., United States of America) and sequenced.

To identify mouse SLC26A9, a BLASTn search of mouse genomic sequences (Celera of Rockville, Md., United States of America) was performed using human SLC26A9 cDNA as a query sequence. A 500,000 base pair contig containing the entire mouse SLC26A9 gene was identified. The Mouse Genome Database (MGD, available at http://www.informatics.jax.org/mgihome/) was used as a resource for the analysis of murine genomic contigs. A 3049 base pair cDNA encompassing the entire mouse SLC26A9 open reading frame was then amplified by RT-PCR from mouse lung, using LA TAQ™ DNA polymerase (TaKaRa of Verivers, Belgium) and primers (SEQ ID NOs:26-27) as described above for human SLC26A9.

Analysis of nucleotide and amino acid sequences was performed as described in Example 1. The human SLC26A9 transcript is alternatively spliced such that splicing of a short intron not conserved in the mouse gene leads to a protein that is longer by 96 amino acids. Human SLC26A9 transcripts in which this intron is retained predict a protein of 791 amino acids, essentially the same length as the mouse protein. Mouse SLC26A9 and the shorter human SLC26A9 isoform both end in a predicted C-terminal type I PDZ interaction motif (S-E-V).

### Example 3

### Genomic Localization of SLC26A7

The mouse and human SLC26A7 genes each span ˜130 kb of genomic DNA, and share an identical organization with 19 coding exons. Genomic contigs containing the human SLC26A7 gene contain a number of mapped markers. These include the marker D8S1476 (Genbank G08719) and the STS WI-16423 (Genbank G21154), each of which had been localized to chromosome 8q22.2. Genomic localization of human STS markers was performed using the LDB (Location Database, http://cedar.genetics.soton.ac.uk/public html).

The mouse SLC26A7 gene is in the syntenic region of chromosome 4 at about 4 cM, based on the localization of the physically linked genes MMP-16, calbindin-1, and CBFA2T1 (Niwa-Kawakita et al., 1995).

### Example 4

### Genomic Localization of SLC26A9

Genomic contigs encompassing human SLC26A9 did not contain mapped genes or STS markers, and thus SLC26A9 was mapped by radiation hybrid analysis. The Stanford G3 panel (Research Genetics, Inc. of Huntsville, Ala., United States of America) was screened by PCR using a primer pair from within the 3′-UTR of SLC26A9 (SEQ ID NOs:28-29). Amplification products were alkali-denatured, applied to a nylon membrane using a dot-blot apparatus, and subjected to Southern blotting with a ³²P-labeled internal oligonucleotide probe (SEQ ID NO:28). Results were analyzed by querying the Stanford RH map (http://www-shgc.stanford.edu/RH/).

The SLC26A9 gene was found to be tightly linked to D1S456 on chromosome 1q32, between markers D1S306 and D1S491. Mouse SLC26A9 was determined to be physically linked to several STS markers and to the cathepsin E, Mdm4, ELK4, and PCTK3 genes, all of which had been localized on the syntenic region of murine chromosome 1 at about 70 cM (MGD).

### Example 5

### Northern Blot Anlaysis of SLC26A7 and SLC26A9 Expression

RNA was extracted from C57BU6J mice and human cell lines using guanidine isothiocyanate and cesium chloride. The Panc-1 and Calu-3 cell lines were obtained from the American Type Culture Collection (ATCC of Manassas, Va., United States of America) and grown in DMEM with 10% FBS. Calu-3 is a model for pulmonary submucosal gland serous epithelial cells (Lee et al., 1998), and Panc-1 is a model for pancreatic ductal epithelial cells (Elgavish & Meezan, 1992). Total RNA (10 ag/lane) was size-fractionated by electrophoresis (5% formaldehyde, 1% agarose), and transferred to a nylon membrane (Stratagene of La Jolla, Calif., United States of America). Amplifed fragments of mouse SLC26A7, mouse SLC26A9, and GAPDH were labeled with ³²P by the random priming method (DecaPrime kit for randomly-prime labeling, available from Ambion, Inc. of Austin, Tex., United States of America). The blots were sequentially hybridized and stripped according to standard methods known in the art.

Northern blots prepared using 2 μg/lane of human poly-A⁺ RNA were purchased from Clontech of Palo Alto, Calif., United States of America, and were hybridized to SLC26-specific probes and to a human β-actin probe.

Hybridization of all blots was performed overnight at 42° C. in 4×SSCP/40% formamide/4× Denhart's solution/0.5% SDS/200 μg salmon sperm DNA. Membranes were washed twice for 10 minutes at room temperature in 2×SSCP/0.1% SDS, and twice for 1 hour at 65° C. in 0.1×SSCP/0.1% SDS.

Mouse SLC26A7 transcripts were observed in a restricted expression pattern, with a 6 kb transcript detected only in testis, renal outer medulla, and renal papilla. Human SLC26A7 transcripts were not detected using commercial Northern blots.

A 5 kb SLC26A9 transcript was detected specifically in mouse and human lung. Thus, human SLC26A9 is co-expressed with SLC26A6 in the Calu-3 cell line, a well-defined model for pulmonary submucosal gland serous epithelial cells (Lee et al., 1998).

### Example 6

### Expression of SLC26A7 and SLC26A9 in Xenopus laevis Oocytes

Full-length mouse SLC26A7 and SLC26A9 cDNAs were cloned into the Xenopus expression vector pGEMHE (Liman et al., 1992). SLC26A6 and SLC26A2 constructs were also prepared for use as controls in transport assays (Example 7). Expression constructs were linearized, and cRNA was transcribed in vitro using T7 RNA polymerase and a MMESSAGE MMACHINE® transcription kit (Ambion, Inc. of Austin, Tex., United States of America). Defolliculated oocytes were injected with 25 nl to 50 nl of water or with a solution containing cRNA at a concentration of 0.5 μg/μl (12.5 ng to 25 ng per oocyte) using a Nanoliter-2000 injector (WPI Instruments of Sarasota, Fla., United States of America). Oocytes were incubated at 17° C. in 50% Leibovitz's L-15 media supplemented with penicillin/streptomycin (1000 units/ml) and glutamine for 2-3 days for uptake assays.

### Example 7

### Anion Transport Assays

For sulphate uptake assays, oocytes were pre-incubated for 20 minutes in chloride-free uptake medium (100 mM NMDG gluconate, 2 mM potassium gluconate, 1 mM calcium gluconate, 1 mM magnesium gluconate, 10 mM HEPES-Tris, pH 6.0 or pH 7.5 as indicated), followed by a 60-minute period for uptake in the same medium supplemented with 1 mM K₂³⁵SO₄ (40 μCi/ml). The cells were then washed three times in uptake buffer with 5 mM cold K₂SO₄ to remove tracer activity in the extracellular fluid. The oocytes were dissolved individually in 10% SDS, and tracer activity was determined by scintillation counting. Uptake of chloride, formate, and oxalate was assayed using the same chloride-free uptake solutions, substituting 8.3 mM ³⁶Cl, 500 μM [¹⁴C]oxalate, or 50 μM [¹⁴C]formate for labeled sulphate. Each uptake experiment included 12-18 oocytes in each experimental group, and results were reported as means±SEM.

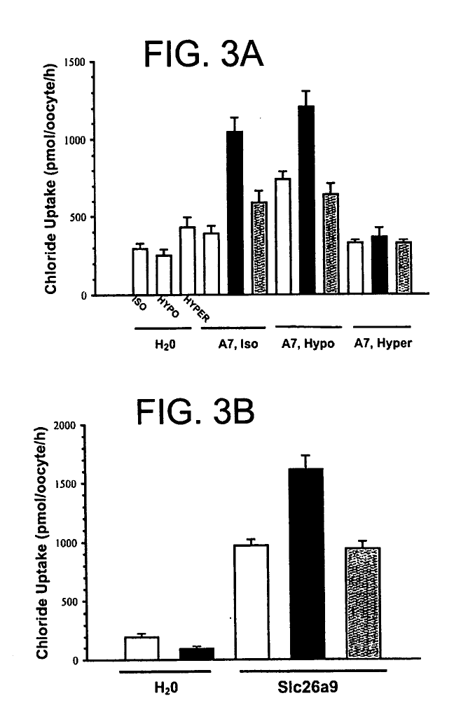

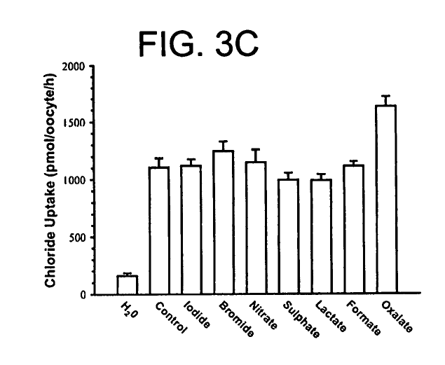

For sulphate exchange and cis-inhibition experiments, the concentration of NMDG-gluconate in the uptake solution was adjusted to maintain isotonic osmolality (−210 mOsm/Kg), which was confirmed experimentally using a FISKE® osmometer (Fiske Associates, Inc. of Bethel, Conn., United States of America). For the experiments presented in FIG. 3A, the concentration of NMDG-gluconate medium osmolality was adjusted downwards to generate hypotonic conditions (120 mOsm/Kg), whereas mannitol was added to generate hypertonic conditions (300 mOsm/Kg).

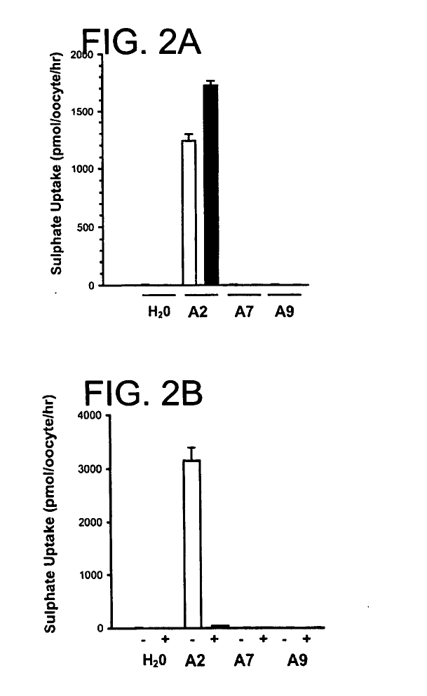

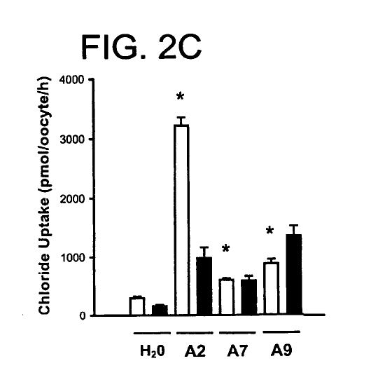

FIG. 2 shows the results of ³⁵SO₄²⁻ and ³⁶Cl⁻ uptake studies for SLC26A7 and SLC26A9. Uptakes were performed at pH 7.4 and pH 6.0, to study the effect of an acid-outside pH gradient. ³⁵SO₄²⁻ uptake assays were also performed in the presence and absence of extracellular Cl⁻, which activates SLC26A1 (Satoh et al., 1998). SLC26A2-injected oocytes mediated robust ³⁵SO₄²⁻ uptake which is increased significantly at pH 6.0 (FIG. 2A). SLC267 and SLC26A9 did not appear to mediate significant ³⁵SO₄²⁻ uptake in comparison to water-injected controls. The addition of extracellular Cl⁻ significantly inhibited ³⁵SO₄²⁻ uptake by SLC26A2 without activating the uptake in SLC26A7 and SLC26A9-injected oocytes.

SLC26A2-injected oocytes also mediated significant ³⁶Cl⁻ uptake, which is inhibited by an acid-outside pH gradient, at extracellular pH of 6.0 (3220±136 at pH 7.4, versus 981±181 pmol/oocyte/hr at pH 6.0) (FIG. 2C). Since the concentration of Cl⁻ in Xenopus oocyte cytoplasm is −30 mM (Romero et al., 2000), versus 8 mM in the extracellular uptake medium, a significant component of the uptake activity probably represents Cl⁻—Cl⁻ exchange.

SLC26A7-injected oocytes demonstrated modest ³⁶Cl⁻ uptake activity which was equivalent at pH 7.4 and 6.0 (605±29 and 596±76 pmol/oocyte/hr, respectively), and which was significantly higher (p<0.00006) than that of water-injected cells (301±33 and 166±20 pmol/oocyte/h) (FIG. 2C). The Cl⁻—Cl⁻ exchange mediated by SLC26A9 was very reproducible, but the absolute values of ³⁶Cl⁻ uptakes for this transporter were much lower than those for SLC26A2, SLC26A6, and SLC26A9.

There was also a modest increase in SLC26A7 ³⁶Cl⁻ uptake activity under hypotonic conditions (cell swelling), but the activity of the SLC26A7-injected oocytes was no different from water-injected controls under hypertonic conditions (cell shrinkage) (FIG. 3A).

SLC26A9-injected oocytes mediated ³⁶Cl⁻ uptakes of 876 i 76 and 1339±169 pmol/oocyte/hr at pH 7.4 and pH 6.0, respectively (FIG. 2C). The modest difference between SLC26A9 uptakes at pH 7.4 and 6.0 was not statistically significant when data were analyzed from four separate experiments with a combined total of −60 oocytes per group (1648±200 and 1971±192 pmol/oocyte/hr at pH 7.4 and 6.0, respectively, p<0.24).

SLC26A7 activity was modestly DIDS-sensitive at pH 6.0, under both isotonic and hypotonic conditions (FIG. 3A), similar to that observed for SLC26A1, SLC26A2, and SLC26A6 activity. Cl⁻—Cl⁻ exchange mediated by SLC26A9 was also moderately sensitive to DIDS (FIG. 3B).

A functional characteristic common to all of the SLC26 anion exchangers is cis-inhibition of the uptake of a given ion by other transported substrates (Satoh et al., 1998; Moseley et al., 1999b; Scott & Kamiski, 2000b; Knauf et al., 2001). ³⁶Cl⁻ uptake mediated by SLC26A9 in the presence of several monovalent and divalent anions was not inhibited by any of the substrates tested (FIG. 3C).

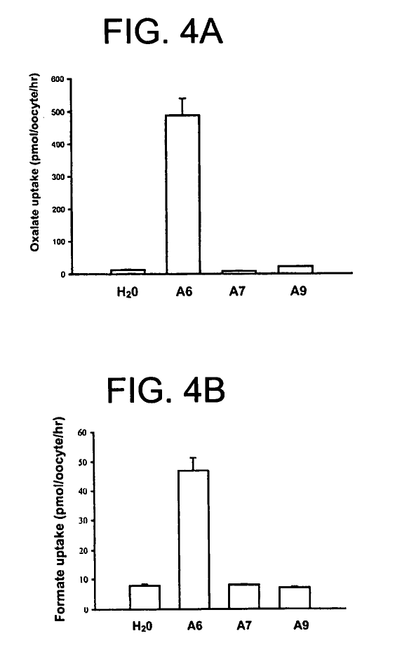

Neither SLC26A7 nor SLC26A9 mediated oxalate or formate uptake in Xenopus oocytes (FIGS. 4A and 4B, respectively), which is consistent with the cis-inhibition results for SLC26A9. SLC26A6-injected oocytes were used served as a positive control for oxalate and formate transport.

### Example 8

### Electrogenic Transport Assays

For electrophysiological measurements, oocytes were studied 3 days to 11 days following injection of SLC26 expression constructs. CO₂/HCO₃-free ND96 medium contained 96 mM NaCl, 2 mM KCl, 1 mM MgCl₂, 1.8 mM CaCl₂, and 5 mM HEPES (pH 7.5 and 195-200mOsm). For CO₂/HCO₃-equilibrated solutions, 33 mM NaHCO₃ replaced 33 mM NaCl. In “0-Na+” solutions, choline replaced Na⁺. In “0-Cl” solutions, gluconate replaced Cl⁻. All solutions were titrated to pH 7.5, and were continuously bubbled with CO₂-balanced O₂ to maintain pCO₂ and pH. Ion selective microelectrodes were prepared, calibrated, and employed as described by Romero et al. (1998) Am J Physiol 274:F425-432 and by Romero et al. (2000) J Biol Chem 275:24552-24559. All pH electrodes had slopes of at least −56 mV/decade change.

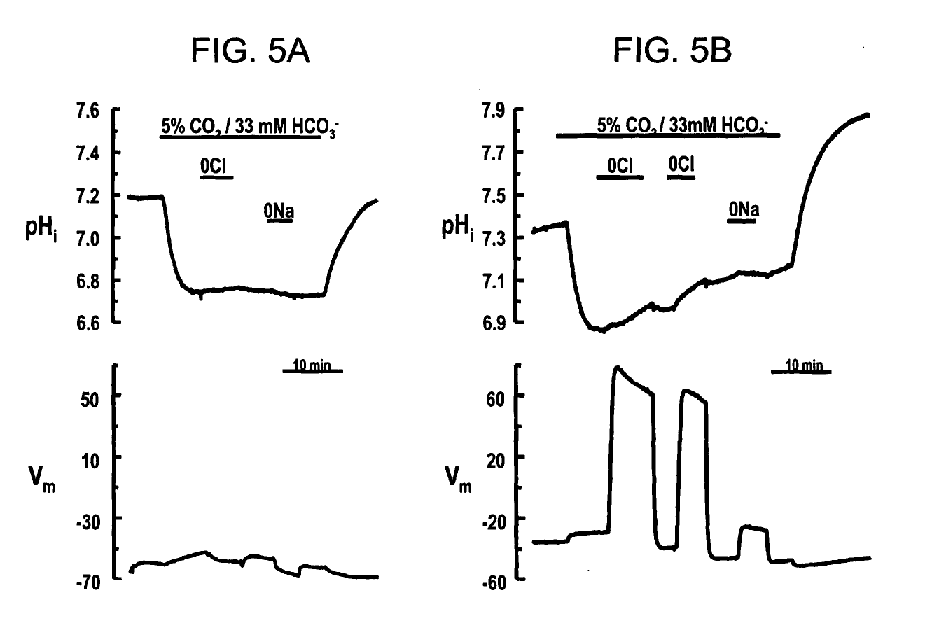

To determine whether SLC26A7 and SLC26A (function as Cl⁻—HCO₃⁻ exchangers, intracellular pH (pHᵢ) was measured in response to the manipulation of bath HCO₃⁻ and Cl⁻. The initial addition of CO₂/HCO₃ to the bath solution resulted in the acidification of oocytes due to CO₂ plasma membrane diffusion, then intracellular hydration and dissociation forming intracellular H⁺ and HCO₃′. FIG. 5A shows that a water-injected oocyte exposed to 5% CO2/33 mM HCO₃⁻ (pH 7.5) acidified by 0.44 pH units at an initial rate of 46×10⁴ pH units/sec (460×10⁵ pH units/sec). The initial intracellular pH (pHᵢ) of SLC26A9-injected oocytes is essentially the same as that of water controls, and addition of 5% CO₂/33 mM HCO₃ produced a fall in pHᵢ of 0.50 pH units at an initial rate of 350×10⁻⁵ pH units/sec in the experiment shown (average of −496±40×10⁻⁵ pH units/sec, n=8). The addition of HCO₃⁻ produced a slight but abrupt depolarization in Slc26a9-injected oocytes (10.0±0.8 mV, n=8). Replacement of Cl⁻ (gluconate) did not change pHᵢ or Vₘ observed in the water control. In contrast, FIG. 5B shows that Cl⁻ removal increased pHᵢ in SLC26A9-injected oocytes at a rate of 44×10⁻⁵ pH units/sec (average of +55±16×10⁻⁵ pH units/sec, n=8), which ceased after Cl⁻ re-addition. Surprisingly, gluconate replacement evoked a 105 mV depolarization (95.9±6.3 mV, n=8). A second Cl⁻ removal increased the alkalinization rate to 28×10⁻⁵ pH units/sec (+23±9.4×10⁻⁵ pH units/sec, n=6) and reproduced the depolarization (96.0±7.8 mV, n=6).

To test the cation depdence of SLC26A9, Na⁺ was replaced with choline. Na⁺ removal reduced pHᵢ (−23±7.6×0-5 pH units/sec, n=4) and depolarized the oocytes (26.8±1.6 mV, n=8) (FIG. 5B). Prior to CO₂ removal, pHᵢ rose to 7.2, which is approximately the non-HCO₃⁻ level. Removal of 5% CO₂/33 mM HCO₃⁻ elicited a robust alkalinization and pHᵢ overshoot to 7.9 (7.79±0.11, n=7), which is indicative of cellular HCO₃-loading (+0.37±0.06 pH units above starting pHᵢ) (Romero, 1998; Romero, 2000). This overshoot was not observed in controls. SLC26A9-injected oocytes also showed an abrupt hyperpolarization (˜5-7 mV) with removal of CO₂/HCO₃⁻.

FIG. 5B illustrates the results of an experiment using individual water-injected and SLC26A9-injected oocytes. These observations have been repeated using SLC26A9-injected oocytes from five separate Xenopus and averaged as noted.

### Example 9

### Generation and Characterization of SLC26A9/Slc26a9- and SLC26A7/Slc26a7-Specific Antibodies

The availability of the entire mammalian SLC26 gene family enables the design and generation of paralog-specific reagents, including antibodies. Polyclonal rabbit anti-peptide antibodies were generated against an N-terminal peptide from the Slc26a7 protein and against two C-terminal peptides from the Slc26a9 protein. The N-terminal Slc26a7 peptide chosen corresponds to residues 8-25 of the predicted protein (KRSVLWGKMHTPHREDIK—SEQ ID NO:31). The C-terminal Slc26a9 peptides correspond to residues 596-612 (QELQQDFESAPSTDPNN—SEQ ID NO:32) and 565-584 (KQKYLRKQEKRTAIPTQQRK—SEQ ID NO:33). In all three cases the peptide epitopes are free from sequence identity with other members of the SLC26 gene family and are predicted to be highly antigenic.

Rabbits were immunized against these peptides, followed by ELISA testing of reactivity against the peptide antigens; in all cases high titre (≧1:10,000) antisera have been obtained. Affinity purification of polyclonal antibody from late-immunization bleeds and Western blotting was performed as described by Mount, D. B. et al., Am J Physiol 276:F347-F358 (1999).

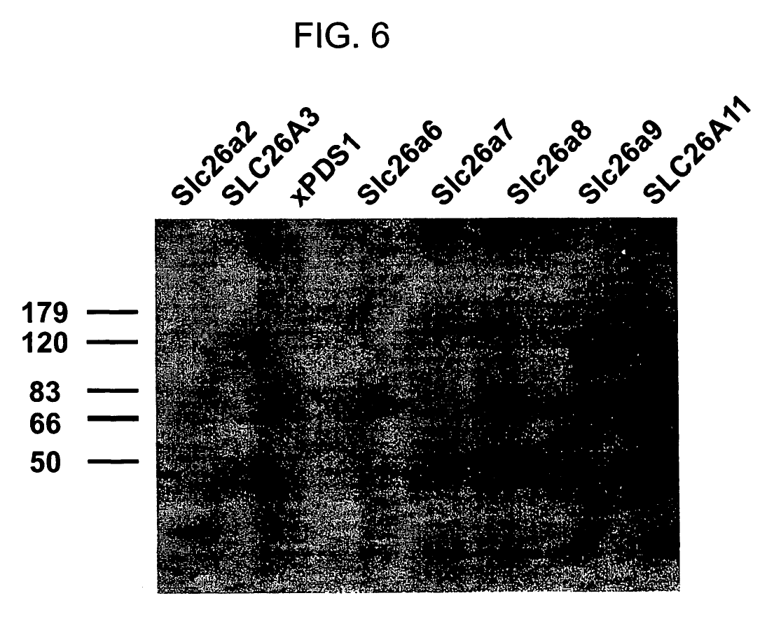

FIG. 6 shows a Western blot of lysate samples from Xenopus oocytes expressing several SLC26 paralogs, using a 1:300 titre of the C-terminal Slc26a9 antibody generated against the 596-612 peptide. The Slc26a9 anti-sera clearly recognizes Slc26a9 protein with a molecular mass of −120 kDa, corresponding to glycosylated Slc26a9 protein; no other Slc26/SLC26 proteins are recognized by the antibody, proving that it is a highly specific reagent for the mouse Slc26a9 paralog. Since the antigenic peptide only differs from the human SLC26A9 protein at 2 residues, this antisera is also predicted to react with the human ortholog. Knowledge of the entire mouse and human Slc26/SLC26 gene family thus played a role in the design and generation of paralog-specific reagents. In the case of SLC26A9, precise tissue and membrane localization in lung and kidney facilitate understanding of its role in both cystic fibrosis and autosomal-dominant hypertension with hyperkalemia on chromosome 1 (Mansfield, T. A. et al., 1997), respectively.

The SLC26A7/Slc26a7 is particularly abundant within the renal papilla, however the cell type wherein it is expressed (epithelial, vascular, or interstitial) and cell membrane domain (apical vs. basolateral) are not yet known. A polyclonal rabbit antibody was generated against peptides 8-25 of the murine Slc26a7 protein (KRSVLWGKMHTPHREDIK—SEQ ID NO:31). Affinity purification of polyclonal antibody from late-immunization bleeds and Western blotting was performed as described by Mount, D. B. et al., Am J Physiol 276:F347-F358 (1999).

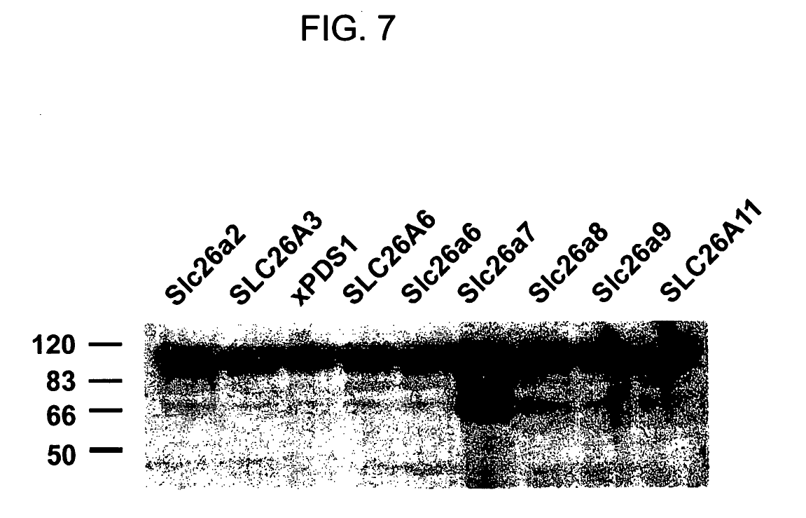

FIG. 7 shows a Western blot of lysate samples from Xenopus oocytes expressing several SLC26 paralogs, using a 1:300 titre of the C-terminal Slc26a7 antibody. Although a 120 kDa protein is detected in all lanes, likely due to a non-specific interaction, a 70 kDa core protein and a 90 kDa complex glycoprotein are clearly seen only in the Slc26a7 lane. Presumably these non-specific bands will disappear in mammalian tissue and/or at a lower titre, using re-purified antibody. Again, the provision of the Sc26a7-specific antibody will facilitate the understanding of its physiological role in kidney and elsewhere.

### REFERENCES

The references listed below as well as all references cited in the specification are incorporated herein by reference to the extent that they supplement, explain, provide a background for or teach methodology, techniques and/or compositions employed herein.
(1997) Current Protocols in Human Genetics on Cd-Rom. In. New York: John Wiley & Sons.
Ahn W, Kim K H, Lee J A, Kim J Y, Choi J Y, Moe O W, Milgram S L, Muallem S & Lee M G (2001) Regulatory Interaction between the Cystic Fibrosis Transmembrane Conductance Regulator and HCO₃⁻ Salvage Mechanisms in Model Systems and the Mouse Pancreatic Duct. J Biol Chem 276:17236-17243.
Altschul S F, Gish W, Miller W, Myers E W & Lipman D J (1990) Basic Local Alignment Search Tool. J Mol Biol 215:403-410.
Aravind L & Koonin E V (2000) The STAS Domain—a Link between Anion Transporters and Antisigma—Factor Antagonists. Curr Biol 10:R53-55.
Ausubel F, ed (1995) Short Protocols in Molecular Biology, 3rd ed. Wiley, New York.
Baher A G, Andres M L, Folz-Holbeck J, Cao J D & Gridley D S (1999) A Model Using Radiation and Plasmid-Mediated Tumor Necrosis Factor-Alpha Gene Therapy for Treatment of Glioblastomas. Anticancer Res 19:2917-2924.
Ballard S T, Trout L, Bebok Z, Sorscher E J & Crews A (1999) CFTR Involvement in Chloride, Bicarbonate, and Liquid Secretion by Airway Submucosal Glands. Am J Physiol 277:L694-699.
Barton G J (1998) Protein Sequence Alignment Techniques. Acta Crystallogr D Biol Crystallogr 54:1139-1146.
Bateman A, Birney E, Durbin R, Eddy S R, Howe K L & Sonnhammer E L (2000) The PFAM Protein Families Database. Nucleic Acids Res 28:263-266.
Batzer M A, Carlton J E & Deininger P L (1991) Enhanced Evolutionary PCR Using Oligonucleotides with Inosine at the 3′-Terminus. Nucleic Acids Res 19:5081.
Bauminger S & Wilchek M (1980) The Use of Carbodiimides in the Preparation of Immunizing Conjugates. Methods Enzymol 70:151-159.
Berkow R, Beers M H & Fletcher A J (1997) The Merck Manual of Medical Information, Home ed. Merck Research Laboratories, Whitehouse Station, N.J., United States of America.
Betageri G V, Jenkins S A & Parsons D L (1993) Liposome Drug Delivery Systems. Technomic Pub., Lancaster, Pa., United States of America.
Bissig M, Hagenbuch B, Stieger B, Koller T & Meier P J (1994) Functional Expression Cloning of the Canalicular Sulfate Transport System of Rat Hepatocytes. J Biol Chem 269:3017-3021.
Biwersi J & Verkman A S (1991) Cell-Permeable Fluorescent Indicator for Cytosolic Chloride. Biochemistry 30:7879-7883.
Biwersi J, Tulk B & Verkman A S (1994) Long-Wavelength Chloride-Sensitive Fluorescent Indicators. Anal Biochem 219:139-143.
Bodanszky M (1993) Principles of Peptide Synthesis, 2nd rev. ed. Springer-Verlag, Berlin/New York.
Boese S H, Wehner F & Kinne R K (1996) Taurine Permeation through Swelling-Activated Anion Conductance in Rat IMCD Cells in Primary Culture. Am J Physiol 271:F498-507.
Brookes A J (1999) The Essence of Snps. Gene 234:177-186.
Bucher P & Bairoch A (1994) A Generalized Profile Syntax for Biomolecular Sequence Motifs and Its Function in Automatic Sequence Interpretation. Proc Int Conf Intell Syst Mol Biol 2:53-61.
Burge C & Karlin S (1997) Prediction of Complete Gene Structures in Human Genomic DNA. J Mol Biol 268:78-94.
Burge C B & Karlin S (1998) Finding the Genes in Genomic DNA. Curr Opin Struct Biol 8:346-354.
Capecchi M R (1989a) Altering the Genome by Homologous Recombination. Science 244:1288-1292.
Capecchi M R (1989b) Altering the Genome by Homologous Recombination. Science 244:1288-1292.
Chan D W (1996) Immunoassay Automation: An Updated Guide to Systems. Academic Press, San Diego, Calif., United States of America.
Chernova M N, Jiang L, Crest M, Hand M, Vandorpe D H, Strange K & Alper S L (1997) Electrogenic Sulfate/Chloride Exchange in Xenopus Oocytes Mediated by Murine AE1 E699q. J Gen Physiol 109:345-360.
Chiang L W (1998) Detection of Gene Expression in Single Neurons by Patch-Clamp and Single-Cell Reverse Transcriptase Polymerase Chain Reaction. J Chromatogr A 806:209-218.
Conner B J, Reyes A A, Morin C, Itakura K, Teplitz R L & Wallace R B (1983) Detection of Sickle Cell Beta S-Globin Allele by Hybridization with Synthetic Oligonucleotides. Proc Natl Acad Sci USA 80:278-282.
Davis B A, Hogan E M & Boron W F (1992) Activation of Na-H Exchange by Intracellular Lithium in Barnacle Muscle Fibers. Am J Physiol 263:C246-256.
Deisenhofer J, Epp O, Miki K, Huber R & Michel H (1984) X-Ray Structure Analysis of a Membrane Protein Complex. Electron Density Map at 3 a Resolution and a Model of the Chromophores of the Photosynthetic Reaction Center from Rhodopseudomonas Viridis. J Mol Biol 180:385-398.
Delpire E, Lu J, England R, Dull C & Thorne T (1999) Deafness and Imbalance Associated with Inactivation of the Secretory Na-K-2CI Co-Transporter. Nat Genet 22:192-195.
Duch D S, Rehberg B & Vysotskaya T N (1998) Volatile Anesthetics Significantly Suppress Central and Peripheral Mammalian Sodium Channels. Toxicol Lett 100-101:255-263.
Ebadi M S (1998) CRC Desk Reference of Clinical Pharmacology. CRC Press, Boca Raton, Fla., United States of America.
Elgavish A & Meezan E (1992) Altered Sulfate Transport Via Anion Exchange in CFPAC Is Corrected by Retrovirus-Mediated CFTR Gene Transfer. Am J Physiol 263:C176-186.
European Patent No. 0 439 095
Everett L A & Green E D (1999) A Family of Mammalian Anion Transporters and Their Involvement in Human Genetic Diseases. Hum Mol Genet 8:1883-1891.
Fewell J G, MacLaughlin F, Mehta V, Gondo M, Nicol F, Wilson E & Smith L C (2001) Gene Therapy for the Treatment of Hemophilia B Using Pinc-Formulated Plasmid Delivered to Muscle with Electroporation. Mol Ther 3:574-583.
Fievet B, Perset F, Gabillat N, Guizouarn H, Borgese F, Ripoche P & Motais R (1998) Transport of Uncharged Organic Solutes in Xenopus Oocytes Expressing Red Cell Anion Exchangers (AE1s). Proc Natl Acad Sci USA 95:10996-11001.
Freireich E J, Gehan E A, Rail D P, Schmidt L H & Skipper H E (1966) Quantitative Comparison of Toxicity of Anticancer Agents in Mouse, Rat, Hamster, Dog, Monkey, and Man. Cancer Chemother Rep 50:219-244.
Freshney R1 (1987) Culture of Animal Cells: A Manual of Basic Technique, 2nd ed. A.R. Liss, New York.
Galietta L J, Springsteel M F, Eda M, Niedzinski E J, By K, Haddadin M J, Kurth M J, Nantz M H & Verkman A S (2001) Novel CFTR Chloride Channel Activators Identified by Screening of Combinatorial Libraries Based on Flavone and Benzoquinolizinium Lead Compounds. J Biol Chem 276:19723-19728.
Glover D M & Hames B D (1995) DNA Cloning: A Practical Approach, 2nd ed. IRL Press at Oxford University Press, Oxford/New York.
Goldman C K, Rogers B E, Douglas J T, Sosnowski B A, Ying W, Siegal G P, Baird A, Campain J A & Curiel D T (1997) Targeted Gene Delivery to Kaposi's Sarcoma Cells Via the Fibroblast Growth Factor Receptor. Cancer Res 57:1447-1451.
Goodman L S, Gilman A, Hardman J G, Gilman A G & Limbird L E (1996) Goodman & Gilman's the Pharmacological Basis of Therapeutics, 9th/ed. McGraw-Hill Health Professions Division, New York.
Greeley T, Shumaker H, Wang Z, Schweinfest C W & Soleimani M (2001) Downregulated in Adenoma and Putative Anion Transporter Are Regulated by CFTR in Cultured Pancreatic Duct Cells. Am J Physiol Gastrointest Liver Physiol 281:G1301-1308.
Gregoriadis G (1993) Liposome Technology, 2nd ed. CRC Press, Boca Raton, Fla., United States of America.
Habib N A, Hodgson H J, Lemoine N & Pignatelli M (1999) A Phase I/II Study of Hepatic Artery Infusion with WT53-CMV-Ad in Metastatic Malignant Liver Tumours. Hum Gene Ther 10:2019-2034.
Hagenbuch B, Stange G & Murer H (1985) Transport of Sulphate in Rat Jejunal and Rat Proximal Tubular Basolateral Membrane Vesicles. Pflugers Arch 405:202-208.
Harlow E & Lane D (1988) Antibodies: A Laboratory Manual. Cold Spring Harbor Laboratory Press, Cold Spring Harbor, N.Y.
Henikoff J G, Pietrokovski S, McCallum C M & Henikoff S (2000) Blocks-Based Methods for Detecting Protein Homology. Electrophoresis 21:1700-1706.
Henikoff S & Henikoff J G (1992) Amino Acid Substitution Matrices from Protein Blocks. Proc Natl Acad Sci USA 89:10915-10919.
Henikoff S & Henikoff J G (2000) Amino Acid Substitution Matrices. Adv Protein Chem 54:73-97.
Hofmann K, Bucher P, Falquet L & Bairoch A (1999) The Prosite Database, Its Status in 1999. Nucleic Acids Res 27:215-219.
Homola J, Yee S & Gauglitz G (1999) Surface Plasmone Resnoance Sensors: Review. Sensors and Actuators B 54:3-15.
Huang C C, Novak W R, Babbitt P C, Jewett A I, Ferrin T E & Klein T E (2000) Integrated Tools for Structural and Sequence Alignment and Analysis. Pac Symp Biocomput:230-241.
Hutchens & Yip (1993) Rapid Communications in Mass Spectroscopy 7:576-580.
International Publication No. WO 98/10795
International Publication No. WO 99/26966
Ishikawa E (1999) Ultrasensitive and Rapid Enzyme Immunoassay. Elsevier, Amsterdam/New York.
Jackson P S, Churchwell K, Ballatori N, Boyer J L & Strange K (1996) Swelling-Activated Anion Conductance in Skate Hepatocytes:

Regulation by Cell Cl⁻ and ATP. Am J Physiol 270:C57-66.
Jayaraman S, Teitler L, Skalski B & Verkman A S (1999) Long-Wavelength Iodide-Sensitive Fluorescent Indicators for Measurement of Functional CFTR Expression in Cells. Am J Physiol 277:C1008-1018.
Jayaraman S, Haggie P, Wachter R M, Remington S J & Verkman A S (2000) Mechanism and Cellular Applications of a Green Fluorescent Protein-Based Halide Sensor. J Biol Chem 275:6047-6050.
Joyner A L (1993) Gene Targeting: A Practical Approach. Oxford University Press, Oxford/New York.
Karlin S & Altschul S F (1993) Applications and Statistics for Multiple High-Scoring Segments in Molecular Sequences. Proc Natl Acad Sci USA 90:5873-5877.
Karniski L P (1989) Activation of Cl/OH Exchange by Parachloromercuribenzoic Acid in Rabbit Renal Brush-Border Membranes. J Membr Biol 112:59-66.
Karniski L P, Lotscher M, Fucentese M, Hilfiker H, Biber J & Murer H (1998) Immunolocalization of Sat-1 Sulfate/Oxalate/Bicarbonate Anion Exchanger in the Rat Kidney. Am J Physiol 275:F79-87.
Katzung B G (2001) Basic & Clinical Pharmacology, 8th ed. Lange Medical Books/McGraw-Hill Medical Pub. Division, New York.
Kere J, Lohi H & Hoglund P (1999) Genetic Disorders of Membrane Transport Iii. Congenital Chloride Diarrhea. Am J Physiol 276:G7-G13.
Kestila M, Lenkkeri U, Mannikko M, Lamerdin J, McCready P, Putaala H, Ruotsalainen V, Morita T, Nissinen M, Herva R, Kashtan C E, Peltonen L, Holmberg C, Olsen A & Tryggvason K (1998) Positionally Cloned Gene for a Novel Glomerular Protein—Nephrin—ls Mutated in Congenital Nephrotic Syndrome. Mol Cell 1:575-582.
Khurana O K, Coupland L A, Shelden M C & Howitt S M (2000) Homologous Mutations in Two Diverse Sulphate Transporters Have Similar Effects. FEBS Lett 477:118-122.
Kirk K & Strange K (1998) Functional Properties and Physiological Roles of Organic Solute Channels. Annu Rev Physiol 60:719-739.
Kirpotin D, Park J W, Hong K, Zalipsky S, Li W L, Carter P, Benz C C & Papahadjopoulos D (1997) Sterically Stabilized Anti-Her2 Immunoliposomes: Design and Targeting to Human Breast Cancer Cells in Vitro. Biochemistry 36:66-75.
Knauf F, Yang C L, Thomson R B, Mentone S A, Giebisch G & Aronson P S (2001) Identification of a Chloride-Formate Exchanger Expressed on the Brush Border Membrane of Renal Proximal Tubule Cells. Proc Natl Acad Sci USA 98:9425-9430.
Kreegipuu A, Blom N & Brunak S (1999) Phosphobase, a Database of Phosphorylation Sites: Release 2.0. Nucleic Acids Res 27:237-239.
Krick W, Dolle A, Hagos Y & Burckhardt G (1998) Characterization of the Chloride Conductance in Porcine Renal Brush-Border Membrane Vesicles. Pflugers Arch 435:415-421.
Kuo S M & Aronson P S (1988) Oxalate Transport Via the Sulfate/Hco3 Exchanger in Rabbit Renal Basolateral Membrane Vesicles. J Biol Chem 263:9710-9717.
Kyte J & Doolittle R F (1982) A Simple Method for Displaying the Hydropathic Character of a Protein. J Mol Biol 157:105-132.
Labat-Moleur F, Steffan A M, Brisson C, Perron H, Feugeas O, Furstenberger P, Oberling F, Brambilla E & Behr J P (1996) An Electron Microscopy Study into the Mechanism of Gene Transfer with Lipopolyamines. Gene Ther 3:1010-1017.
Landegren U, Nilsson M & Kwok P Y (1998) Reading Bits of Genetic Information: Methods for Single-Nucleotide Polymorphism Analysis. Genome Res 8:769-776.
Lasic D D & Martin F J (1995) Stealth® Liposomes. CRC Press, Boca Raton, Fla., United States of America.
Lee M C, Penland C M, Widdicombe J H & Wine J J (1998) Evidence That Calu-3 Human Airway Cells Secrete Bicarbonate. Am J Physiol 274:L450-453.
Liddell E & Weeks I (1995) Antibody Technology. Bios Scientific Publishers, Oxford, United Kingdom.
Liman E R, Tytgat J & Hess P (1992) Subunit Stoichiometry of a Mammalian K⁺ Channel Determined by Construction of Multimeric cDNAs. Neuron 9:861-871.
Maalouf G J, Xu W, Smith T F & Mohr S C (1998) Homology Model for the Ligand-Binding Domain of the Human Estrogen Receptor. J Biomol Struct Dyn 15:841-851.
Mak P, McDonnell D P, Weigel N L, Schrader W T & O'Malley B W (1989) Expression of Functional Chicken Oviduct Progesterone Receptors in Yeast (Saccharomyces Cerevisiae). J Biol Chem 264:21613-21618.
Malmquist (1993) Nature 361:186-187.
Manome Y, Abe M, Hagen M F, Fine H A & Kufe D W (1994) Enhancer Sequences of the Df3 Gene Regulate Expression of the Herpes Simplex Virus Thymidine Kinase Gene and Confer Sensitivity of Human Breast Cancer Cells to Ganciclovir. Cancer Res 54:5408-5413.
Mansfield T A, Simon D B, Farfel Z, Bia M, Tucci J R, Lebel M, Gutkin M, Vialettes B, Christofilis M A, Kauppinen-Makelin R, Mayan H, Risch N & Lifton R P (1997) Multilocus Linkage of Familial Hyperkalaemia and Hypertension, Pseudohypoaldosteronism Type II, to Chromosomes 1q31-42 and 17p11-Q21. Nature Genetics 16:202-205.
Manson M M (1992) Immunochemical Protocols. Humana Press, Totowa, N.J., United States of America.
Mansoura M K, Biwersi J, Ashlock M A & Verkman A S (1999) Fluorescent Chloride Indicators to Assess the Efficacy of CFTR cDNA Delivery. Hum Gene Ther 10:861-875.
Marin M, Noel D & Piechaczyk M (1997) Towards Efficient Cell Targeting by Recombinant Retroviruses. Mol Med Today 3:396-403.
Maruyama-Tabata H, Harada Y, Matsumura T, Satoh E, Cui F, Iwai M, Kita M, Hibi S, Imanishi J, Sawada T & Mazda O (2000) Effective Suicide Gene Therapy in Vivo by EBV-Based Plasmid Vector Coupled with Polyamidoamine Dendrimer. Gene Ther 7:53-60.
Masereeuw R, Russel F G & Miller D S (1996) Multiple Pathways of Organic Anion Secretion in Renal Proximal Tubule Revealed by Confocal Microscopy. Am J Physiol 271:F1173-1182.
Masereeuw R, Moons M M, Toomey B H, Russel F G & Miller D S (1999) Active Lucifer Yellow Secretion in Renal Proximal Tubule: Evidence for Organic Anion Transport System Crossover. J Pharmacol Exp Ther 289:1104-1111.
Masseyeff R F, Albert W H W & Staines N (1993) Methods of Immunological Analysis. VCH Verlagsgesellschaft; VCH Publishers, Weinheim (Federal Republic of Germany)/New York, N.Y. (United States of America).
Melvin J E, Park K, Richardson L, Schultheis P J & Shull G E (1999) Mouse Down-Regulated in Adenoma (DRA) Is an Intestinal Cl(−)/HCO₃ Exchanger and Is up-Regulated in Colon of Mice Lacking the NHE3 Na(+)/H(+) Exchanger. J Biol Chem 274:22855-22861.
Meng X J & Weinman S A (1996) cAMP-and Swelling-Activated Chloride Conductance in Rat Hepatocytes. Am J Physiol 271:C112-120.
Miller T M & Johnson E M, Jr. (1996) Metabolic and Genetic Analyses of Apoptosis in Potassium/Serum-Deprived Rat Cerebellar Granule Cells. J Neurosci 16:7487-7495.
Moseley R H, Hoglund P, Wu G D, Silberg D G, Haila S, de la Chapelle A, Holmberg C & Kere J (1999) Downregulated in Adenoma Gene Encodes a Chloride Transporter Defective in Congenital Chloride Diarrhea. Am J Physiol 276:G185-192.
Mount D B, Mercado A, Song L, Xu J, George A L, Jr., Delpire E & Gamba G (1999) Cloning and Characterization of KCC3 and KCC4, New Members of the Cation-Chloride Cotransporter Gene Family. J Biol Chem 274:16355-16362.
Mount, D. B. et al. Isoforms of the apical Na-K-2Cl transporter in murine thick ascending limb. I: Molecular characterization and intra-renal localization. Am J Physiol 276:F347-F358 (1999).
Needleman S B & Wunsch C D (1970) A General Method Applicable to the Search for Similarities in the Amino Acid Sequence of Two Proteins. J Mol Biol 48:443-453.
Neri D, Carnemolla B, Nissim A, Leprini A, Querze G, Balza E, Pini A, Tarli L, Halin C, Neri P, Zardi L & Winter G (1997) Targeting by Affinity-Matured Recombinant Antibody Fragments of an Angiogenesis Associated Fibronectin Isoform. Nat Biotechnol 15:1271-1275.
Nickerson D A, Kaiser R, Lappin S, Stewart J, Hood L & Landegren U (1990) Automated DNA Diagnostics Using an ELISA-Based Oligonucleotide Ligation Assay. Proc Natl Acad Sci USA 87:8923-8927.
Noone P G, Olivier K N & Knowles M R (1994) Modulation of the Ionic Milieu of the Airway in Health and Disease. Annu Rev Med 45:421-434.
Ohtsuka E, Matsuki S, Ikehara M, Takahashi Y & Matsubara K (1985) An Alternative Approach to Deoxyoligonucleotides as Hybridization Probes by Insertion of Deoxyinosine at Ambiguous Codon Positions. J Biol Chem 260:2605-2608.
Orita M, Iwahana H, Kanazawa H, Hayashi K & Sekiya T (1989) Detection of Polymorphisms of Human DNA by Gel Electrophoresis as Single-Strand Conformation Polymorphisms. Proc Natl Acad Sci USA 86:2766-2770.
Park J W, Hong K, Kirpotin D B, Meyer 0, Papahadjopoulos D & Benz C C (1997) Anti-Her2 Immunoliposomes for Targeted Therapy of Human Tumors. Cancer Lett 118:153-160.
Pasqualini R, Koivunen E & Ruoslahti E (1997) Alpha V Integrins as Receptors for Tumor Targeting by Circulating Ligands. Nat Biotechnol 15:542-546.
Quandt K, Frech K, Karas H, Wingender E & Werner T (1995) Matind and Matinspector: New Fast and Versatile Tools for Detection of Consensus Matches in Nucleotide Sequence Data. Nucleic Acids Res 23:4878-4884.
Pritchard J B & Renfro J L (1983) Renal Sulfate Transport at the Basolateral Membrane Is Mediated by Anion Exchange. Proc Natl Acad Sci USA 80:2603-2607.
Pritchard J B & Miller D S (1993) Mechanisms Mediating Renal Secretion of Organic Anions and Cations. Physiol Rev 73:765-796.
Remington J P, Osol A, Anderson J T, Hoover J E & Skolaut M W (1975) Remington's Pharmaceutical Sciences, 15th ed. Mack Pub. Co., Easton, Pa., United States of America.
Roberts L (1991) GRAIL Seeks out Genes Buried in DNA Sequence. Science 254:805.
Romero M F, Fong P, Berger U V, Hediger M A & Boron W F (1998) Cloning and Functional Expression of RNBC, an Electrogenic Na(⁺)—HCO₃⁻ Cotransporter from Rat Kidney. Am J Physiol 274:F425-432.
Romero M F, Hediger M A, Boulpaep E L & Boron W F (1997) Expression Cloning and Characterization of a Renal Electrogenic Na⁺/HCO₃⁻ Cotransporter. Nature 387:409-413.
Romero M F, Henry D, Nelson S, Harte P J, Dillon A K & Sciortino C M (2000) Cloning and Characterization of a Na⁺-Driven Anion Exchanger (NDAE1). A New Bicarbonate Transporter. J Biol Chem 275:24552-24559.
Rossolini G M, Cresti S, Ingianni A, Cattani P, Riccio M L & Satta G (1994) Use of Deoxyinosine-Containing Primers Vs Degenerate Primers for Polymerase Chain Reaction Based on Ambiguous Sequence Information. Mol Cell Probes 8:91-98.
Royaux I E, Wall S M, Karniski L P, Everett L A, Suzuki K, Knepper M A & Green E D (2001) Pendrin, Encoded by the Pendred Syndrome Gene, Resides in the Apical Region of Renal Intercalated Cells and Mediates Bicarbonate Secretion. Proc Natl Acad Sci USA 98:4221-4226.
Saier M H, Jr., Eng B H, Fard S, Garg J, Haggerty D A, Hutchinson W J, Jack D L, Lai E C, Liu H J, Nusinew D P, Omar A M, Pao S S, Paulsen I T, Quan J A, Sliwinski M, Tseng T T, Wachi S & Young G B (1999) Phylogenetic Characterization of Novel Transport Protein Families Revealed by Genome Analyses. Biochim Biophys Acta 1422:1-56.
Saltzman W M & Fung L K (1997) Polymeric Implants for Cancer Chemotherapy. Adv Drug Deliv Rev 26:209-230.
Sambrook et al. e (1989) Molecular Cloning: A Laboratory Manual. Cold Spring Harbor Laboratory Press, Cold Spring Harbor, N.Y., United States of America.
Sanchez-Olea R, Fuller C, Benos D & Pasantes-Morales H (1995) Volume-Associated Osmolyte Fluxes in Cell Lines with or without the Anion Exchanger. Am J Physiol 269:C1280-1286.
Saqi M A, Wild D L & Hartshorn M J (1999) Protein Analyst—a Distributed Object Environment for Protein Sequence and Structure Analysis. Bioinformatics 15:521-522.
Satoh H, Susaki M, Shukunami C, Iyama K, Negoro T & Hiraki Y (1998) Functional Analysis of Diastrophic Dysplasia Sulfate Transporter. Its Involvement in Growth Regulation of Chondrocytes Mediated by Sulfated Proteoglycans. J Biol Chem 273:12307-12315.
Schneider C H & Eberle A N (1993) Peptides, 1992: Proceedings of the Twenty-Second European Peptide Symposium, Sep. 13-19, 1992, Interlaken, Switzerland. Escom, Leiden.
Schröder E & Lübke K (1965) The Peptides. Academic Press, New York.
Schug J & Overton G (1997) TESS: Transcription Element Search Software on the WWW. Technical Report CBIL-TR-1997-1001-V0.0. Computational Biology and Informatics Laboratory, School of Medicine, University of Pennsylvania, United States of America.
Scott D A & Karniski L P (2000) Human Pendrin Expressed in Xenopus Laevis Oocytes Mediates Chloride/Formate Exchange. Am J Physiol Cell Physiol 278:C207-211.
Scott D A, Wang R, Kreman T M, Sheffield V C & Karniski L P (1999) The Pendred Syndrome Gene Encodes a Chloride-Iodide Transport Protein. Nat Genet 21:440-443.
Siebens A W & Boron W F (1989) Depolarization-Induced Alkalinization in Proximal Tubules. II. Effects of Lactate and Sits. Am J Physiol 256:F354-365.
Silhavy T J, Berman M L, Enquist L W & Cold Spring Harbor Laboratory. (1984) Experiments with Gene Fusions. Cold Spring Harbor Laboratory, Cold Spring Harbor, N.Y., United States of America.
Smith T F & Waterman M (1981) Comparison of Biosequences. Adv Appl Math 2:482-489.
Soleimani M, Greeley T, Petrovic S, Wang Z, Amial H, Kopp P & Burnham C E (2001) Pendrin: An Apical Cl⁻/OH⁻/HCO₃⁻ Exchanger in the Kidney Cortex. Am J Physiol Renal Physiol 280:F356-364.
Songyang Z, Fanning A S, Fu C, Xu J, Marfatia S M, Chishti A H, Crompton A, Chan A C, Anderson J M & Cantley L C (1997) Recognition of Unique Carboxyl-Terminal Motifs by Distinct PDZ Domains. Science 275:73-77.
Songyang Z, Shoelson S E, Chaudhuri M, Gish G, Pawson T, Haser W G, King F, Roberts T, Ratnofsky S, Lechleider R J & et al. (1993) SH2 Domains Recognize Specific Phosphopeptide Sequences. Cell 72:767-778.
Speight T M, Holford N H G & Avery G S (1997) Avery's Drug Treatment: A Guide to the Properties, Choice. Therapeutic Use and Economic Value of Drugs in Disease Management, 4th/ed. Adis International, Auckland; Philadelphia, Pa., United States of America.
Stoneking M, Hedgecock D, Higuchi R G, Vigilant L & Erlich H A (1991) Population Variation of Human Mtdna Control Region Sequences Detected by Enzymatic Amplification and Sequence-Specific Oligonucleotide Probes. Am J Hum Genet 48:370-382.
Strange K (1998) Molecular Identity of the Outwardly Rectifying, Swelling-Activated Anion Channel: Time to Reevaluate pICIn. J Gen Physiol 111:617-622.
Tallgren L G (1980) Inorganic Sulphates in Relation to the Serum Thyroxine Level and in Renal Failure. Acta Med Scand Suppl 640:1-100.
Tam P, Monck M, Lee D, Ludkovski 0, Leng E C, Clow K, Stark H, Scherrer P, Graham R W & Cullis P R (2000) Stabilized Plasmid-Lipid Particles for Systemic Gene Therapy. Gene Ther 7:1867-1874.
Tarran R, Grubb B R, Gatzy J T, Davis C W & Boucher R C (2001) The Relative Roles of Passive Surface Forces and Active Ion Transport in the Modulation of Airway Surface Liquid Volume and Composition. J Gen Physiol 118:223-236.
Tijssen (1993) Laboratory Techniques in Biochemistry and Molecular Biology-Hybridization with Nucleic Acid Probes. Elsevier, N.Y.
Tsuganezawa H, Kobayashi K, Iyori M, Araki T, Koizumi A, Watanabe S, Kaneko A, Fukao T, Monkawa T, Yoshida T, Kim D K, Kanai Y, Endou H, Hayashi M & Saruta T (2001) A New Member of the HCO₃(⁻) Transporter Superfamily Is an Apical Anion Exchanger of Beta-Intercalated Cells in the Kidney. J Biol Chem 276:8180-8189.
Uberbacher E C, Xu Y & Mural R J (1996) Discovering and Understanding Genes in Human DNA Sequence Using GRAIL. Methods Enzymol 266:259-281.
Ulrich C D, 2nd (2000) Bicarbonate Secretion and CFTR: Continuing the Paradigm Shift. Gastroenterology 118:1258-1261.
U.S. Pat. No. 4,196,265
U.S. Pat. No. 4,235,871
U.S. Pat. No. 4,455,842
U.S. Pat. No. 4,551,482
U.S. Pat. No. 4,554,101
U.S. Pat. No. 4,736,866
U.S. Pat. No. 4,946,778
U.S. Pat. No. 5,011,634
U.S. Pat. No. 5,091,513
U.S. Pat. No. 5,111,867
U.S. Pat. No. 5,132,405
U.S. Pat. No. 5,162,215
U.S. Pat. No. 5,223,409
U.S. Pat. No. 5,260,203
U.S. Pat. No. 5,264,563
U.S. Pat. No. 5,489,742
U.S. Pat. No. 5,490,840
U.S. Pat. No. 5,498,538
U.S. Pat. No. 5,510,103
U.S. Pat. No. 5,550,316
U.S. Pat. No. 5,573,933
U.S. Pat. No. 5,574,172
U.S. Pat. No. 5,629,145
U.S. Pat. No. 5,632,991
U.S. Pat. No. 5,614,396
U.S. Pat. No. 5,625,125
U.S. Pat. No. 5,648,061
U.S. Pat. No. 5,650,489
U.S. Pat. No. 5,651,991
U.S. Pat. No. 5,667,988
U.S. Pat. No. 5,677,427
U.S. Pat. No. 5,688,931
U.S. Pat. No. 5,702,892
U.S. Pat. No. 5,714,166
U.S. Pat. No. 5,741,957
U.S. Pat. No. 5,738,996
U.S. Pat. No. 5,747,334
U.S. Pat. No. 5,756,291
U.S. Pat. No. 5,776,859
U.S. Pat. No. 5,780,225
U.S. Pat. No. 5,780,242
U.S. Pat. No. 5,786,387
U.S. Pat. No. 5,824,483
U.S. Pat. No. 5,834,228
U.S. Pat. No. 5,840,479
U.S. Pat. No. 5,849,877
U.S. Pat. No. 5,855,900
U.S. Pat. No. 5,858,410
U.S. Pat. No. 5,858,670
U.S. Pat. No. 5,872,011
U.S. Pat. No. 5,892,019
U.S. Pat. No. 5,922,254
U.S. Pat. No. 5,922,356
U.S. Pat. No. 5,922,545
U.S. Pat. No. 5,948,635
U.S. Pat. No. 5,948,647
U.S. Pat. No. 5,948,767
U.S. Pat. No. 5,985,279
U.S. Pat. No. 5,994,392
U.S. Pat. No. 6,054,561
U.S. Pat. No. 6,056,938
U.S. Pat. No. 6,061,581
U.S. Pat. No. 6,057,098
U.S. Pat. No. 6,071,890
U.S. Pat. No. 6,090,925
U.S. Pat. No. 6,106,866
U.S. Pat. No. 6,107,059
U.S. Pat. No. 6,120,787
U.S. Pat. No. 6,127,339
U.S. Pat. No. 6,132,766
U.S. Pat. No. 6,140,123
U.S. Pat. No. 6,156,511
U.S. Pat. No. 6,168,912
U.S. Pat. No. 6,174,708
U.S. Pat. No. 6,176,089
U.S. Pat. No. 6,180,084
U.S. Pat. No. 6,180,348
U.S. Pat. No. 6,190,700
U.S. Pat. No. 6,197,333
U.S. Pat. No. 6,200,598
U.S. Pat. No. 6,210,707
U.S. Pat. No. 6,214,553
U.S. Pat. No. 6,217,886
U.S. Pat. No. 6,221,958
U.S. Pat. No. 6,238,704
U.S. Pat. No. 6,238,705
U.S. Pat. No. 6,245,740
U.S. Pat. No. 6,262,127
U.S. Pat. No. 6,267,981
U.S. Pat. No. 6,287,587
U.S. Pat. No. 6,296,832
U.S. Pat. No. 6,296,842
U.S. Pat. No. 6,312,713
U.S. Pat. No. 6,335,035
Verkman A S (1990) Development and Biological Applications of Chloride-Sensitive Fluorescent Indicators. Am J Physiol 259:C375-388.
Walker M R & Rapley R (1993) Molecular and Antibody Probes in Diagnosis. Wiley, Chichester/New York.
Wang D G, Fan J B, Siao C J, Berno A, Young P; Sapolsky R, Ghandour G, Perkins N, Winchester E, Spencer J, Kruglyak L, Stein L, Hsie L, Topaloglou T, Hubbell E, Robinson E, Mittmann M, Morris M S, Shen N, Kilburn D, Rioux J, Nusbaum C, Rozen S, Hudson T J, Lander E S & et al. (1998) Large-Scale Identification, Mapping, and Genotyping of Single-Nucleotide Polymorphisms in the Human Genome. Science 280:1077-1082.
Wang S, Yue H, Derin R B, Guggino W B & Li M (2000) Accessory Protein Facilitated CFTR-CFTR Interaction, a Molecular Mechanism to Potentiate the Chloride Channel Activity. Cell 103:169-179.
Wang K & Wondergem R (1993) Redistribution of Hepatocyte Chloride During L-Alanine Uptake. J Membr Biol 135:237-244.
Weiss M S, Wacker T, Weckesser J, Welte W & Schulz G E (1990) The Three-Dimensional Structure of Porin from Rhodobacter Capsulatus at 3 a Resolution. FEBS Lett 267:268-272.
Worrall T A, Cotter R J & Woods A S (1998) Purification of Contaminated Peptides and Proteins on Synthetic Membrane Surfaces for Matrix-Assisted Laser Desorption/Ionization Mass Spectrometry. Anal Chem 70:750-756.
Wyckoff H W, Hirs C H W & Timasheff S N (1985) Diffraction Methods for Biological Macromolecules. Academic Press, Orlando, Fla., United States of America.
Xu Y, Mural R, Shah M & Uberbacher E (1994) Recognizing Exons in Genomic Sequence Using GRAIL II. Genet Eng 16:241-253.
Yaffe M B, Rittinger K, Volinia S, Caron P R, Aitken A, Leffers H, Gamblin S J, Smerdon S J & Cantley L C (1997) The Structural Basis for 14-3-3:Phosphopeptide Binding Specificity. Cell 91:961-971.
Yuan B, Thomas J P, von Kodolitsch Y & Pyeritz R E (1999) Comparison of Heteroduplex Analysis, Direct Sequencing, and Enzyme Mismatch Cleavage for Detecting Mutations in a Large Gene, FBN1. Hum Mutat 14:440-446.
Zheng J, Shen W, He D Z, Long K B, Madison L D & Dallos P (2000) Prestin Is the Motor Protein of Cochlear Outer Hair Cells. Nature 405:149-155.

It will be understood that various details of the invention can be changed without departing from the scope of the invention. Furthermore, the foregoing description is for the purpose of illustration only, and not for the purpose of limitation—the invention being defined by the claims appended hereto.

1. An isolated SLC26A7 polypeptide.

2. The isolated SLC26A7 polypeptide of claim 1, further comprising:
(a) a polypeptide of SEQ ID NO:2 or 4;
(b) a polypeptide substantially identical to SEQ ID NO:2 or 4;
(c) a polypeptide encoded by a nucleic acid molecule of SEQ ID NO:1 or 3; or
(d) a polypeptide encoded by a nucleic acid molecule substantially identical to SEQ ID NO:1 or 3.

3. The isolated SLC26A7 polypeptide of claim 1, wherein the SLC26A7 polypeptide is encoded by an isolated nucleic acid molecule selected from the group consisting of:
(a) an isolated nucleic acid molecule encoding a polypeptide of SEQ ID NO:2 or 4;
(b) an isolated nucleic acid molecule of SEQ ID NO:1 or 3;
(c) an isolated nucleic acid molecule which hybridizes to a nucleic acid sequence of SEQ ID NO:1 or 3 under wash stringency conditions represented by a wash solution having less than about 200 mM salt concentration and a wash temperature of greater than about 45° C., and which encodes a SLC26A7 polypeptide; and
(d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A7 polypeptide encoded by the isolated nucleic acid of one of (a), (b), and (c) above.

4. The isolated SLC26A7 polypeptide of claim 1, further comprising a functional SLC26A7 polypeptide.

5. The functional SLC26A7 polypeptide of claim 4, wherein the functional property comprises chloride transport.

6. A system for recombinant expression of a SLC26A7 polypeptide, the system comprising:
(a) a SLC26A7 polypeptide; and
(b) a host cell comprising the SLC26A7 polypeptide.

7. An isolated SLC26A7nucleic acid molecule.

8. The isolated SLC26A7 nucleic acid molecule of claim 7, further comprising a nucleic acid molecule encoding a SLC26A7 polypeptide.

9. The isolated SLC26A7 nucleic acid of claim 7, further comprising:
(a) a nucleotide sequence of SEQ ID NO:1 or 3; or
(b) a nucleotide sequence substantially identical to SEQ ID NO:1 or 3.

10. The isolated SLC26A7 nucleic acid molecule of claim 7, further comprising a nucleic acid molecule selected from the group consisting of:
(a) an isolated nucleic acid molecule encoding a polypeptide of SEQ ID NO:2 or 4;
(b) an isolated nucleic acid molecule of SEQ ID NO:1 or 3;
(c) an isolated nucleic acid molecule which hybridizes to a nucleic acid sequence of SEQ ID NO:1 or 3 under wash stringency conditions represented by a wash solution having less than about 200 mM salt concentration and a wash temperature of greater than about 45° C., and which encodes a SLC26A7 polypeptide; and
(d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A7 polypeptide encoded by the isolated nucleic acid of one of (a), (b), and (c) above.

11. A method for detecting a SLC26A7 nucleic acid molecule, the method comprising:
(a) procuring a biological sample having nucleic acid material;
(b) hybridizing the nucleic acid molecule of SEQ ID NO:1 or 3 under stringent hybridization conditions to the biological sample of (a), thereby forming a duplex structure between the nucleic acid of SEQ ID NO:1 or 3 and a nucleic acid within the biological sample; and
(c) detecting the duplex structure of (b), whereby a SLC26A7 nucleic acid molecule is detected.

12. A method for producing an antibody that specifically recognizes a SLC26A7 polypeptide, the method comprising:
(a) recombinantly or synthetically producing a SLC26A7 polypeptide;
(b) formulating the polypeptide of (a) whereby it is an effective immunogen;
(c) administering to an animal the formulation of (b) to generate an immune response in the animal comprising production of antibodies, wherein antibodies are present in the blood serum of the animal; and
(d) collecting the blood serum from the animal of (c) comprising antibodies that specifically recognize a SLC26A7 polypeptide.

13. The method of claim 12, wherein the SLC26A7 polypeptide comprises:
(a) a polypeptide encoded by a nucleic acid of SEQ ID NO:1 or 3;
(b) a polypeptide encoded by a nucleic acid substantially identical to SEQ ID NO:1 or 3;
(c) a polypeptide comprising an amino acid sequence of SEQ ID NO:2 or 4; or
(d) a polypeptide substantially identical to SEQ ID NO:2 or 4.

14. The method of claim 12, wherein the SLC26A7 polypeptide is encoded by an isolated nucleic acid segment selected from the group consisting of:
(a) an isolated nucleic acid molecule encoding a polypeptide of SEQ ID NO:2 or 4;
(b) an isolated nucleic acid molecule of SEQ ID NO:1 or 3;
(c) an isolated nucleic acid molecule which hybridizes to a nucleic acid sequence of SEQ ID NO:1 or 3 under wash stringency conditions represented by a wash solution having less than about 200 mM salt concentration and a wash temperature of greater than about 45° C., and which encodes a functional SLC26A7 polypeptide; and
(d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A7 polypeptide encoded by the isolated nucleic acid of one of (a), (b), and (c) above.

15. An antibody produced by the method of claim 12.

16. A method for detecting a level of a SLC26A7 polypeptide, the method comprising
(a) obtaining a biological sample having peptidic material;
(b) detecting a SLC26A7 polypeptide in the biological sample of (a) by immunochemical reaction with the antibody of claim 13, whereby an amount of SLC26A7 polypeptide in a sample is determined.

17. A method for identifying a modulator of a SLC26A7 polypeptide, the method comprising:
(a) providing a recombinant expression system whereby a SLC26A7 polypeptide is expressed in a host cell;
(b) providing a test substance to the system of (a);
(c) assaying a level or quality of SLC26A7 function in the presence of the test substance;
(d) comparing the level or quality of SLC26A7 function in the presence of the test substance with a control level or quality of SLC26A7 function; and
(e) identifying a test substance as an anion transport modulator by determining a level or quality of SLC26A7 function in the presence of the test substance as significantly changed when compared to a control level or quality of SLC26A7 function.

18. The method of claim 17, wherein the SLC26A7 polypeptide comprises:
(a) a polypeptide encoded by a nucleic acid of SEQ ID NO:1 or 3;
(b) a polypeptide encoded by a nucleic acid substantially identical to SEQ ID NO:1 or 3;
(c) a polypeptide comprising an amino acid sequence of SEQ ID NO:2 or 4; or
(d) a polypeptide substantially identical to SEQ ID NO:2 or 4.

19. The method of claim 17, wherein the SLC26A7 polypeptide is encoded by an isolated nucleic acid segment selected from the group consisting of:
(a) an isolated nucleic acid molecule encoding a polypeptide of SEQ ID NO:2 or 4;
(b) an isolated nucleic acid molecule of SEQ ID NO:1 or 3;
(c) an isolated nucleic acid molecule which hybridizes to a nucleic acid sequence of SEQ ID NO:1 or 3 under wash stringency conditions represented by a wash solution having less than about 200 mM salt concentration and a wash temperature of greater than about 45° C., and which encodes a functional SLC26A7 polypeptide; and
(d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A7 polypeptide encoded by the isolated nucleic acid of one of (a), (b), and (c) above.

20. The method of claim 17, wherein the host cell comprises a mammalian cell.

21. The method of claim 20, wherein the mammalian cell comprises a human cell.

22. An anion transporter modulator identified by the method of claim 17.

23. A method for modulating anion transport activity in a subject, the method comprising:
(a) preparing a composition comprising a modulator identified according to the method of claim 17, and a pharmaceutically acceptable carrier;
(b) administering an effective dose of the composition to a subject, whereby anion transport activity is altered in the subject.

24. The method of claim 23, wherein the subject is a mammal.

25. The method of claim 24, wherein the subject is a human.

26. A method for identifying an anion exchanger modulator, the method comprising:
(a) exposing a SLC26A7 polypeptide to one or more test substances;
(b) assaying binding of a test substance to the isolated SLC26A7 polypeptide; and
(c) selecting a candidate substance that demonstrates specific binding to the SLC26A7 polypeptide.

27. The method of claim 26, wherein the SLC26A7 polypeptide comprises:
(a) a polypeptide encoded by a nucleic acid of SEQ ID NO:1 or 3;
(b) a polypeptide encoded by a nucleic acid substantially identical to SEQ ID NO:1 or 3;
(c) a polypeptide comprising an amino acid sequence of SEQ ID NO:2 or 4; or
(d) a polypeptide substantially identical to SEQ ID NO:2 or 4.

28. The method of claim 26, wherein the SLC26A7 polypeptide is encoded by an isolated nucleic acid segment selected from the group consisting of:
(a) an isolated nucleic acid molecule encoding a polypeptide of SEQ ID NO:2 or 4;
(b) an isolated nucleic acid molecule of SEQ ID NO:1 or 3;
(c) an isolated nucleic acid molecule which hybridizes to a nucleic acid sequence of SEQ ID NO:1 or 3 under wash stringency conditions represented by a wash solution having less than about 200 mM salt concentration and a wash temperature of greater than about 45° C., and which encodes a functional SLC26A7 polypeptide; and
(d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A7 polypeptide encoded by the isolated nucleic acid of (a), (b), and (c) above.

29. The method of claim 26, wherein the host cell comprises a mammalian cell.

30. The method of claim 29, wherein the mammalian cell comprises a human cell.

31. An anion transporter modulator identified by the method of claim 26.

32. A method for modulating anion transport activity in a subject, the method comprising:
(a) preparing a composition comprising a modulator identified according to the method of claim 26, and a pharmaceutically acceptable carrier;
(b) administering an effective dose of the composition to a subject, whereby anion transport activity is altered in the subject.

33. The method of claim 32, wherein the subject is a mammal.

34. The method of claim 33, wherein the subject is a human.

35. An isolated SLC26A9 polypeptide.

36. The isolated SLC26A9 polypeptide of claim 35, further comprising:
(a) a polypeptide of SEQ ID NO:6, 8, or 10;
(b) a polypeptide substantially identical to SEQ ID NO:6, 8, or 10;
(c) a polypeptide encoded by a nucleic acid molecule of SEQ ID NO:5, 7, or 9; or
(d) a polypeptide encoded by a nucleic acid molecule substantially identical to SEQ ID NO:5, 7, or 9.

37. The isolated SLC26A9 polypeptide of claim 35, wherein the SLC26A9 polypeptide is encoded by an isolated nucleic acid molecule selected from the group consisting of:
(a) an isolated nucleic acid molecule encoding a polypeptide of SEQ ID NO:6, 8, or 10;
(b) an isolated nucleic acid molecule of SEQ ID NO:5, 7, or 9;
(c) an isolated nucleic acid molecule which hybridizes to a nucleic acid sequence of SEQ ID NO:5, 7, or 9 under wash stringency conditions represented by a wash solution having less than about 200 mM salt concentration and a wash temperature of greater than about 45° C., and which encodes a SLC26A9 polypeptide; and
(d) an isolated nucleic acid molecule differing by at least one functionally equivalent codon from the isolated nucleic acid molecule of one of (a), (b), or (c) above in nucleic acid sequence due to the degeneracy of the genetic code, and which encodes a SLC26A9 polypeptide encoded by the isolated nucleic acid of one of (a), (b), or (c) above.

38. The isolated SLC26A9 polypeptide of claim 37, further comprising a functional SLC26A9 polypeptide.

39. The functional SLC26A9 polypeptide of claim 38, wherein the functional property comprises chloride transport.

40. The functional SLC26A9 polypeptide of claim 38, wherein the functional property comprises bicarbonate ion transport.

41. A system for recombinant expression of a SLC26A9 polypeptide, the system comprising:
(a) a SLC26A9 polypeptide; and
(b) a host cell comprising the SLC26A9 polypeptide.

42. An isolated SLC26A9 nucleic acid molecule.

43. The isolated SLC26A9 nucleic acid molecule of claim 42, further comprising a nucleic acid molecule encoding a SLC26A9 polypeptide.

44. The isolated SLC26A9 nucleic acid of claim 42, further comprising:
(a) a nucleotide sequence of SEQ ID NO:5, 7, or 9; or
(b) a nucleotide sequence substantially identical to SEQ ID NO:5, 7, or 9.

45. The isolated SLC26A9 nucleic acid molecule of claim 42, further comprising a nucleic acid molecule selected from the group consisting of:
(a) an isolated nucleic acid molecule encoding a polypeptide of SEQ ID NO:6,8, or 10;
(b) an isolated nucleic acid molecule of SEQ ID NO:5, 7, or 9;
(c) an isolated nucleic acid molecule which hybridizes to a nucleic acid sequence of SEQ ID NO:5, 7, or 9 under wash stringency conditions represented by a wash solution having less than about 200 mM salt concentration and a wash temperature of greater than about 45° C., and which encodes a SLC26A9 polypeptide; and
(d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A9 polypeptide encoded by the isolated nucleic acid of one of (a), (b), and (c) above.

46. A method for detecting a SLC26A9 nucleic acid molecule, the method comprising:
(a) procuring a biological sample having nucleic acid material;
(b) hybridizing the nucleic acid molecule of SEQ ID NO:5, 7, or 9 under stringent hybridization conditions to the biological sample of (a), thereby forming a duplex structure between the nucleic acid of SEQ ID NO:5, 7, or 9 and a nucleic acid within the biological sample; and
(c) detecting the duplex structure of (b), whereby a SLC26A9 nucleic acid molecule is detected.

47. A method for producing an antibody that specifically recognizes a SLC26A9 polypeptide, the method comprising:
(a) recombinantly or synthetically producing a SLC26A9 polypeptide;
(b) formulating the polypeptide of (a) whereby it is an effective immunogen;
(c) administering to an animal the formulation of (b) to generate an immune response in the animal comprising production of antibodies, wherein antibodies are present in the blood serum of the animal; and
(d) collecting the blood serum from the animal of (c) comprising antibodies that specifically recognize a SLC26A9 polypeptide.

48. The method of claim 47, wherein the SLC26A9 polypeptide comprises:
(a) a polypeptide encoded by a nucleic acid of SEQ ID NO:5, 7, or 9;
(b) a polypeptide encoded by a nucleic acid substantially identical to SEQ ID NO:5, 7, or 9;
(c) a polypeptide comprising an amino acid sequence of SEQ ID NO:6, 8, or 10; or
(d) a polypeptide substantially identical to SEQ ID NO:6, 8, or 10.

49. The method of claim 47, wherein the SLC26A9 polypeptide is encoded by an isolated nucleic acid segment selected from the group consisting of:
(a) an isolated nucleic acid molecule encoding a polypeptide of SEQ ID NO:6, 8, or 10;
(b) an isolated nucleic acid molecule of SEQ ID NO:5, 7, or 9;
(c) an isolated nucleic acid molecule which hybridizes to a nucleic acid sequence of SEQ ID NO:5, 7, or 9 under wash stringency conditions represented by a wash solution having less than about 200 mM salt concentration and a wash temperature of greater than about 45° C., and which encodes a functional SLC26A9 polypeptide; and
(d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A9 polypeptide encoded by the isolated nucleic acid of one of (a), (b), and (c) above.

50. An antibody produced by the method of claim 47.

51. A method for detecting a level of a SLC26A9 polypeptide, the method comprising
(a) obtaining a biological sample having peptidic material;
(b) detecting a SLC26A9 polypeptide in the biological sample of (a) by immunochemical reaction with the antibody of claim 50, whereby an amount of SLC26A9 polypeptide in a sample is determined.

52. A method for identifying a modulator of a SLC26A9 polypeptide, the method comprising:
(a) providing a recombinant expression system whereby a SLC26A9 polypeptide is expressed in a host cell;
(b) providing a test substance to the system of (a);
(c) assaying a level or quality of SLC26A9 function in the presence of the test substance;
(d) comparing the level or quality of SLC26A9 function in the presence of the test substance with a control level or quality of SLC26A9 function; and
(e) identifying a test substance as an anion transport modulator by determining a level or quality of SLC26A9 function in the presence of the test substance as significantly changed when compared to a control level or quality of SLC26A9 function.

53. The method of claim 52, wherein the SLC26A9 polypeptide comprises:
(a) a polypeptide encoded by a nucleic acid of SEQ ID NO:5, 7, or 9;
(b) a polypeptide encoded by a nucleic acid substantially identical to SEQ ID NO:5, 7, or 9;
(c) a polypeptide comprising an amino acid sequence of SEQ ID NO:6, 8, or 10; or
(d) a polypeptide substantially identical to SEQ ID NO:6, 8, or 10.

54. The method of claim 52, wherein the SLC26A9 polypeptide is encoded by an isolated nucleic acid segment selected from the group consisting of:
(a) an isolated nucleic acid molecule encoding a polypeptide of SEQ ID NO:6, 8, or 10;
(b) an isolated nucleic acid molecule of SEQ ID NO:5, 7, or 9;
(c) an isolated nucleic acid molecule which hybridizes to a nucleic acid sequence of SEQ ID NO:5, 7, or 9 under wash stringency conditions represented by a wash solution having less than about 200 mM salt concentration and a wash temperature of greater than about 45° C., and which encodes a functional SLC26A9 polypeptide; and
(d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A9 polypeptide encoded by the isolated nucleic acid of one of (a), (b), and (c) above.

55. The method of claim 52, wherein the host cell comprises a mammalian cell.

56. The method of claim 55, wherein the mammalian cell comprises a human cell.

57. An anion transporter modulator identified by the method of claim 52.

58. A method for modulating anion transport activity in a subject, the method comprising:
(a) preparing a composition comprising a modulator identified according to the method of claim 52, and a pharmaceutically acceptable carrier;
(b) administering an effective dose of the composition to a subject, whereby anion transport activity is altered in the subject.

59. The method of claim 58, wherein the subject is a mammal.

60. The method of claim 59, wherein the subject is a human.

61. A method for identifying an anion exchanger modulator, the method comprising:
(a) exposing a SLC26A9 polypeptide to one or more test substances;
(b) assaying binding of a test substance to the isolated SLC26A9 polypeptide; and
(c) selecting a candidate substance that demonstrates specific binding to the SLC26A9 polypeptide.

62. The method of claim 61, wherein the SLC26A9 polypeptide comprises:
(a) a polypeptide encoded by a nucleic acid of SEQ ID NO:5, 7, or 9;
(b) a polypeptide encoded by a nucleic acid substantially identical to SEQ ID NO:5, 7, or 9;
(c) a polypeptide comprising an amino acid sequence of SEQ ID NO:6, 8, or 10; or
(d) a polypeptide substantially identical to SEQ ID NO:6, 8, or 10.

63. The method of claim 61, wherein the SLC26A9 polypeptide is encoded by an isolated nucleic acid segment selected from the group consisting of:
(a) an isolated nucleic acid molecule encoding a polypeptide of SEQ ID NO:6, 8, or 10;
(b) an isolated nucleic acid molecule of SEQ ID NO:5, 7, or 9;
(c) an isolated nucleic acid molecule which hybridizes to a nucleic acid sequence of SEQ ID NO:5, 7, or 9 under wash stringency conditions represented by a wash solution having less than about 200 mM salt concentration and a wash temperature of greater than about 45° C., and which encodes a functional SLC26A9 polypeptide; and
(d) an isolated nucleic acid molecule differing by at least one functionally equivalent codon from the isolated nucleic acid molecule of one of (a), (b), and (c) above in nucleic acid sequence due to the degeneracy of the genetic code, and which encodes a SLC26A9 polypeptide encoded by the isolated nucleic acid of one of (a), (b), and (c) above.

64. The method of claim 61, wherein the host cell comprises a mammalian cell.

65. The method of claim 64, wherein the mammalian cell comprises a human cell.

66. An anion transporter modulator identified by the method of claim 61.

67. A method for modulating anion transport activity in a subject, the method comprising:
(a) preparing a composition comprising a modulator identified according to the method of claim 56, and a pharmaceutically acceptable carrier;
(b) administering an effective dose of the composition to a subject, whereby anion transport activity is altered in the subject.

68. The method of claim 67, wherein the subject is a mammal.

69. The method of claim 68, wherein the subject is a human.
